(19) United States
(12) Patent Application Publication
(10) Pub. No.: US 2004/0253578 A1
(43) Pub. Date: Dec. 16, 2004

(54) Dynamic action reference tools

(76) Inventors: Radclyffe L. Roberts, Seattle, WA (US); Paul De Figuereido, Kenmore, WA (US)

Correspondence Address: JONES DAY, 222 EAST 41ST ST, NEW YORK, NY, 10017, US

(21) Appl. No.: 10/474,298

(22) PCT Filed: Apr. 2, 2002
(86) PCT No.: PCT/US02/10566

Related U.S. Application Data
(60) Provisional application No. 60/281,133, filed on Apr. 2, 2001.
(60) Provisional application No. 60/281,342, filed on Apr. 3, 2001.

(51) Int. Cl. C12Q 1/70; C07H 21/04; C12N 9/00; C12N 7/00; C12N 15/74

(52) U.S. Cl. 435/5; 435/69.3; 435/183; 435/235.1; 435/320.1; 435/252.3; 536/23.72

(57) ABSTRACT

The present invention provides Dynamic Action Reference Tools, or DARTs, and methods of making and using DARTS. DARTs can be used, for example, for the isolation and analysis of nucleic acids, polypeptides, and the like, for regulating biological activities and investigating inter-molecular interactions, and the like. A DART is a molecule that includes a Molecular Shaft covalently linked to a Linkage Polypeptide that is covalently linked to a Molecular Point. DARTs, and DART libraries, can be formed and manipulated in vivo or in vitro. DARTs can be purified, and portions of DARTs can be exchanged with portions of other DARTs.

### CROSS-REFERENCES TO RELATED APPLICATIONS

This application claims the benefit of U.S. provisional application Nos. 60/281,133, filed Apr. 2, 2001, and 60/281,342, filed Apr. 3, 2001, under 35 U.S.C. § 119(e), which applications are incorporated by reference herein in their entirety.

### BACKGROUND OF THE INVENTION

The process of identifying new genes and determining the function of the genes, or gene products, can be modeled as an informational paradigm. Information about the gene (e.g., sequence information) is determined and used to acquire knowledge about the function of that gene. Determining sequence information about a gene provides little knowledge about the function of the gene product, however. Usually the function of a gene product is determined by separately characterizing the gene product. Such characterizations typically include comparing the gene sequence with the sequence of known genes, comparing the deduced gene product with known gene products, and/or genetic or biochemical analysis of the gene product. Further characterizations are usually required to identify the binding partner(s) and small molecule effectors associated with the gene product.

Another approach to characterizing genes and gene products is to use expression libraries. Such libraries can include lambda expression libraries, phage display libraries, and the like. A disadvantage of expression libraries, however, is that they require a biological intermediate to link informational components (i.e., nucleic acids) with the biological components responsible for molecular recognition and catalysis (i.e., proteins), and as such, cannot be deployed in systems where the presence of the biological intermediary may not be desirable. Thus, there is a need for nucleic acid constructs and methods of making and using such constructs that overcome this shortcoming, yet preserve the informational relationship between a gene and the gene product associated with that gene. The present invention satisfies this need and more.

### BRIEF SUMMARY OF THE INVENTION

The present invention provides Dynamic Action Reference Tools, referred to hereinafter as DARTs, which have a wide variety of applications including in the pharmaceutical, biomedical and biotechnology industries. A DART comprises a Molecular Shaft covalently linked to a Linkage Polypeptide covalently linked to a Molecular Point.

In one aspect, the present invention provides a DART in which the Linkage Polypeptide is an autocatalytic Linkage Polypeptide. In certain embodiments, the autocatalytic Linkage Polypeptide is a replication protein of a single-stranded DNA bacterial virus or bacteriophage. In another embodiment the Linkage Polypeptide is encoded by a gene A of an icosahedral bacteriophage. In specific modes of the embodiment, the icosahedral bacteriophage is φX174, φX, φG4, φS13, φP2, φ186 or φPM2. In another embodiment, the Linkage Polypeptide is encoded by a gene II of a filamentous bacteriophage. In specific modes of the embodiment, the filamentous bacteriophage is fd, f1, M13, ZJ/2, Ec9, AE2 or *A. In another embodiment, the Linkage Polypeptide is a Xanthomonas virus replication protein. In a specific mode of the embodiment, the Xanthomonas virus is XF1. In yet another embodiment, the Linkage Polypeptide is a Pseudomonas virus replication protein. In specific modes of the embodiment, the Pseudomonas virus is PF1, PF2 or PF3. In yet another embodiment, the Linkage Polypeptide is a Vibrioparahemolyticus virus replication protein. In a specific mode of the embodiment, the Vibrioparahemolyticus virus is V6.

In other embodiments, the autocatalytic Linkage Polypeptide is a replication protein of a single-stranded DNA mycobacterial virus or bacteriophage, such as, for example, the mycobacterial virus is MVL51.

The present invention further provides a DART comprising an autocatalytic Linkage Polypeptide, wherein the autocatalytic Linkage Polypeptide is a nicking or relaxase enzyme of a bacterial plasmid. In one embodiment, the plasmid is a narrow host range plasmid. In a specific mode of the embodiment, the narrow host range plasmid is F plasmid. The Linkage Polypeptide can be TraI. In other modes of the embodiment, the narrow host range plasmid is R1, R100, 61B4-K98, P307 or pED208. In another embodiment, the present invention provides a DART in which the Linkage Polypeptide is a nicking or relaxase enzyme of a broad host range plasmid. In one mode of the embodiment, the broad host range plasmid is RP4. The Linkage Polypeptide can be TraI. In other modes of the embodiment, the broad host range plasmid is Rp1, RK2, R18, R68 or R751. In another embodiment, the present invention provides a DART in which the Linkage Polypeptide is a nicking or relaxase enzyme of a mobilizable plasmid. In a specific mode of the embodiment, the mobilizable plasmid is IncQ; the nicking or relaxase enzyme can be, for example, MobA. In other modes of the embodiment, the mobilizable plasmid is RSF1010, R300B, or R1162. In another embodiment, the present invention provides a DART in which the Linkage Polypeptide is a nicking or relaxase enzyme of a R388-related plasmid, and can be, for example, R388 or IncQ. In a specific mode of the embodiment, the nicking or relaxase enzyme is TrwC. In a specific mode of the embodiment, the nicking or relaxase enzyme is a VirD2 enzyme of an A. tumefaciens Ti plasmid. In a typical embodiment the nicking or relaxase enzyme can be a variant (e.g. N-terminal truncation fragment) of VirD2 enzyme of an A. tumefaciens Ti plasmid, the variant retaining covalent linkage activity and lacking the ability to oligomerize or self-associate.

The present invention further provides a DART comprising an autocatalytic Linkage Polypeptide, wherein the autocatalytic Linkage Polypeptide is a viral capping protein. In one embodiment, the Linkage Polypeptide is a P protein of a Hepadnavirus. In a specific mode of the embodiment, the Hepadnavirus is Hepatitis B virus. In another embodiment, the Linkage Polypeptide is a VpG protein of a Picornavirus. In a typical mode of the embodiment, the Picornavirus is poliovirus. In other modes of the embodiment, the Picornavirus is apthovirus, cardiovirus, hepatitis A, enterovirus, rhinovirus or coxsackievirus. In another embodiment, the Linkage Polypeptide is an adenovirus terminal protein.

The present invention further provides a DART comprising an autocatalytic Linkage Polypeptide, wherein the autocatalytic Linkage Polypeptide is a site-specific recombinase that has been modified to form a covalent link with a nucleic acid comprising the recognition site of the recombinase. In one embodiment, the site-specific recombinase is phage 8 integrase. In other embodiments, the site-specific recombinase is CRE recombinase of phage P1, mammalian RAG1 or RAG2, an intergron integrase or FLP recombinase of Saccharomyces cerevisiae.

The present invention further provides a DART comprising an autocatalytic Linkage Polypeptide, wherein the autocatalytic Linkage Polypeptide is a site-specific endonuclease that has been modified to form a covalent link with a nucleic acid comprising the recognition site of the endonuclease. In one embodiment, the modified endonuclease is EcoRI. In another embodiment, the modified endonuclease is HO endonuclease of Saccharomyces cerevisiae. In other embodiments, the site-specific endonuclease is, for example, HindIII, ClaI, BamHI, BglII, BglI, PstI, XhoI, or XbaI.

The present invention further provides a DART comprising an autocatalytic Linkage Polypeptide, wherein the autocatalytic Linkage Polypeptide is topoisomerase or integrase that has been modified to form a covalent link with a nucleic acid.

The present invention further provides a DART comprising an autocatalytic Linkage Polypeptide, wherein the autocatalytic Linkage Polypeptide is a geminivirus replication protein, a caulimovirus replication protein, a badnavirus replication protein, a reovirus replication protein, a phytovirus replication protein, a fijivirus replication protein, an oryzavirus replication protein, a partitivirus replication protein, an alphacryptovirus replication protein, a betacryptovirus replication protein, a rhabdovirus replication protein, a nucleorhabdovirus replication protein, a bunyavirus replication protein, a topsovirus replication protein, a tenuivirus replication protein, a sequivirus replication protein, a tombusvirus replication protein, a dianthovirus replication protein, an enamovirus replication protein, an idaeovirus replication protein, a luteovirus replication protein, a machlomovirus replication protein, a marafivirus replication protein, a necrovirus replication protein, a sobemovirus replication protein, a tymovirus replication protein, an umbravirus replication protein, a bromovirus replication protein, a comovirus replication protein, a tobamovirus replication protein, a hordeivirus replication protein, a tobravirus replication protein, a furoivirus replication protein, a potexvirus replication protein, a capillovirus replication protein, a trichovirus replication protein, a carlavirus replication protein, a potyvirus replication protein, a closterovirus replication protein, a parvovirus replication protein, a baculovirus replication protein, a nudivirus replication protein, a polydnavirus replication protein, a poxvirus replication protein, an ascovirus replication protein, an iridovirus replication protein, a birnavirus replication protein, a togavirus replication protein, a replication protein of a flavivirus family member, a picornavirus replication protein, a tetravirus replication protein, or a nodavirus replication protein.

In another embodiment, the Linkage Polypeptide is a reovirus replication protein. In one mode of the embodiment, the reovirus is a plant reovirus. In another mode of the embodiment, the reovirus is an insect reovirus. In another embodiment, the Linkage Polypeptide is a rhabdovirus replication protein. In one mode of the embodiment, the rhabdovirus is a plant rhabdovirus. In another mode of the embodiment, the rhabdovirus is an insect rhabdovirus. In another embodiment, the Linkage Polypeptide is a bunyavirus replication protein. In one mode of the embodiment, the bunyavirus is a plant bunyavirus. In another mode of the embodiment, the bunyavirus is an insect bunyavirus. In yet another embodiment, the Linkage Polypeptide is a replication protein of a flavivirus family member (i.e., of the Flaviviridae family). In one mode of the embodiment, the flavivirus family member is a flavivirus. In another mode of the embodiment, the flavivirus family member is a pestivirus. In certain embodiments, the present invention provides a DART in which the Linkage Polypeptide is not a protein which is naturally attached to the Molecule Shaft.

The present invention further provides a DART in which the Linkage Polypeptide is a fusion protein comprising a protein that catalyzes the formation of a covalent link between itself and a nucleic acid, and an accessory protein. In one embodiment, the fusion protein is virD2 and the accessory protein is virD1 that catalyzes the formation of a covalent link between itself and a nucleic acid.

The present invention further provides a DART in which the Linkage Polypeptide is an autocatalytic Linkage Polypeptide that selectively catalyzes the formation of a covalent bond between itself and a single stranded nucleic acid. In one embodiment, the nucleic acid is RNA. In another embodiment, the nucleic acid is DNA.

The present invention further provides a DART in which the Linkage Polypeptide is an autocatalytic Linkage Polypeptide that selectively catalyzes the formation of a covalent bond between itself and a double stranded nucleic acid. In one embodiment, the nucleic acid is RNA. In another embodiment, the nucleic acid is DNA. In yet another embodiment, the nucleic acid is DNA/RNA hybrid.

The present invention further provides a DART in which the Linkage Polypeptide is an autocatalytic Linkage Polypeptide that covalently links itself to the 3′-end of a nucleic acid.

The present invention further provides a DART in which the Linkage Polypeptide is an autocatalytic Linkage Polypeptide that covalently links itself to the 5′-end of a nucleic acid.

The present invention further provides a DART in which the Linkage Polypeptide is an autocatalytic Linkage Polypeptide that is a fusion protein. In one embodiment, the fusion protein comprises a DNA recognition domain, a catalytic cleavage domain and a joining domain. In one mode of the embodiment, the cleavage domain and the joining domain are the same. In another mode of the embodiment, the DNA recognition domain comprises a zinc finger motif. In yet another mode of the embodiment, the catalytic cleavage domain is a non-specific DNA cleavage domain, for example a non-specific DNA cleavage domain is derived from a class II restriction endonuclease, such as FokI, or a non-specific DNA cleavage domain derived from a class III restriction endonuclease. In yet another mode of the embodiment, the DNA recognition domain comprises a virD2-class DNA recognition motif, the catalytic cleavage domain comprises a virD2-class catalytic cleavage motif, the catalytic joining domain comprises a virD2-class catalytic joining motif, or a combination of the foregoing.

The present invention further provides a DART in which the Linkage Polypeptide is an autocatalytic Linkage Polypeptide that is a fusion protein comprising an affinity tag or a sequence that regulates the Linkage Polypeptide's subcellular localization, such as a nuclear localization signal or a secretory signal sequence, or a protease recognition site.

The present invention further provides a DART in which the Linkage Polypeptide is a non-autocatalytic Linkage Polypeptide. In one embodiment, the non-autocatalytic Linkage Polypeptide is a substrate for a transcatalytic linking protein. In one mode of the embodiment, the Linkage Polypeptide is a non-catalytic VirD2 mutant and the transcatalytic linking protein is a VirD2 mutant lacking an acceptor residue for covalent linkage to a nucleic acid. In another embodiment, the non-autocatalytic Linkage Polypeptide is a substrate for a trans-complementary linking protein. In one mode of the embodiment, the Linkage Polypeptide comprises a Hepadnavirus terminal protein domain and the trans-complementary linking protein comprises a Hepadnavirus reverse transcriptase domain, or more particularly Hepatitis B Virus terminal protein domain and Hepatitis B Virus reverse transcriptase domain.

The present invention provides a DART in which the Molecular Shaft is DNA, RNA or an RNA/DNA hybrid. The present invention further provides a DART in which the Molecular Shaft is single stranded, double stranded, or partially double stranded. The present invention further provides a DART in which the Molecular Shaft can be, for example, 10 to 10,000 bases or base pairs, 20 to 7,500 bases or base pairs, 50 to 5,000 bases or base pairs, 100 to 1,000 bases or base pairs, 250 to 500 bases or base pairs, 100 to 250 bases or base pairs, 50 to 100 bases or base pairs, 20 to 50 bases or base pairs, 10 to 20 bases or base pairs in length. In yet other embodiments, the present invention provides a DART in which the Molecular Shaft is, for example, 500 to 1,000 bases or base pairs, 1,000 to 5,000 bases or base pairs, 5,000 to 7,500 bases or base pairs, or 7,500 to 10,000 bases or base pairs in length.

The present invention further provides a DART in which the Molecular Shaft encodes at least a portion of the Molecular Point. The Molecular Shaft can encode a fusion polypeptide comprising the Linkage Polypeptide and the Molecular Point. The present invention further provides a DART in which the Molecular Shaft comprises a detectably-labeled nucleotide. In one embodiment, the detectably-labeled nucleotide comprises a fluorescent moiety, such as Cy3, Cy5, rhodamine or fluorescein.

The present invention further provides a DART in which the Molecular Shaft further comprises a Recognition Sequence Motif, a primer annealing site, a restriction endonuclease recognition site, a nucleic acid sequence encoding an epitope tag, a nucleic acid sequence encoding a linker polypeptide, a nucleic acid encoding a protease recognition site, or a nucleic acid sequence encoding a nuclear localization signal.

The present invention further provides a DART in which the Molecular Point comprises a polypeptide. In one embodiment, the Molecular Point comprises an antibody. The present invention further provides a DART in which the Molecular Point comprises a polypeptide of unknown function or a mutant or variant of a known protein. In yet other embodiments, the present invention provides a DART in which the Molecular Point comprises Green Fluorescent Protein (GFP) or RNase.

The present invention further provides a DART which is a polyvalent DART, an RNase DART, an RNase H antisense DART, or a self-referential DART.

In certain embodiments, the present invention provides a DART that is immobilized to a solid surface. In another embodiment, the present invention provides a DART in solution. Where a DART is immobilized on a solid surface, in various embodiments, the solid surface can be, for example, an agarose bead, a polystyrene bead, a magnetic bead, a glass slide, a glass bead, a silicon wafer, a microtiter plate, a nitrocellulose membrane, a nylon membrane, or a PVDF membrane.

The present invention further provides an expression construct comprising a preMS and encoding a self-referential DART, the DART comprising an open reading frame encoding the Linkage Polypeptide and the Molecular Point, the open reading frame being operatively linked to a promoter. In one embodiment, the expression construct is in the form of a vector comprising a selection marker and an origin of replication. The selection marker can be, for example, a positive selection marker and/or a negative selection marker. In another mode of the embodiment, the origin of replication is suitable for replication in a eukaryotic cell and/or in a prokaryotic cell.

The present invention further provides a host cell comprising a first nucleic acid and a second nucleic acid, the first nucleic acid comprising an open reading frame encoding a fusion protein, the fusion protein comprising a Linkage Polypeptide covalently linked to a Molecular Point, and the second nucleic acid comprising a sequence encoding a preMS. In one embodiment, the first nucleic acid and the second nucleic acid are the same. In another embodiment, the preMS encodes the fusion protein. In yet another embodiment, the first nucleic acid comprises a promoter operably linked to the open reading frame, and the second nucleic acid comprises a promoter operably linked to the preMS encoding sequence. In yet another embodiment, the open reading frame and the preMS encoding sequence are operably linked to a promoter.

The present invention further provides a method of making a DART, comprising growing or culturing a host cell comprising a first nucleic acid and a second nucleic acid, under conditions that allow the formation of a DART, the first nucleic acid comprising an open reading frame encoding a fusion protein. The fusion protein can comprise, for example, a Linkage Polypeptide covalently linked to a Molecular Point. The second nucleic acid can comprise, for example, a sequence encoding a preMS.

The present invention further provides a method of making a DART, comprising contacting a preMS with an autocatalytic Linkage Polypeptide covalently linked to a Molecular Point, under conditions that allow the formation of a DART.

The present invention further provides a method of making a DART, comprising contacting a preMS with (i) an Linkage Polypeptide covalently linked to a Molecular Point, the Linkage Polypeptide being a substrate for a transcatalytic linking protein, and (ii) a transcatalytic linking protein, the contacting being under conditions that allow the formation of a DART.

The present invention further provides a method of making a DART, comprising contacting a preMS with (i) a Linkage Polypeptide covalently linked to a Molecular Point, the Linkage Polypeptide being a substrate for a trans-complementary linking protein, and (ii) a trans-complementary linking protein, the contacting being under conditions that allow the formation of a DART.

The present invention also provides a DART library comprising a plurality of DARTs, each DART comprising a Molecular Shaft covalently linked to a Linkage Polypeptide covalently linked to a Molecular Point. In one embodiment, each DART species in the library comprises a different Molecular Point. In another embodiment, each DART species in the library comprises a different Molecular Shaft.

The present invention yet further provides a DART library comprising at least 10 DART species, at least 100 DART species, at least 1,000 DART species, at least 10,000 DART species, or at least 100,000 DART species.

The present invention provides a DART library comprising a plurality of DARTs, wherein the Molecular Shafts of the DART species encode a fusion polypeptide comprising its corresponding Linkage Polypeptide and Molecular Point. The present invention yet further provides a DART library comprising a plurality of DARTs, wherein the DARTs are self-referential.

The present invention yet further provides a DART library comprising a plurality of DARTs, the library being an in vivo library expressed by a population of cells. In a specific embodiment, each cell in the population expresses on average a single DART species. In one embodiment, the cells are eukaryotic cells. In another embodiment, the cells are prokaryotic cells. In a typical embodiment, expression of the in vivo library is under the control of an inducible promoter. The in vivo library can be a DNA DART library, an RNA DART library or a DNA/RNA hybrid DART library.

The present invention yet further provides a DART library comprising a plurality of DARTs, the library being an in vitro library. In one embodiment, an in vitro library comprises an array of DARTs immobilized on a surface of a solid phase. In one mode of the embodiment, each DART species is situated at a known location on the surface of the solid phase.

The present invention yet further provides an in vitro DART library which is a DNA DART library, an RNA DART library, or a DNA/RNA hybrid DART library. The present invention yet further provides an in vitro DART library in which the DARTs are purified.

The present invention also provides a DART library in which the DARTs comprise autocatalytic Linkage Polypeptides. In another embodiment, the invention provides a DART library in which the DARTs comprise non-autocatalytic Linkage Polypeptides. In one mode of the embodiment, the non-autocatalytic Linkage Polypeptides are substrates for a transcatalytic linking protein. In another mode of the embodiment, the non-autocatalytic Linkage Polypeptides are substrates for a trans-complementary linking protein.

The present invention yet further provides a DART library in which each DART species comprises a Molecular Shaft encoding a different mutant of a known protein. The present invention yet further provides a DART library in which each DART species comprises a Molecular Point encoding a different mutant of a known protein.

The present invention also provides a method of joining a first nucleic acid to a second nucleic acid, the second nucleic acid comprising a Recognition Sequence Motif. The method can comprise contacting a DART, the DART comprising (i) a Molecular Shaft comprising the first nucleic acid and (ii) a Linkage Polypeptide comprising a domain that recognizes the Recognition Sequence Motif, with the second nucleic acid under conditions that allow the first nucleic acid to be joined to the second nucleic acid. In one embodiment, the Linkage Polypeptide is an autocatalytic Linkage Polypeptide. In another embodiment, the Linkage Polypeptide is a non-autocatalytic Linkage Polypeptide. In one mode of the embodiment, the non-autocatalytic Linkage Polypeptides is a substrate for a transcatalytic linking protein. In another mode of the embodiment, the non-autocatalytic Linkage Polypeptide is a substrate for a trans-complementary linking protein.

In anther aspect, DARTboards are provided comprising an affinity substrate comprising one or more Capture Molecular Targets immobilized on the surface of a solid substrate. Typically, at least one Capture Molecular Target is bound to a DART, the DART. In one embodiment, the DARTboard comprises a plurality of Capture Molecular Targets. In one mode of the embodiment, the Capture Molecular Targets are immobilized on the surface as an array. The array can be a microarray. Typically, each Capture Molecular Target is situated at a known location on the surface of the solid phase.

The present invention also provides a DARTboard comprising a plurality of Capture Molecular Targets immobilized in an array at a density of, for example, greater than 60 Capture Molecular Targets per 1 cm².

The present invention further provides a DARTboard comprising a plurality of Capture Molecular Targets immobilized on an array, the array comprising at least 10 different species of Capture Molecular Targets, at least 100 different species of Capture Molecular Targets, at least 1,000 different species of Capture Molecular Targets or at least 10,000 different species of Capture Molecular Targets.

The present invention also provides a DARTboard in which the Capture Molecular Target is a nucleic acid. In one embodiment, the nucleic acid comprises an identification tag. In another embodiment, at least one Capture Molecular Target bound to a DART comprises a nucleic acid sequence that is complementary to a nucleic acid sequence present in the Molecular Shaft of the DART.

The present invention further provides a DARTboard in which the Capture Molecular Target is a polypeptide, such as, for example, an antibody.

A DARTboard can also include an affinity substrate comprising one or more Capture Molecular Targets immobilized on the surface of a solid substrate. Typically, at least one Capture Molecular Target is bound to a DART. The DARTboard also includes at least one non-DART molecule bound to the Molecular Point, Molecular Shaft and/or Linkage Polypeptide of a DART bound to a Capture Molecular Target. In one embodiment, the non-DART molecule is a disease-associated molecule. In another embodiment, the non-DART molecule is a Probe Molecular Target. In yet another embodiment, the non-DART molecule is a small molecule. The non-DART molecule can be covalently or non-covalently bound to the Molecular Point Molecular Shart, or the Linkage Polypeptide.

The present invention yet further provides a DARTboard comprising a plurality of Capture Molecular Targets bound to a plurality of DARTs. In one embodiment, the DARTs are obtained from prokaryotic cells. In another embodiment, the DARTs are obtained from eukaryotic cells. In certain specific embodiments, the cells had been exposed to a drug or subjected to conditions that lead to post-translational modifications prior to obtaining the DARTs.

In another aspect, methods for reducing the expression of a target nucleic acid in a cell, are provided. The methods generally include introducing into the cell one or more nucleic acids encoding an RNase DART, the RNase DART comprising (i) a Molecular Shaft comprising a nucleic acid sequence that is complementary to the target nucleic acid and (ii) a Molecular Point comprising an RNase that selectively cleaves double stranded nucleic acid sequences in which one or both strands are RNA. The cell can be grown under conditions that allow DART formation. The RNase DART bind to and cleave the target nucleic acid. In one embodiment, the RNase DART is self-referential. In another embodiment, the target nucleic acid is an RNA molecule. In one mode of the embodiment, the RNA molecule encodes a disease-associated molecule. In another mode of the embodiment, the RNA molecule is the genome of a single stranded RNA virus. In another embodiment, the target nucleic acid is a DNA molecule. In one mode of the embodiment, the DNA molecule is the genome of a single stranded DNA virus, or a retroviral cDNA. In a typical embodiment, the RNase is RNase H.

In another aspect, the present invention provides a method for reducing the expression of a target nucleic acid in a cell. The method generally includes expressing an RNase DART in the cell. The RNase DART comprises (i) a Molecular Shaft comprising a nucleic acid sequence that is complementary to the target nucleic acid and (ii) a Molecular Point comprising an RNAse that selectively cleaves double stranded nucleic acid sequences in which one or both strands are RNA. The RNase DART can bind to and cleave the target nucleic acid. In one embodiment, the RNase DART is self-referential. In another embodiment, the target nucleic acid is an RNA molecule. In one mode of the embodiment, the RNA molecule encodes a disease-associated molecule. In another mode of the embodiment, the RNA molecule is the genome of a single stranded RNA virus. In another embodiment, the target nucleic acid is a DNA molecule. In one mode of the embodiment, the DNA molecule is the genome of a single stranded DNA virus. In another mode of the embodiment, the DNA molecule is a retroviral cDNA. In a typical embodiment, the RNase is RNase H.

The present invention also provides a method for detecting a disease-associated molecule in a biological sample. The method generally includes (a) contacting the sample with a diagnostic DART under conditions in which the diagnostic DART binds to the disease-associated molecule; and (b) detecting whether the diagnostic DART is bound to the disease-associated molecule in the biological sample, thereby indicating whether the disease-associated molecule is present in the biological sample. In one embodiment, the diagnostic DART comprises a detectably-labeled Molecular Point. In another embodiment, the diagnostic DART comprises a detectably-labeled Molecular Shaft. The detectable label can be a fluorescent label. In yet another embodiment, the diagnostic DART comprises a Molecular Point, the Molecular Point comprising an antibody that binds to the disease-associated molecule. The disease-associated molecule can b, for example, a bacterial antigen, a viral antigen, a protozoal antigen, a parasitic antigen or a tumor-associated antigen.

Detection of a diagnostic DART can be achieved, for example, by affinity purifying the diagnostic DART and determining whether the disease-associated molecule is bound to the diagnostic. DART, for example, by purification on an affinity substrate, thereby forming a DARTboard. Determining whether the disease-associated molecule is bound to the diagnostic DART can achieved by (i) contacting the DARTboard with a labeled antibody that binds to the disease-associated molecule; and (ii) determining whether the labeled antibody is bound to the DARTboard.

In another aspect, the present invention provides a DART probe comprising a Molecular Shaft covalently linked to a Linkage Polypeptide covalently linked to a Molecular Point, wherein the Molecular Shaft or the Molecular Point comprises a detectable label. In one embodiment, the Molecular Shaft comprises a detectable label. In one mode of the embodiment, the detectable label is a fluorescent moiety, such as, for example, rhodamine, fluorescein, Cy3 or Cy5. In another mode of the embodiment, the detectable label is a radiolabel. One or more nucleotides in the Molecular Shaft can comprise the fluorescent moiety or radiolabel. In another embodiment, the Molecular Point comprises a detectable label. In one mode of the embodiment, the Molecular Point comprises Green Flourescent Proteins.

The present invention yet further provides a method for generating a first DART comprising a first Molecular Shaft covalently linked to a first Linkage Polypeptide. The first Molecular Shaft comprises a nucleic acid sequence of a second Molecular Shaft and a nucleic acid sequence of a third Molecular Shaft. The third Molecular Shaft comprises a Recognition Sequence Motif of a second Linkage Polypeptide. The method comprising: contacting a second DART comprising the second Molecular Shaft and the second Linkage Polypeptide with a third DART comprising the third Molecular Shaft under conditions that the first DART is formed.

The present invention yet further provides a method for detecting an interaction between a first DART and a second DART, comprising: (a) contacting a first DART with a second DART, wherein (i) the first DART comprises a first Molecular Point covalently linked to a first Linkage Polypeptide covalently linked to a first Molecular Shaft, (ii) the second DART comprises a second Molecular Point covalently linked to a second Linkage Polypeptide covalently linked to a second Molecular Shaft. The second Molecular Shaft comprises a 5′ or 3′-terminal Recognition Sequence Motif recognized by the first Linkage Polypeptide. The contacting is typically performed under a first condition that allows the interaction between the first DART and the second DART, thereby forming a DART complex. The DART complex can be subjected to a second condition that, in the presence but not absence of an interaction between the first DART and the second DART, allows the formation of a covalent bond between the first Molecular Shaft and the Recognition Sequence Motif of the second Molecular Shaft, thereby allowing the formation of a progeny DART. The progeny DART typically comprises the second Molecular Point covalently linked to the second Linkage Polypeptide covalently linked to the second Molecular Shaft covalently linked to the first Molecular Shaft. The progeny DART can be detected. In one embodiment, the first and second conditions are the same; in a more typical embodiment, the first and second conditions are different.

The present invention yet further provides a method for detecting an interaction between a first DART and a second DART, comprising (a) contacting a first DART with a second DART. The first DART comprises a first Molecular Point covalently linked to a first Linkage Polypeptide covalently linked to a single-stranded first Molecular Shaft, the first Molecular Shaft comprising a first Complementary Sequence Tail. The second DART comprises a second Molecular Point covalently linked to a second Linkage Polypeptide covalently linked to a second Molecular Shaft, wherein the single-stranded second Molecular Shaft comprises a second Complementary Sequence Tail. The second Complementary Sequence Tail is typically complementary to the first Complementary Sequence Tail. The contacting is typically performed under a first condition that allows the interaction between the first DART and the second DART, thereby forming a DART complex. The DART complex is typically subjected to a second condition that, in the presence but not absence of an interaction between the first DART and the second DART, allows the first Complementary Sequence Tail to hybridize with the second Complementary Sequence Tail, thereby forming a hybridized DART complex. The hybridized DART complex can be contacted, for example, with a polymerase under conditions that allow for the extension of the Complementary Sequence Tail duplex; thereby forming a first progeny DART and a second progeny DART. The progeny of the first DART typically comprise the Molecular Shaft of the first DART covalently linked to nucleic acid sequences complementary to those present in the Molecular Shaft of the second DART. The progeny of the second DART typically comprise the Molecular Shaft of the second DART covalently linked to nucleic acid sequences complementary to those present in the Molecular Shaft of the first DART. The first progeny DART and/or the second progeny DART can be detected. In one embodiment, the first and second conditions are the same; in another embodiment, the first and second conditions are different. In certain embodiments, the first DART or the second DART is immobilized on a solid surface, for example by means of an affinity substrate, e.g., a Molecular Target of the first or second DART.

The present invention also provides a method for detecting an interaction between a DART and a Molecular Target, optionally immobilized on a solid surface. The method generally comprises: (a) contacting a DART with a Molecular Target, wherein the DART comprises a Molecular Point covalently linked to a Linkage Polypeptide covalently linked to a Molecular Shaft. The contacting is typically performed under a first condition that allows the interaction between the DART and the Molecular Target, thereby forming a DART-Molecular Target complex. The Molecular Target (b) is contacted with Identity nucleic acid, wherein the Identity nucleic acid comprises a Recognition Sequence Motif of the Linkage Polypeptide and an Identity sequence. The DART-Molecular Target complex can be subjected to a second condition that, in the presence but not absence of a DART-Molecular Target complex, allows the formation of a covalent bond between the Molecular Shaft and the Recognition Sequence Motif of the Identity nucleic acid, thereby forming a ligated DART-Molecular Target complex. The ligated DART-Molecular Target can be detected.

In one embodiment, the detecting comprises subjecting the ligated DART-Molecular Target complex to a PCR reaction with primers corresponding to the Molecular Shaft and the Identity sequence, and detecting whether a PCR product is formed. Where the Molecular Target is immobilized on a solid surface, the Identity nucleic acid can be immobilized on the solid phase prior to contacting the DART with the Molecular Target. In other embodiments, for example where Molecular Target is not immobilized on a solid surface, step (a) typically precedes step (b). In an embodiment of this configuration, the second condition comprises, for example, contacting the DART-Molecular Target complex with the Identity nucleic acid.

The present invention also provides a method for detecting an interaction between a DART and Molecular Target which is optionally immobilized on a solid surface. The method generally comprises: (a) contacting a DART with a Molecular Target, wherein the DART comprises a Molecular Point covalently linked to a Linkage Polypeptide covalently linked to a single-stranded Molecular Shaft, the Molecular Shaft comprising a single-stranded first TAG sequence. The contacting is typically performed under a first condition that allows the interaction between the DART and the Molecular Target, thereby forming a DART-Molecular Target complex. The method further includes: (b) placing the Molecular Target in the proximity of an Identity nucleic acid (e.g. contacting), wherein the Identity nucleic acid comprises a single-stranded second TAG sequence and an Identity sequence; and (c) contacting the DART-Molecular Target complex with a Matchmaker nucleic acid. The Matchmaker nucleic acid typically comprises single-stranded regions of complementarity to the first and second TAG sequences. The method also includes (d) subjecting the DART complex to a second condition that, in the presence but not absence of a DART-Molecular Target complex, allows the first TAG sequence to indirectly hybridize with the second TAG sequence via the Matchmaker nucleic acid, thereby forming a hybridized DART-Molecular Target complex; (e) contacting the hybridized DART-molecular complex with a nucleic acid ligase under conditions that allow for ligation of the TAG sequences to the Matchmaker nucleic acid, thereby forming a ligated DART-Molecular Target complex; and (f) detecting the ligated DART-Molecular Target complex.

In one embodiment, the detecting comprises subjecting the ligated DART-Molecular Target complex to a PCR reaction with primers corresponding to the Molecular Shaft and the Identity sequence, and detecting whether a PCR product is formed. Where the Molecular Target is immobilized on a solid surface, step (b) can precede step (a), for example by immobilizing the Identity nucleic acid on the solid phase prior to contacting the DART with the Molecular Target. In other embodiments, for example where Molecular Target is not immobilized on a solid surface, step (a) typically precedes step (b). In an embodiment of this configuration, the second condition comprises, for example, contacting the DART-Molecular Target complex with the Identity nucleic acid. In other embodiments, step (b) and step (c) can be concurrent. In yet other embodiments, step (b) can precede step (c) or step (c) can precede step (b).

In another aspect, the present invention provides a method for detecting an interaction between a first DART a second DART. The method generally comprise: (a) contacting a first DART with a second DART. The first DART typically comprises a first Molecular Point covalently linked to a first Linkage Polypeptide covalently linked to a single-stranded first Molecular Shaft, the first Molecular Shaft comprising a single-stranded first TAG sequence. The second DART typically comprises a second Molecular Point covalently linked to a second Linkage Polypeptide covalently linked to a single-stranded second Molecular Shaft, the second Molecular Shaft comprising a single-stranded second TAG sequence. The contacting is typically performed under a first condition that allows the interaction between the first DART and the second DART, thereby forming a DART complex. The method also includes (b) contacting the first DART with a Matchmaker nucleic acid, wherein the Matchmaker nucleic acid comprises single-stranded regions of complementarity to the first and second TAG sequences; and (c) subjecting the DART complex to a second condition that, in the presence but not absence of a DART complex, allows the first TAG sequence to indirectly hybridize with the second TAG sequence via the Matchmaker nucleic acid, thereby forming a hybridized DART complex. The method further includes (e) contacting the hybridized DART complex with a nucleic acid ligase under conditions that allow for ligation of the TAG sequences to the Matchmaker nucleic acid, thereby forming a ligated DART complex; and (f) detecting the ligated DART complex. The complex can be detected, for example, by subjecting the ligated DART complex to a PCR reaction with primers corresponding to the first Molecular Shaft and the second Molecular Shaft, and detecting whether a PCR product is formed. In one embodiment, the first DART is immobilized on a solid phase, such as, for example, a DARTboard. In another embodiment of this method step (b) can precede step (a). In yet other embodiments, step (a) can precede step (b); step (b) and step (c) can be concurrent; or step (b) can precede step (c).

In anther aspect, the present invention provides a method for identifying a mutant protein that possesses a desired functional property, comprising mutagenizing a population of nucleic acids encoding the protein; and generating a population of nucleic acids comprising the mutagenized population. The nucleic acids encoding a fusion protein comprising a Linkage Polypeptide and a Molecular Point comprising a mutant protein. A DART library is generated from the population of mutogenized nucleic acids. The DART library is screened to identify a DART that possess the desired functional property, thereby identifying a mutant protein that possesses the desired functional property.

The present invention also provides a method for identifying a mutant protein that possesses a desired functional property, comprising (a) generating a DART library, wherein the DART species have a Molecular Points that are different mutants of the protein; and (b) screening the DART library to identify a DART that possess the desired functional property, thereby identifying a mutant protein that possesses the desired functional property.

In another aspect, a method is provided for making a Modular DART Assembly, comprising contacting a first DART and a second DART. The first DART comprises a first Molecular Point covalently linked to a first Linkage Polypeptide covalently linked to a first Molecular Shaft. The second DART comprises a second Molecular Point covalently linked to a second Linkage Polypeptide covalently linked to a second Molecular Shaft. The DARTs are contacted under conditions that allow the formation of a Modular DART assembly. In one embodiment, the first DART and the second DART in the Modular DART Assembly are complexed through a covalent or non-covalent interaction between the first Molecular Point and the second Molecular Point. In another embodiment, the first DART and the second DART in the Modular DART Assembly are complexed through a covalent or non-covalent interaction between the first Molecular Shaft and the second Molecular Shaft. In yet another embodiment, the first DART and the second DART in the Modular DART Assembly are complexed through a covalent or non-covalent interaction between the first Molecular Point and the second Molecular Shaft.

The present invention also provides a method for Modular DART Assembly comprising a first DART and a second DART, wherein (i) the first DART comprises a first Molecular Point covalently linked to a first Linkage Polypeptide covalently linked to a first Molecular Shaft, and wherein (ii) the second DART comprises a second Molecular Point covalently linked to a second Linkage Polypeptide covalently linked to a second Molecular Shaft. In one embodiment, the first DART and the second DART in the Modular DART Assembly are complexed through a covalent or non-covalent interaction between the first Molecular Point and the second Molecular Point. In another embodiment, the first DART and the second DART in the Modular DART Assembly are complexed through a covalent or non-covalent interaction between the first Molecular Shaft and the second Molecular Shaft. In yet another embodiment, the first DART and the second DART in the Modular DART Assembly are complexed through a covalent or non-covalent interaction between the first Molecular Point and the second Molecular Shaft.

The present invention yet further provides a composition comprising (a) a DART comprising a Molecular Shaft covalently linked to a Linkage Polypeptide covalently linked to a Molecular Point; and (b) a Molecular Target bound to the DART. In one embodiment, the Molecular Target is bound to the first Molecular Shaft. In another embodiment, the Molecular Target is bound to the first Molecular Point. In yet another embodiment, the Molecular Target is bound to the first Linkage Polypeptide.

The present invention also provides a composition comprising (a) a DART, the DART comprising a Molecular Shaft covalently linked to a Linkage Polypeptide covalently linked to a Molecular Point; and (b) a Molecular Target bound to the DART, in which the Molecular Target comprises a nucleic acid. In one embodiment, the nucleic acid is bound to the Molecular Shaft. In one mode of the embodiment the nucleic acid is hybridized to the Molecular Shaft. In other embodiments, the Molecular Target is bound to the Molecular Point or the Linkage Polypeptide. In some embodiments, the nucleic acid can be at least a portion of a second Molecular Shaft of a second DART.

In another aspect, at least a portion of the present invention provides a composition comprising (a) a DART, a Molecular Target bound to the DART, in which the Molecular Target comprises a polypeptide. In one embodiment, the polypeptide is bound to a Molecular Shaft. In other embodiments, the polypeptide is bound to a Molecular Point or a Linkage Polypeptide of the DART. The polypeptide can be, for example, a second Molecular Point or Linkage Polypeptide of a second DART.

The present invention yet further provides a composition comprising (a) a DART, and (b) a Molecular Target bound to the DART, in which the Molecular Target is not a DART. In various embodiments, the Molecular Target is covalently or non-covalently attached to the Molecular Shaft, to the Molecular Point or to the first Linkage Polypeptide. The Molecular Target can be, for example, a small molecule such as a drug.

The present invention also provides a DART molecular complex, comprising a first DART bound to an affinity substrate. The affinity substrate can be, for example, a Molecular Target. In one embodiment, the affinity substrate can be immobilized on a solid surface, such as, for example, an agarose bead, a polystyrene bead, a magnetic bead, a glass slide, a glass bead, a silicon wafer, a microtiter plate, a nitrocellulose membrane, a nylon membrane, or a PVDF membrane. In one embodiment, the first DART comprises a first TAG sequence. In a typical mode of the embodiment, the solid surface further comprises a nucleic acid immobilized to the solid surface, the nucleic acid comprising a second TAG sequence. In another mode of the embodiment, the first DART is bound to a second DART, which second DART comprises a second TAG sequence. In certain embodiments, the first TAG and the second TAG indirectly hybridize to one another through a Matchmaker nucleic acid, which Matchmaker nucleic acid comprises single stranded regions of complementarity to the first and second TAGs, respectively.

The present invention also provides a DART molecular complex, comprising a first DART and a second DART. In one embodiment, the first DART is immobilized on a solid surface such as, for example, an agarose bead, a polystyrene bead, a magnetic bead, a glass slide, a glass bead, a silicon wafer, a microtiter plate, a nitrocellulose membrane, a nylon membrane, a PVDF membrane, or a DARTboard. In one embodiment, the first DART comprises a first TAG sequence; optionally, the second DART comprises a second TAG sequence. In an embodiment in which the first DART and the second DART comprise a first and second TAG sequence, respectively, the first TAG and the second TAG are indirectly hybridized to one another by a Matchmaker nucleic acid, which Matchmaker nucleic acid comprises single stranded regions of complementarity to the first and second TAG sequences, respectively.

In another aspect, the present invention provides a kit comprising in one or more containers, at least one DART, the DART comprising a Molecular Shaft covalently linked to a Linkage Polypeptide covalently linked to a Molecular Point. In one embodiment, the Molecular Shaft is RNA. In another embodiment, the Molecular Shaft is DNA. In yet another embodiment, the Molecular Shaft is a DNA/RNA hybrid. The Molecular Shaft can be single stranded, double stranded and/or partially double stranded. In one embodiment, the Molecular Shaft can be detectably labeled. In another embodiment, the Molecular Point can be detectably labeled. In yet another embodiment, the DART can be a diagnostic DART or a DART probe.

The present invention also provides a kit comprising in one or more containers a plurality of DARTs. In one embodiment, the DARTs are immobilized on the surface of a solid surface.

The present invention yet further provides a kit comprising in one or more containers a first nucleic acid and a second nucleic acid, wherein the first nucleic encodes a fusion protein comprising a Molecular Point and a Linkage Polypeptide and the second nucleic acid encodes a preMS. The first nucleic acid and the second nucleic acid can be the same or different.

The present invention also provides a kit comprising in one or more containers a first nucleic acid and a second nucleic acid, wherein the first nucleic comprises (i) a sequence encoding a Linkage Polypeptide and (ii) at least one recognition site for a restriction enzyme 3′- or 5′-to the sequence encoding the Linkage Polypeptide; and wherein the second nucleic acid encodes a preMS. In one embodiment, the first nucleic and the second nucleic acid are the same.

### BRIEF DESCRIPTION OF THE DRAWINGS

FIGS. 1A-C depict examples of a DART, a DART library and an example of a reversible, autocatalytic, LP-mediated covalent linkage reaction, respectively. Molecular Points (MPs), Linkage Polypeptides (LPs), and nucleic acids are designated by triangles, rectangles (or squares), and straight lines, respectively. Recognition Sequences Motifs (RSs) in nucleic acid components are designated by ‘wavy’ lines. Colons between two components denote a non-covalent interaction. A straight line connecting two components denotes a covalent interaction. Unless otherwise indicated, these conventions are used throughout the figures.

FIG. 2 depicts an example of DARTex. The parent DARTs (DARTex parents) are designated MP.Nᵢ-LP₁-MS.Nᵢ and MP.Nⱼ-LP₂-MS.Mⱼ. The Recognition Sequence Motif is designated by wavy lines. Linkage Polypeptides 1 and 2 are designated by shaded and unshaded boxes, respectively. Molecular Point interactions among DARTex parents facilitate nucleic acid exchange between DARTs, as depicted in the middle of the figure. The progeny that result from the strand exchange are depicted at the bottom of the figure.

FIG. 3 depicts an example of DARTdance. Molecular Point interactions among DARTdance parents stimulate, under the appropriate conditions, duplex formation between corresponding complementary Molecular Shaft Complementary Sequence Tag (CST) components. This duplex DNA can then be employed to prime an extension reaction that can generate DARTdance progeny. The symbols used in this Figure as explained in the legend to FIG. 1. Four parallel slashes indicates a CST.

FIG. 4 depicts an example of DARTdance (Matchmaker variant) on an affinity substrate. Molecular Point interactions among DARTdance parents permit subsequent binding to a Matchmaker nucleic acid via TAG sequences. The Matchmaker can then be ligated to the molecular shafts of the DARTs to yield the DARTdance progeny. The Matchmaker is designated in the middle right portion of the figure as parallel composite wavy and straight lines. The single stranded ends of the Matchmaker are designated by shaded and unshaded boxes at the ends of the Matchmaker. The affinity substrate is designated as a curved line with a receptor symbol. The shaded circle of the Molecular Point of DARTdance parent MP.Nⱼ-LP-MS.Nⱼ-TAG.N binds the receptor of the affinity substrate.

FIG. 5 depicts an example of DARTdance (Matchmaker variant) with a Molecular Target on an affinity substrate. Each different Molecular Target is bound to an affinity substrate in close proximity to its corresponding identity nucleic acid. Molecular Point binding to a Molecular Target on an affinity substrate can permit subsequent binding to a Matchmaker nucleic acid via TAG sequences present in the DART's Molecular Shaft and the identity nucleic acid bound to the affinity substrate. The Matchmaker is then ligated to the Molecular Shaft and to the identify nucleic acid. Ligation products are typically analyzed by PCR amplification and sequencing. The identity sequence is depicted as a carved line between the affinity substrate and the TAG.N sequence (a shaded box.).

FIG. 6 depicts an example of DARTex reaction analyses. A nucleic acid array harboring ordered and indexed sequences complementary to appropriate sequences present in the Molecular Shafts of expected DARTex progeny is depicted. This array may be employed as shown to rapidly and efficiently resolve, identify, and quantify DARTex progeny. MT₃ and MT₇ designate Molecular Tags.

### DETAILED DESCRIPTION OF THE INVENTION

Definitions

Prior to setting forth the invention in more detail, it may be helpful to a further understanding thereof to set forth definitions of certain terms as used hereinafter. Unless defined otherwise, all technical and scientific terms used herein have the same meaning as commonly understood by one of ordinary skill in the art to which this invention belongs. Only exemplary methods and materials are described, and any methods and materials similar to those described herein can be used in the practice of the present invention. For purposes of the present invention, the following terms are defined below.

The terms “polynucleotide” and “nucleic acid” refer to a polymer composed of a multiplicity of nucleotide units (ribonucleotide or deoxyribonucleotide or related natural or synthetic structural variants) linked via diester, such as phosphodiester and thiodiester, bonds. A polynucleotide or nucleic acid can be of substantially any length, typically from about six (6) nucleotides to about 10⁹ nucleotides or larger. Polynucleotides and nucleic acids include RNA, cDNA, genomic DNA, synthetic forms, and mixed polymers, both sense and antisense strands, and can also be chemically or biochemically modified or can contain non-natural or derivatized nucleotide bases, as will be readily appreciated by the skilled artisan. Such modifications include, for example, labels, methylation, substitution of one or more of the naturally-occurring nucleotides with an analog, internucleotide modifications such as uncharged linkages (e.g., methyl phosphonates, phosphotriesters, phosphoamidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The term “oligonucleotide” refers to a polynucleotide of from about six (6) to about one hundred (100) nucleotides or more in length. Thus, oligonucleotides are a subset of polynucleotides. Oligonucleotides can be synthesized, for example, on an automated oligonucleotide synthesizer (for example, those manufactured by Applied BioSystems (Foster City, Calif. )) according to specifications provided by the manufacturer.

The term “primer” as used herein refers to a polynucleotide, typically an oligonucleotide, whether occurring naturally, as in an enzyme digest, or whether produced synthetically, which acts as a point of initiation of polynucleotide synthesis when used under conditions in which a primer extension product is synthesized. A primer can be single-stranded, partially double-stranded or double-stranded.

The phrase “corresponds to,” in the context of polynucleotides, means that one polynucleotide sequence is identical to at least a portion of another polynucleotide sequence. In contrast, the term “complementary to” means that the sequence is identical to at least a portion of the complement of another polynucleotide sequence. For purposes of illustration only, the nucleotide sequence 5′-ATACT-3′ corresponds to a reference sequence 5′-ATACT-3′ and is complementary to a reference sequence 5′-AGTAT-3′. The corresponding nucleic acid for a polypeptide is a nucleic acid that encodes the polypeptide.

The term “polypeptide” refers to a polymer of amino acids and its equivalent and does not refer to a specific length of the product; thus, peptides, oligopeptides and proteins are included within the definition of a polypeptide. A “fragment” refers to a portion of a polypeptide having typically at least 10 contiguous amino acids, more typically at least 20, still more typically at least 50 contiguous amino acids. A derivative is a polypeptide having conservative amino acid substitutions, as compared with another sequence. Derivatives further include, for example, glycosylations, acetylations, phosphorylations, and the like. Further included within the definition of“polypeptide” are, for example, polypeptides containing one or more analogs of an amino acid (e.g., unnatural amino acids, and the like), polypeptides with substituted linkages as well as other modifications known in the art, both naturally- and non-naturally occurring. Ordinarily, such polypeptides will be at least about 50% identical to the reference amino acid sequence, typically in excess of about 90%, and more typically at least about 95% identical.

The term “isolated” refers to a molecule (e.g., a nucleic acid or polypeptide or combination thereof that, by the hand of people, exists apart from its native environment and is therefore not a product of nature. An isolated nucleic acid or polypeptide molecule can exist in a purified form or can exist in a non-native environment such as, for example, a recombinant host cell.

The terms “amino acid” or “amino acid residue”, as used herein, refer to naturally-occurring L amino acids or to D amino acids as described further below. The commonly used one- and three-letter abbreviations for amino acids are used herein (see, e.g., Alberts et al., Molecular Biology of the Cell, Garland Publishing, Inc., New York (3d ed. 1994)).

The terms “covalent bond” or “covalent linkage” refer to a bond in which a pair of electrons is shared between atoms. A covalent bond is distinct from an ionic bond or a hydrogen bond.

The term “fusion polypeptide” refers to two polypeptide chains that are covalently linked via a peptide bond.

The term “antibody” refers to a polypeptide substantially encoded by an immunoglobulin gene or immunoglobulin genes, or fragments thereof, that specifically binds and recognizes an analyte (antigen). Immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

Antibodies include, for example, intact immunoglobulins and antigen-binding fragments. Thus, the term antibody, as used herein, also includes antibody fragments, such as a single chain antibody, an antigen binding F(ab′)₂ fragment, an antigen binding Fab′ fragment, an antigen binding Fab fragment, an antigen binding Fv fragment, a single heavy chain or a chimeric antibody. Such antibodies can be produced, for example, by the modification of whole antibodies or synthesized de novo using recombinant DNA methodologies.

The term “Dynamic Action Reference Tool” or “DART” refers to molecule having a Molecular Shaft, a Linkage Polypeptide and a Molecular Point. The Molecular Shaft is covalently linked to the Linkage Polypeptide which is covalently linked to the Molecular Point.

The term “Linkage Polypeptide” or “LP” refers to a polypeptide that forms a covalent bond with a nucleic acid, either as the result of an autocatalytic reaction (i.e., the Linkage Polypeptide catalyzes the reaction which results in the covalent linkage between it and the nucleic acid) or as a result of a non-autocatalytic reaction (i.e., the reaction that results in the covalent linkage between the Linkage Polypeptide and nucleic acids cannot occur in the absence of additional polypeptides), or as a result of a trans-catalytic reaction (i.e., the reaction that results in the covalent linkage between the Linkage Polypeptide and nucleic acid is catalyzed by a second polypeptide). (The additional transcomplementing polypeptides may or may not catalyze the linkage reaction.) The formation of a covalent bond can be reversible or irreversible, as will be described in below. In a typical autocatalytic reaction, the Linkage Polypeptide recognizes and binds to a Recognition Sequence Motif in a nucleic acid and participates in the formation of a covalent linkage between a residue of the Linkage Polypeptide and a nucleotide of the nucleic acid. Under certain conditions, an LP may also cleave the nucleic acid containing the Recognition Sequence Motif. Linkage may also arise as a consequence of the cleavage activity of the Linkage Polypeptide and may occur prior to, concurrently with or following the formation of a covalent bond between the Linkage Polypeptide and nucleic acid. For example, a hydroxyl group on the Linkage Polypeptide (e.g., on a tyrosine, serine or threonine residue) can form a phosphodiester bond with a phosphate group on the nucleic acid (e.g., the 5′ phosphate group of the 5′ terminal nucleotide of the nucleic acid). Other linkages are also possible, as will be described below. Exemplary Linkage Polypeptides that are useful in the present invention are also described. In a typical embodiment, a Linkage Polypeptide of the present invention does not oligomerize, e.g., dimerize or multimerize, in the conditions under which a DART comprising the Linkage Polypeptide is formed, or in the conditions for which a DART comprising the Linkage Polypeptide is used. If a Linkage Polypeptide has a tendency to oligomerize, a DART comprising the Linkage Polypeptide optionally can be made with a truncated form of the Linkage Polypeptide which lacks the region of the Linkage Polypeptide responsible for oligomerization, while retaining linkage activity. Such a truncated Linkage Polypeptide is exemplified below.

The term “Recognition Sequence Motif” or “RS” refers to a nucleic acid primary sequence, secondary structure and/or tertiary structure that is specifically recognized by a Linkage Polypeptide or by a protein that catalyzes the formation of a covalent bond between the Linkage Polypeptide and a nucleic acid sequence prior to, during or subsequent to formation of a covalent linkage between the Linkage Polypeptide and the nucleic acid. The Recognition Sequence Motif can, but need not, include a site cleaved by the Linkage Polypeptide; the cleavage site can be outside the Recognition Sequence Motif. In some aspects, a Recognition Sequence Motif can be tolerant of sequence changes from the naturally-occurring Recognition Sequence Motif recognized by the Linkage Polypeptide (e.g., changes in non-essential nucleic acids and/or changes in stem-loop structures that conserve the stem-loop).

The term “Molecular Point” or “MP” refers to a non-nucleic acid molecule that is covalently linked to the Linkage Polypeptide. The Molecular Point can be linked to the amino terminus or carboxyl-terminus of the Linkage Polypeptide, although other configurations are possible. In one embodiment, the Molecular Point is not a label.

The term “Molecular Shaft” or “MS” refers to nucleic acid that is covalently linked to a Linkage Polypeptide. In certain embodiments, a Molecular Shaft comprises a nucleic acid sequence that is not naturally covalently linked to the Linkage Polypeptide in vivo (e.g., as found in nature, such as for T-DNA when the Linkage Polypeptide is VirD2). In other embodiments, a Molecular Shaft comprises a nucleic acid sequence that is not naturally covalently linked to the Linkage Polypeptide in vivo and that is not a fragment or derivative of a nucleic acid that is naturally covalently linked to the Linkage Polypeptide in vivo. In additional embodimens, a Molecular Shaft can include a nucleic acid sequence that is (a) not naturally covalently linked to the Linkage Polypeptide in vivo (e.g., T-DNA when the Linkage Polypeptide is VirD2) or (b) not a fragment or derivative of a nucleic acid that is naturally covalently linked to the Linkage Polypeptide in vivo.

In autocatalytic reactions resulting in DART formation, the Linkage Polypeptide is required or involved for the formation of the covalent bond between the nucleic acid of the Molecular Shaft and the Linkage Polypeptide. In one embodiment, the formation of a Molecular Shaft is catalyzed by the Linkage Polypeptide alone. In another embodiment, the formation of a Molecular Shaft is catalyzed by a Linkage Polypeptide and one or more additional polypeptides. In non-autocatalytic reactions resulting in DART formation, the formation of a Molecular Shaft typically requires the participation of polypeptides other than the Linkage Polypeptide. These additional transcomplementing polypeptides may or may not catalyze the linkage reaction. In trans-catalytic reactions resulting in DART formation, the formation of a Molecular Shaft is catalyzed by a protein that is not a Linkage Polypeptide (but may comprise a functional domain of an autocatalytic Linkage Polypeptide), i.e., a protein that does not become covalently linked to preMS (e.g., a nucleic acid), as will be described infra. A Molecular Shaft car, be DNA, RNA or an RNA/DNA hybrid. A Molecular Shaft also can comprise nucleotide analogs of DNA or RNA. A Molecular Shaft typically does not comprise puromycin. A Molecular Shaft can be single-stranded, double-stranded, or partially double-stranded.

The phrase “corresponds to,” in the context of a DART, indicates a relationship between the Molecular Point and Molecular Shaft.

The term “preMS molecule” refers to a nucleic acid including one or more Recognition Sequence Motifs. The preMS molecule, or a portion thereof, becomes covalently linked to the Linkage Polypeptide.

The term “postMS molecule” refers to a byproduct of DART synthesis or recombination (e.g., “DARTex”) that includes at least a portion of a preMS molecule that does not become covalently linked to a Linkage Polypeptide.

The term “species” refers to molecules that are identical to one another.

The term “library” refers to a collection of different nucleic acids, polypeptides, and the like. A library can include a collection of DARTs that have different Molecular Points, Linkage Polypeptides, and/or Molecular Shafts. A DART library typically has at least a plurality of different DARTs, or at least ten different DARTs.

The term “Purification Substrate” or “PS” refers to a solid or semi-solid surface on which molecules can be covalently or non-covalently attached or immobilized. Examples of Purification Substrates include, but are not limited to, a-arose beads, polystyrene beads, magnetic beads, glass slides or beads, silicon wafers, the wells of a microtiter plate, nitrocellulose membranes, nylon membranes, PVDF membranes, and the like. Nucleic acids, polypeptides, antibodies, non-polypeptide molecules, and the like, can be attached to or immobilized on a Purification Substrate.

The term “Molecular Target” or “MT” refers to a molecule, including a nucleic acid, protein, DART, or other molecule, that specifically binds to a DART or a complex containing one or more DARTs and/or non-DART molecules. The Molecular Target can bind to the Molecular Shaft, the Linkage Polypeptide and/or the Molecular Point of the DART. Molecular Targets can include, but are not limited to, any one or combination of, nucleic acids, polypeptides, antibodies, and/or non-polypeptide molecules.

The term “Capture Molecular Target” or “CMT” refers to a Molecular Target through which a DART is bound or coupled to a solid surface. A CMT can bind to a DART, a component of a DART, a complex of DARTs, or a complex harboring DARTs and other molecules. A Capture Molecular Target can be a component of an Affinity Substrate. A CMT is typically a protein, an antibody, a small molecule, or a nucleic acid.

The term “Probe Molecular Target” or “PMT” refers to a Molecular Target that binds to a DART, a component of a DART, or a complex of DARTs with other DARTs or other molecules, when the DART is bound to an Affinity Substrate. PMTs are generally used to detect the presence or determine the identity of a DART on a DARTboard. A PMT is typically a protein, nucleic acid, small molecule, drug, or drug-derivative, and is typically labeled.

The term “Affinity Substrate” or “AS” refers to a Molecular Target covalently or non-covalently attached to or immobilized on a Purification Substrate, also designated herein as MT-PS or MT:PS, respectively. These designations (a hyphen “-” for covalent linkage and a colon “:” for non-covalent linkage) will be used in various contexts throughout the specification.

The term “Affinity Target” or “AT” refers to a molecule or molecules that can specifically bind, covalently or non-covalently, to an Affinity Substrate. An Affinity Target can be, for example, a DART, a DART component (e.g., a Molecular Point, Linkage Polypeptide, and/or Molecular Shaft), a combination of DART components, or other molecule that is covalently or non-covalently bound to a DART. For example, an Affinity Target can be a DART that can bind to a nucleic acid (a MT) immobilized on a Purification Substrate.

The term “DARTboard” refers to an array of DARTs in which each DART is bound, covalently or non-covalently, to a Capture Molecular Target on a supported substrate, Purification Substrate, or Affinity Substrate and optionally can include DART and/or non-DART molecules not bound to Capture Molecular Targets.

The term “nuclear localization sequence”, or “NLS” refers to an amino acid sequence that facilitates localization of a polypeptide to the nucleus of a cell.

As used herein, the terms “label” or “labeled” refer to a molecule or groups of molecules which can provide a detectable signal when the label is incorporated into, or attached to, a nucleic acid, polypeptide, antibody, and the like. For example, a nucleic acid can be labeled by incorporation of one or more radiolabeled nucleotides, nucleotides attached to a luminescent or fluorescent molecule, or by incorporation of nucleotides having biotinyl moieties that can be detected by labeled avidin (e.g., streptavidin containing a fluorescent molecule or a colored molecule produced by enzymatic activity that can be detected by optical or colorimetric methods). Methods of labeling nucleic acids, polypeptides and antibodies are well known in the art. (See, e.g., Sambrook et al., Molecular Clotting, A Laboratory Manual, 3rd ed., Cold Spring Harbor Publish., Cold Spring Harbor, New York (2001); Ausubel et al., Current Protocols in Molecular Biology, 4th ed., John Wiley and Sons, New York (1999); which are incorporated by reference herein.) Examples of detectable labels include, but are not limited to, the following: radioisotopes (e.g., ³H, ¹⁴C, ³²P, ³⁵S, ¹²⁵I, ¹³¹I, and the like), fluorescent molecules (e.g., fluorescein isothiocyanate (FITC), rhodamine, phycoerythrin (PE), phycocyanin, allophycocyanin, ortho-phthaldehyde, fluorescamine, peridinin-chlorophyll a (PerCP), Cy3 (indocarbocyanine), Cy5 (indodicarbocyanine), lanthanide phosphors, and the like), enzymes (e.g., horseradish peroxidase, β-galactosidase, luciferase, and alkaline phosphatase), biotinyl groups, and the like. In some embodiments, detectable labels are attached by spacer arms of various lengths to reduce potential steric hindrance.

### DESCRIPTION OF THE SPECIFIC EMBODIMENTS

The present invention provides Dynamic Action Reference Tools, or DARTs, and methods of making and using DARTS for the isolation and analysis of nucleic acids and polypeptides, modulating biological affinities, and the like. A DART is a molecule that includes a Molecular Shaft covalently linked to a Linkage Polypeptide that is covalently linked to a Molecular Point. DARTs, and DART libraries, can be formed and manipulated in vivo or in vitro. DARTs can be purified, and portions of DARTs can be exchanged with portions of other DARTs, and assembled into higher order complexes, as more fully described below.

Dynamic Action Reference Tool (DARTS)

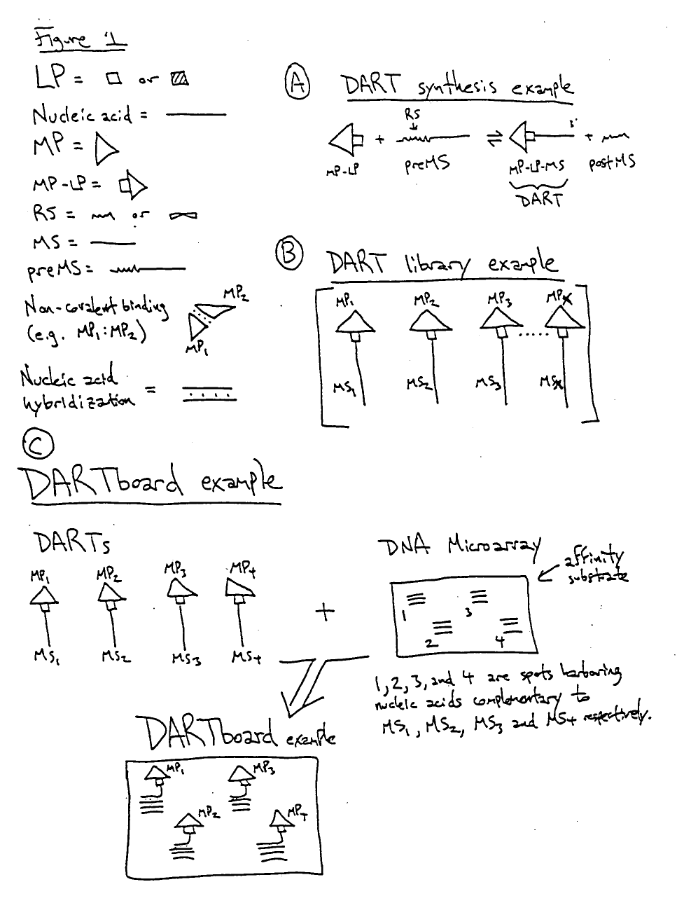

In one aspect, the present invention provides DARTs comprising a molecule that includes a Molecular Shaft covalently linked to a Linkage Polypeptide that is covalently linked to a Molecular Point. (See, e.g., FIG. 1.) The Molecular Point can be a polypeptide, including but not limited to, an antibody, and/or other non-nucleic acid molecule. Non-nucleic acid molecules may include but are not limited to carbohydrates, polysaccharides, oligosaccharides, synthetically or naturally modified carbohydrates, synthetically or naturally-modified polysaccharides, synthetically or naturally modified oligosaccharides, lipids, glycolipids, natural or synthetic modifications of these, a small organic molecule, or combinations of these.

The protein, antibody or other non-nucleic acid molecule can be of known or unknown function. For example, the Molecular Point can be a polypeptide or protein of unknown function identified by DNA sequence analysis of its encoding sequence (e.g., from a genomic DNA database, expression library and the like). A Molecular Point can also be a polypeptide or protein that is known or suspected to have a binding partner (i.e., a protein that binds to another protein, a nucleic acid or other molecule). A Molecular Point can also be a polypeptide or protein of known function, such as an antibody, that contains amino acid sequences that can be used to target the DART to a specific target molecule, intracellular location, extracellular location, and the like.

The Molecular Shaft can be a nucleic acid, such as DNA, RNA or an RNA/DNA hybrid. A “RNA/DNA hybrid” includes but is not limited to a single polymer containing RNA and DNA components. The Molecular Shaft can be single-stranded, double-stranded or partially double-stranded. The Molecular Shaft can be a gene, mRNA or other nucleic acid of known or unknown function. As will be discussed in more detail below, a Molecular Shaft can be used to target a DART to a specific target molecule, e.g., a nucleic acid complementary to the Molecular Shaft or one with which the Molecular Shaft can form a nucleic acid triplex.

A DART can have an informational relationship between the Molecular Point and the Molecular Shaft. For example, the DART is “self-referential” if the Molecular Shaft encodes the Molecular Point. Such self-referential DARTs are typically prepared by covalently coupling a MP-LP fusion or pair and a preMS molecule that encodes the MP-LP, and allowing a covalent linkage reaction to form the DART. Alternatively, the informational relationship can be non-self-referential. In this case, a MS code can be employed that provides information about the MP. Alternatively, the MS can be complementary to a nucleic acid encoding the MP or to a targeting sequence.

A DART is typically formed by a covalent linkage reaction between a preMS molecule and a Linkage Polypeptide. (See, e.g., FIG. 1.) The covalent linkage reaction forms a covalent bond between a residue of the Linkage Polypeptide and the preMS molecule, or a portion thereof. The catalytic activity to perform the covalent linkage reaction can reside in one or more of: the Linkage Polypeptide, the preMS molecule, or other molecules. The covalent linkage can be reversible or irreversible. Proteinaceous and/or non-proteinaceous cofactors may be required to facilitate DART formation (e.g., VirD1 and/or divalent magnesium ions when the Linkage Polypeptide is A. tumefaciens VirD2).

The preMS molecule and the Linkage Polypeptide typically associate in a sequence-specific manner. The Linkage Polypeptide recognizes a sequence on a preMS molecule, a Recognition Sequence Motif. The preMS molecule and Linkage Polypeptide then interact to form a covalent bond in a covalent linkage reaction. The covalent linkage reaction between the Linkage Polypeptide and the preMS molecule can remove all, part, or none of the Recognition Sequence Motif from the preMS molecule. In a typical embodiment, the Linkage Polypeptide, or a Molecular Point—Linkage Polypeptide pair, cleaves the preMS molecule within or adjacent to the Recognition Sequence Motif and forms a covalent linkage with a portion of the cleaved preMS molecule; the other portion of the preMS molecule is released as a postMS molecule. In other embodiments, the Linkage Polypeptide associates with the Recognition Sequence Motif and forms a covalent linkage with an end of the preMS molecule.

The preMS molecule can be obtained from a variety of sources, including but not limited to, in vitro synthesis (e.g., polymerase chain reaction (PCR) or other enzyme-mediated in vitro nucleic acid synthesis reactions), chemical oligonucleotide synthesis, and the like. The preMS molecule can also be prepared by in vivo synthesis, such as preparation in biological systems, including, for example, recombinant viruses, prokaryotic cells or eukaryotic cells. The preMS molecule can be single-stranded, partially double-stranded or double-stranded, and can be RNA, DNA, an RNA/DNA hybrid, and the like.

Autocatalytic Linkage Polypeptides

Suitable autocatalytic Linkage Polypeptides useful in the practice of the present invention include proteins that form a covalent linkage with nucleic acids. These proteins include those that participate in reactions mediating conjugative transfer of nucleic acids between prokaryotic cells (e.g., relaxases), transfer of T-DNA into plant cells, replication proteins of phage, viral capping proteins, recombinases, and site-specific endonucleases. The Linkage Polypeptide can be a full-length protein or a catalytically active domain that can participate in a covalent linkage reaction between itself and a preMS molecule. Linkage Polypeptides form covalent bonds with RNA, DNA and/or RNA/DNA hybrids. Some Linkage Polypeptides form covalent linkages with single-stranded nucleic acids, while others form covalent linkages with preMS molecules that are part of double-stranded nucleic acids. Certain Linkage Polypeptides can form covalent linkages with preMS molecules that are single or double-stranded nucleic acids, depending on the cofactors present in the linkage reaction.

Some Linkage Polypeptides, when acting in appropriately selected ill vitro or ill vivo systems, promote the generation of DART molecules that harbor Molecular Shaft components that are hybridized to or associated with complementary nucleic acid strands. For example, some Linkage Polypeptides from viral capping or replication proteins are of this type.

Linkage Polypeptides can not only form covalent linkages with preMS molecules that are single or double-stranded nucleic acid substrates, but also generate, in the appropriately selected in vitro or in vivo systems, DART molecules that harbor Molecular Shaft components that are not necessarily hybridized to complementary nucleic acid strands. These Linkage Polypeptides can, in the appropriately selected in vitro or in vivo systems, generate these non-hybridized DART products from double-stranded nucleic acids. The virD2 protein from the soil bacterium A. tumefaciens (and other proteins in its class) is an exemplary embodiment of this type of Linkage Polypeptide.

Examples of Linkage Polypeptides include VirD2 from A. tumefaciens (Jasper et al., 1994, Proc. Natl. Acad. Sci. USA 91:694-98; Pansegrau et al., 1993, Proc. Natl. Acad. Sci USA 90:11538-42), TraI from Escherichia coli (Pansegrau and Lanka, 1996, J. Biol. Chem. 271:13068-76), Mob A from Escherichia coli (Scherzinger et al., 1992, Nucleic Acids Res. 20:41-48; Bhattachadjee and Meyer, 1991, Nucleic Acids Res. 19:1129-37), Polio virus Vpg (Lee et al., 1977, Proc. Natl. Acad. Sci. USA 74:59-63), and Adenovirus cap protein (Salas and Vinuela, 1980, TIBS, July 1980, pp. 191-193).

In certain embodiments of the present invention, the Linkage Polypeptide is a replication protein of a single-stranded DNA bacterial virus or bacteriophage. In one embodiment, the Linkage Polypeptide is encoded by a gene A of an icosahedral bacteriophage. In certain specific modes of the embodiment, the icosahedral bacteriophage is, for example, φX174, φX, φG4, φS13, ^{SM}P2, φ186 or +PM2.

In another embodiment, the Linkage Polypeptide is encoded by a gene II of a filamentous bacteriophage. In certain specific modes of the embodiment, the filamentous bacteriophage is, for example, fd, f1, M13, ZJ/2, Ec9, AE2 or δA.

In yet another embodiment, the Linkage Polypeptide is a Xanthomonas virus replication protein. In a specific mode of the embodiment, the Xanthomonas virus is, for example, XF1.

In yet another embodiment, the Linkage Polypeptide is a Pseudomonas virus replication protein. In certain specific modes of the embodiment, the Pseudomonas virus is, for example, PF1, PF2, or PF3.

In yet another embodiment, the Linkage Polypeptide is a Vibrioparahemolyticus virus replication protein. In a specific modes of the embodiment, the Vibrioparahemolyticus virus is, for example, V6.

In yet another embodiment, the Linkage Polypeptide is a replication protein of a single-stranded DNA mycobacterial virus or bacteriophage. In a specific mode of the embodiment, the mycobacterial virus is, for example, MVL51.

In yet other embodiments, the Linkage Polypeptide is a nicking or relaxase enzyme of a bacterial plasmid. In one embodiment, the Linkage Polypeptide is a nicking or relaxase enzyme of a narrow host range plasmid. In certain specific modes of the embodiment, the narrow host range plasmid is, for example, F plasmid, R1, R100, 61B4-K98, P307, or pED208. In yet another mode of the embodiment, the Linkage Polypeptide is TraI of F plasmid.

In another embodiment, the Linkage Polypeptide is a nicking or relaxase enzyme of a broad host range plasmid. In certain specific modes of the embodiment, the broad host range plasmid is, for example, RP4, Rp1, RK2, R18, R68 or R751. In yet another mode of the embodiment, the Linkage Polypeptide is TraI of RP4.

In another embodiment, the Linkage Polypeptide is a nicking or relaxase enzyme of a mobilizable plasmid. In certain specific modes of the embodiment, the mobilizable plasmid is, for example, IncQ, RSF1010, R300B, or R1162. In yet another mode of the embodiment, the Linkage Polypeptide is MobA of IncQ.

In another embodiment, the Linkage Polypeptide is a nicking or relaxase enzyme of an R388-related plasmid. In certain specific modes of the embodiment, the R388-related plasmid is R388. In yet another mode of the embodiment, the Linkage Polypeptide is TrwC of R388.

In another embodiment, the Linkage Polypeptide is VirD2 enzyme of an A. tumefaciens Ti plasmid. In a specific mode of the embodiment, the plasmid is IncQ.

In yet other embodiments, the Linkage Polypeptide is a viral capping protein. In one embodiment, the Linkage Polypeptide is a P protein of a Hepadnavirus. In a specific mode of the embodiment, the Hepadnavirus is Hepatitis B virus.

In another embodiment, the Linkage Polypeptide is a VpG protein of a Picornavirus. In certain specific modes of the embodiment, the Picornavirus is, for example, poliovirus, apthovirus, cardiovirus, hepatitis A virus, enterovirus, rhinovirus, or coxsackievirus. In another embodiment, the Linkage Polypeptide is an adenovirus terminal protein.

In yet other embodiments of the present invention, the Linkage Polypeptide is a site-specific recombinase that has been modified to form a covalent link with a nucleic acid comprising the recognition site of the recombinase. In one embodiment, the site-specific recombinase is phage λ integrase. In another embodiment, the site-specific recombinase is CRE recombinase of phage P1. In yet other embodiment, the site-specific recombinase is mammalian RAG1 or RAG2 recombinase. In yet another embodiment, the site-specific recombinase is an integron integrase. In yet another embodiment, the site-specific recombinase is FLP recombinase of Saccharomyces cerevisiae.

In yet another embodiment of the present invention, the Linkage Polypeptide is a site-specific endonuclease that has been modified to form a covalent link with a nucleic acid comprising the recognition site of the endonuclease. In certain specific embodiments, the site-specific endonuclease is, for example, EcoRI HindIII, ClaI, BamHI, BglII, BglI, PstI, XhoI, or XbaI. In other embodiments, the site-specific endonuclease is HO endonuclease of Saccharomyces cerevisiae.

In yet another embodiment of the present invention, the Linkage Polypeptide is a polypeptide fusion containing partial or full polypeptide sequences of a Linkage Polypeptide and an accessory polypeptide factor that may participate in regulating or otherwise mediating the covalent coupling of Linkage Polypeptide sequences to nucleic acids. In one specific mode of this embodiment, the Linkage Polypeptide is a single polypeptide fusion containing partial or full polypeptide sequences of A. tumefaciens proteins virD1 and virD2 which includes linking activity.

In yet another embodiment of the present invention, the Linkage Polypeptide is a truncated or mutated form of TraI that possesses the capacity to covalently couple itself to single-stranded nucleic acids, but lacks the capacity to covalently couple itself to double-stranded (i.e., duplex) nucleic acids.

In yet other embodiments of the invention, the Linkage Polypeptide is, for example, a geminivirus replication protein, a caulimovirus replication protein, a badnavirus replication protein, a reovirus replication protein, a phytovirus replication protein, a fijivirus replication protein, an oryzavirus replication protein, a partitivirus replication protein, an alphacryptovirus replication protein, a betacryptovirus replication protein, a rhabdovirus replication protein, a nucleorhabdovirus replication protein, a bunyavirus replication protein, a topsovirus replication protein, a tenuivirus replication protein, a sequivirus replication protein, a tombusvirus replication protein, a dianthovirus replication protein, an enamovirus replication protein, an idaeovirus replication protein, a luteovirus replication protein, a machlomovirus replication protein, a marafivirus replication protein, a necrovirus replication protein, a sobemovirus replication protein, a tymovirus replication protein, an umbravirus replication protein, a bromovirus replication protein, a comovirus replication protein, a tobamovirus replication protein, a hordeivirus replication protein, a tobravirus replication protein, a furoivirus replication protein, a potexvirus replication protein, a capillovirus replication protein, a trichovirus replication protein, a carlavirus replication protein, a potyvirus replication protein, a closterovirus replication protein, a parvovirus replication protein, a baculovirus replication protein, a nudivirus replication protein, a polydnavirus replication protein, a poxvirus replication protein, an ascovirus replication protein, an iridovirus replication protein, a birnavirus replication protein, a togavirus replication protein, a replication protein of a flavivirus family member, a picornavirus replication protein, a tetravirus replication protein, or a nodavirus replication protein.

In one embodiment, the Linkage Polypeptide is a reovirus replication protein. In one mode of the embodiment, the reovirus is a plant reovirus. In another mode of the embodiment, the reovirus is an insect reovirus. In various embodiments, the Linkage Polypeptide is a rhabdovirus replication protein, such as, for example, a plant rhabdovirus or an insect rhabdovirus.

In another embodiment, the Linkage Polypeptide is a bunyavirus replication protein. In one mode of the embodiment, the bunyavirus is a plant bunyavirus. In another mode of the embodiment, the bunyavirus is an insect bunyavirus.

In yet another embodiment, the Linkage Polypeptide is a replication protein of a flavivirus family member (i.e., of the Flaviviridae family). In one mode of the embodiment, the flavivirus family member is a flavivirus. In another mode of the embodiment, the flavivirus family member is a pestivirus.

In still another embodiment, the Linkage Polypeptide is derived from Hepadnavirus polymerases. In various modes of the embodiment, the Linkage Polypeptide can be, for example, hepatitis B virus (HBV) reverse transcriptase (pol), derived from Hepadnavirus polymerase terminal protein (TP) domains, derived from Hepadnavirus-polymerase Reverse Transcriptase (RT) domains, polypeptide sequences derived from both Hepadnavirus polymerase TP and Hepadnavirus polymerase RT domains, polypeptide sequences derived from HBV polymerase terminal protein (TP) domains and/or HBV polymerase Reverse Transcriptase (RT) domains. In another embodiment, the Linkage Polypeptide includes polypeptide sequences derived from both HBV polymerase TP and HBV polymerase RT domains.

In certain specific embodiments of the invention, a Linkage Polypeptide can be a polypeptide that participates in the formation of a covalent bond with a nucleic acid, with the proviso that the Linkage Polypeptide is not naturally-associated with the Molecular Shaft (i.e., as found in nature). In a specific mode of embodiment, the Linkage polypeptide can be a polypeptide that participates in the formation of a covalent bond with a nucleic acid, with the proviso that the Linkage Polypeptide is not naturally-associated with the Molecular Shaft (i.e., as found in nature), when the Molecular Point is a label. In another specific embodiment, the Linkage Polypeptide can be a polypeptide that participates in the formation of a covalent bond with a nucleic acid, with the proviso that the Linkage Polypeptide is a fusion protein with the heterologous molecule (e.g., a protein or Molecular Point) not naturally-associated with the Linkage Polypeptide or the Molecular Shaft.

In an exemplary embodiment, the Linkage Polypeptide is Agrobacterium tumefaciens Ti plasmid-encoded VirD2. This Linkage Polypeptide is a member of a class of proteins that cut a DNA template in a sequence-specific manner, covalently bind to an end of the nicked DNA and facilitate the transfer of DNA into plant cells. Specifically, upon induction of the virulence program in A. tumefaciens, VirD2 and VirD1 excise a single-stranded DNA molecule (T-DNA) from the double-stranded tumor-inducing Ti plasmid. VirD2 recognizes two Recognition Sequence Motifs (each about 25 bases), called T-border sequences, within the T-DNA. In concert with VirD1, VirD2 interacts with the Recognition Sequence Motifs, cleaves the single-stranded DNA and forms a covalent linkage between a hydroxyl group on tyrosine 29 and the 5′ end of the cleaved single-stranded DNA. VirD2, in association with VirD1, also forms a covalent linkage with one strand of double-stranded DNA. In particular, VirD2 associates with a Recognition Sequence Motif on double-stranded DNA, nicks (i.e., cuts) one DNA strand and forms a covalent linkage with the 5′ end of the nicked strand. In vitro, these linkage reactions are reversible and require divalent cations (such as magnesium), but do not require ATP or other nucleoside triphosphate.

Another exemplary Linkage Polypeptide is E. coli RP4 plasmid-encoded TraI protein, a protein involved in bacterial conjugation. TraI is involved in the transfer of single-stranded DNA molecules from plasmid in one cell to another cell during conjugation. The molecular mechanism is similar to that of VirD2. A tyrosine residue on TraI forms a covalent linkage with the 5′ end of the nicked DNA. In one embodiment, the Linkage Polypeptide is a truncated or mutated form of TraI that possesses the capacity to covalently couple itself to single-stranded nucleic acids, but lacks the capacity to covalently couple itself to double-stranded (i.e., duplex) nucleic acids.

In other embodiments, the Linkage Polypeptide is a fusion protein comprising partial or full polypeptide sequences of an autocatalytic linkage protein and an accessory polypeptide factor that may participate in regulating or otherwise mediating the covalent coupling of Linkage Polypeptide sequences to nucleic acids. In one specific mode of this embodiment, the Linkage Polypeptide is a single polypeptide fusion containing partial or full polypeptide sequences of A. tumefaciens proteins virD1 and virD2.

Mutagenized and Evolved Linkage Polypeptides

In certain embodiments of the present invention, LPs can be mutagenized or ‘evolved’ proteins that can be created through the use of mutagenesis or directed evolution strategies, respectively. In these and other cases, ‘lead’ proteins for mutagenesis or directed evolution include those that can form transient covalent linkages with nucleic acid molecules, but cannot form stable covalent linkages (e.g., of sufficient stability to be useful for DART technology applications).

Mutants and evolved polypeptides can be derived from, for example, recombinases, integrases, topoisomerases, and restriction enzymes. Polypeptides that can be modified to form a covalent linkage with a nucleic acid encompassing a target nucleic acid sequence, can be, for example, mutants, variants, derivatives, and the like of such lead proteins. For example, the bacteriophage Cre recombinase forms a covalent 3′-phosphotyrosine linkage with DNA during the course of its catalysis of site-specific recombination between two 34-base pair loxP sites. Though the covalent bond is transient, it has been shown by site directed mutagenesis and high-resolution X-ray crystallographic studies to be essential for the progress of the reaction. (See, e.g., Guo et al., 1997, Nature 389:40-6). Similarly, in the phage lambda integrase system and the FLP recombinase system of the 2-μM plasmid of Saccharomyces cerevisiae, transient covalent bonds between conserved tyrosines of the recombinases and the 3′-phosphoryl groups at the sites of cleavage are formed (Zhu et al., 1995, J. Biol. Chem. 270:11646-11653). Some restriction enzymes and members of the eukaryotic type IB topoisomerase family (which include the mammalian nuclear topoisomerase I and vaccinia and other poxvirus topoisomerases) also catalyze DNA relaxation via similar mechanisms involving covalent DNA-(3′-phosphoamino)-protein intermediates. (See, e.g., Shuman, 1998, Biochim. Biophys. Acta. 1400:321-327; Jo and Topal, 1996, Biochemistry 35:10014-8; Colandene and Topal, 1998, Proc. Natl. Acad. Sci. USA 95:3531-6).

Molecules that form transient covalent linkages between proteins and nucleic acids can be used to generate mutants of LPs for DART technology applications. For example, in some embodiments of the present invention, these molecules can act as leads to be evolved or mutagenized into molecules that form stable covalent bonds with specific nucleic acids (e.g., mutants). Recombinase derivatives that can covalently couple themselves to preselected recombinase recognition sites can, for example, be selected in a screen in which a complex library of autonomously replicating expression vectors harboring in vitro mutagenized recombinase gene sequences are introduced into prokaryotic or eukaryotic cells containing corresponding recombinase recognition sites flanking a single counterselectable marker. After expression of the mutagenized recombinase genes, cells lacking active recombinase enzyme variants can be selected. These cells will likely harbor vectors coding for the expression of recombinase variants that form stable covalent linkages with target nucleic acid recognition sequences. These variants can be recovered and identified in secondary screens that directly or indirectly assess their ability to covalently couple themselves to nucleic acids. The skilled artisan will appreciate that similar screening strategies can be employed to identify integrase, topoisomerase, and restriction enzyme molecules with the desired covalent coupling properties.

In other embodiments of the present invention, Linkage Polypeptides can be generated by “domain swapping”. As will be apparent to the skilled artisan, “domain swapping” has proven a powerful tool for generating molecules with novel properties. For example, chimeric restriction enzymes that are hybrids between zinc finger DNA-binding domains and non-specific DNA cleavage domains of natural restriction enzymes (e.g., FOK I) can be synthesized and employed to cleave DNA at arbitrarily selected sequences in vitro and in vivo (see, e.g., Smith et al., 2000, Nucleic Acids Res. 28:3361-69). Chimeric LPs can be synthesized that harbor functional domains or motifs derived from other molecules with useful or desired properties.

In one embodiment, LPs possessing structures of the form A-B can be synthesized. These LPs can comprise of a single polypeptide harboring a DNA binding domain A (derived from protein A′) fused to domain B (derived from protein B′) that mediates the covalent linkage of B to nucleic acids. In a typical embodiment, the fusion protein (A-B) possesses properties lacking in either A or B alone, and provides for the generation of LPs that can covalently couple to DNA at selected sequences in vivo and in vitro.

In another embodiment, chimeric LP's can be prepared based on knowledge of the functional domain structure of Linkage Polypeptide and non-Linkage Polypeptide molecules. Below, an exemplary “parts list” of domains and motifs that can be assembled together to form a single novel LP molecule is provided. Though these exemplary parts originate from two sources, A. tumefaciens virD2 and like proteins, and integrases and recombinases, it will be apparent to the skilled artisan that other sequences of amino acids derived from other sources can also be employed for this purpose.

In one embodiment of the present invention, chimeric LPs including amino acid sequences present in A. tumefaciens virD2 and like proteins are employed. Specifically, two regions (Motif I and Motif III) of the virD2 family of proteins (that include TraI of the RP4 bacterial conjugation system) that harbor residues that participate in catalyzing covalent coupling reactions are employed, either jointly or separately, to construct chimeric LPs.

Motif I carries the tyrosine residue that covalently attaches these molecules in a trans-esterification reaction to the 5′ terminus of the cleaved DNA (see Table 1 below). Motif III contains a histidine essential for relaxase activity, and includes a conserved 14 residue segment, HxDxxx(P/u)HuHuuux (x any amino acid, u, hydrophobic residue), that directly participates in catalysis (see Table I below. Pansegrau et al., 1994, J. Biol. Chem. 269:2782-2789). In another embodiment of the present invention, Motif II residues that participate in DNA recognition can be employed to construct chimeric LPs (see Table 1 below). In a typical embodiment, natural or synthetic proteins harboring one or more polypeptide sequences with significant similarity to Motifs I, II, III can be employed as 20 Linkage Polypeptides for DART technology.

TABLE 1

Conserved Motifs Present in VirD2 Class Molecules

| | |
|---|---|
| PROTEIN | PROTEIN SEQUENCES |

| | | | |
|---|---|---|---|
| (Source) | Motif I | Motif II | Motif III |

| | | | |
|---|---|---|---|
| TraI (RP4) | FAELVKYITDEQGK | TYHLLV*SFRAGEK | QRSVSAVHHDTDNLHIHIAINKI |
| | (SEQ ID NO: 1) | (SEQ ID NO: 2) | (SEQ ID NO: 3) |
| TraI (R751) | FAGLANYITDAQSK | TYHLIV*SFRAGEQ | QRISAVHNDTDNLHIHIALNKI |
| | (SEQ ID NO: 4) | (SEQ ID NO: 5) | (SEQ ID NO: 6) |
| MobA(pTF-FC2) | VSRLTGYIREPERE | INHYVL*SWREGEQ | QAIYGLHADTDNLHLHLAINRV |
| | (SEQ ID NO: 7) | (SEQ ID NO: 8) | (SEQ ID NO: 9) |
| NikB (R64) | FSRLVDYATRLRNE | VFHYIV*SWQSHES | QYVSAVHTDTDNLHVHVAVNRV |
| | (SEQ ID NO: 10) | (SEQ ID NO: 11) | (SEQ ID NO: 12) |
| VirD2 (pTiC58) | IINQLEYLSRKGRL | TTHIIV*SFPAGTS | NYLTAFHIDRDHPHLHVVVNRR |
| | (SEQ ID NO: 13) | (SEQ ID NO: 14) | (SEQ ID NO: 15) |
| VirD2 (pRiA4b) | IINQLEYLSRKGRL | TTHIIV*SFPAGTS | NYLRAGHIDRDHPHLHVVVNRR |
| | (SEQ ID NO: 13) | (SEQ ID NO: 14) | (SEQ ID NO: 16) |
| VirD2 (pTiA6) | IINQLEYLSRKGRL | TTHIIV*SFPAGTD | NYLRAYHVDRDHPHLHVVVNRR |
| | (SEQ ID NO: 13) | (SEQ ID NO: 17) | (SEQ ID NO: 18) |
| R1x (pS194) | ASRAINYAEERAEE | QAHTVIQSFKPGE* | QVAVYTHTDKDHYHNHIIINSV |
| | (SEQ ID NO: 19) | (SEQ ID NO: 20) | (SEQ ID NO: 21) |
| R1x (pC221) | ASRAINYAEERAEE | QAHTVIQSFKPGE* | QVAVYTHTDKDHYHNHIVINSV |
| | (SEQ ID NO: 19) | (SEQ ID NO: 20) | (SEQ ID NO: 22) |
| R1x (pC223) | TSRAINYAEERAEE | EGHVVIQSFKPGE* | QVAVYTHNDTDHVHNHIVINSI |
| | (SEQ ID NO: 23) | (SEQ ID NO: 24) | (SEQ ID NO: 25) |

In certain embodiments of the invention, it will be desirable to generate chimeric LPs that include amino acid sequences present in recombinase and integrase family members. Specifically, these family members possess unique polypeptide sequences or functional domains that can be employed as ‘parts’ or ‘modules’ to be assembled into novel LP molecules. One aspect of this embodiment involves, for example, the E. coli phage lambda integrase protein (int). This protein belongs to a large Int family of site-specific recombinases. It is a heterobivalent DNA binding protein that makes use of a high energy covalent phosphotryosine intermediate to catalyze integrative and excisive recombination at specific chromosomal sites (Att sites). A 188 minimal catalytic fragment has been identified, and has three separate domains. Residues 1-64 recognize DNA; Residues 65-169 contribute to specific recognition of core-type sequences at sites of strand exchange and possibly to protein: protein interactions. Residues 170-356 carry out DNA cleavage and ligation. Tyrosine 342 is the active site nucleophile. (Radhakrishna et al., 1997, Proc. Natl. Acad. Sci. U.S.A. 94:6104-6109). In one embodiment of the present invention, naturally-occurring or chimeric molecules including assemblies of polypeptide sequences or functional protein domains similar to those present in E. coli phage lamda integrase can be used as lead molecules for subsequent directed evolution into proteins that form stable covalent bonds with nucleic acids, and can be useful for DART technology applications.

Non-Autocatalytic Linkage Polypeptides

In certain embodiments of the invention, it will be desirable to employ Linkage Polypeptides that lack autocatalytic linking activity. These Linkage Polypeptides can be divided into at least two classes.

One class includes Linkage Polypeptides that act as substrates for other trans-catalytic polypeptide activities. These trans-catalytic polypeptides catalyze the covalent linkage of non-autocatalytic Linkage Polypeptides to specific nucleic acid sequences, and can be derived from polypeptide sequences present in autocatalytic LPs. For example, a transcatalytic polypeptide that lacks the recipient amino acid to which the Molecular Shaft is linked can be generated. This transcatalyst can mediate the covalent coupling of a non-autocatalytic Linkage Polypeptide to, for example, PreMS or MS components, the Linkage Polypeptide harboring the recipient amino acid and requisite surrounding amino acids. Therefore, when the recipient Linkage Polypeptide and the transcatalytic polypeptide are placed in the presence of appropriate nucleic acid components, a DART can be formed. A system including both a transcatalytic virD2 derivative with inactivating mutations in Motif I and a non-autocatalytic Linkage Polypeptide variant of VirD2 with catalytically inactivating mutations in Motifs II and III provides an example of a system involving this class.

Another class of non-autocatalytic Linkage Polypeptides includes Linkage Polypeptides that require the presence of other polypeptides for their covalent linkage to nucleic acid targets. These non-autocatalytic Linkage Polypeptides typically cannot act alone. For example, in one embodiment of the present invention, systems derived from Hepadnavirus polymerases, in general, and hepatitis B virus (HBV) reverse transcriptase (pol), in particular, can be employed to make DARTs harboring non-autocatalytic Linkage Polypeptides. These reverse transcriptases are generally composed of four domains: (1) a Terminal Protein (TP) domain, which becomes covalently coupled to negative strand DNA by virtue of the protein-primed initiation of reverse transcription; (2) a Spacer domain, which is tolerant of mutations; (3) a Reverse Transcriptase (RT) domain; and (4) RNase H domain (for review, see Ganem and Varmus, 1987, Annu. Rev. Biochem. 56:651-693). In the HBV case, TP and RT are non-autocatalytic; when TP or RT is expressed alone in a recombinant baculovirus system, they cannot covalently couple themselves to HBV sequences. However, trans-complementation occurs when TP and RT are expressed together as independent polypeptides. Under these conditions, the TP polypeptide covalently links itself to HBV minus strand DNA sequences. Therefore, in one embodiment of the present invention, polypeptides derived from HBV TP and RT can be employed to synthesize non-autocatalytic LP-containing DARTs.

In another embodiment of the present invention, transcomplementing polypeptides derived from mutant variants of the bacteriophage P1 cre protein can be employed to synthesize non-autocatalytic LP-containing DARTs (see, e.g., Shaikh et al., 2000, J Biol. Chem. 275(39):30186-95).

The DARTs produced in these and similar systems possess a useful feature that can be absent in DARTs harboring autocatalytic LPs, namely, an inability to reversibly uncouple themselves from their corresponding Molecular Shaft components, even in the presence of postMS components. Consequently, these non-autocatalytic DARTs typically do not shuffle.

Linkage Polypeptide Fragments And Derivatives

In certain embodiments of the invention, a Linkage Polypeptide can be a fragment or derivative of a naturally-occurring protein. For example, a sequence that directs the subcellular localization of a Linkage Polypeptide can be deleted from or added to the Linkage Polypeptide so as redirect the Linkage Polypeptide to a different cellular compartment, or an extracellular compartment, from the one in which it naturally occurs.

In a typical embodiment, the Linkage Polypeptide does not oligomerize. In an exemplary mode of the embodiment, the Linkage Polypeptide is a truncation of VirD2 (e.g., the amino-terminal 196 amino acids of VirD2) that retains linkage activity but does not bind to the LP domain of MP-LP fusions or DARTs.

In another embodiment, a Linkage Polypeptide can comprise a NLS if a nuclear-localized Linkage Polypeptide is desired. In another embodiment, a Linkage Polypeptides can be genetically engineered to comprise a signal sequence. As used herein, a “signal sequence” refers to a sequence that can target the polypeptide to a desired location. Signal sequences can include, for example, a peptide of at least about 10 or 20 amino acid residues in length which occurs at the N-terminus of secretory and membrane-bound proteins and which contains at least about 70% hydrophobic amino acid residues such as alanine, leucine, isoleucine, phenylalanine, proline, tyrosine, tryptophan, or valine. The signal sequence is usually cleaved during processing of the mature protein.

It will also be appreciated by one of skill in the art that most of the Linkage Polypeptides described above exist as naturally-occurring variants. For example, the sequence of a viral capping protein will differ significantly among different strains of the same virus. All such variants which retain the ability to catalyze the formation of a covalent bond between the protein and a nucleic acid are within the scope of the present invention. In the case of DART formation through a trans-catalytic reaction, Linkage Polypeptide variants which retain the capacity to be covalent linked to a nucleic acid (by the action of a transacting protein) are encompassed by the invention.

The invention also encompasses Linkage Polypeptides that are chimeric or fusion proteins. As used herein, a “chimeric protein” or “fusion protein,” when referring to a Linkage Polypeptide, comprises all or part of a Linkage Polypeptide‘s native sequence operably linked to a heterologous polypeptide, while retaining the linking activity of the Linkage Polypeptide (i.e., catalysis of a covalent bond between the Linkage Polypeptide and a nucleic acid or the ability to be covalently linked to a nucleic acid by the action of a transcatalytic or transcomplementing protein). Within the fusion Linkage Polypeptide, the term “operably linked” is intended to indicate that the Linkage Polypeptide and the heterologous polypeptide are fused to each other. The heterologous polypeptide can be fused to the N-terminus or C-terminus of, or within, the Linkage Polypeptide.

One useful class of fusion proteins comprise Linkage Polypeptide linked to an affinity tag that may be used in purification, isolation, identification, or assay of expression of the Linkage Polypeptide. One useful fusion is a Glutathione-S-transferase (GST) fusion in which the Linkage Polypeptide is fused to the C-terminus of GST sequences. A Linkage Polypeptide can also be fused to the hemagglutinin (“HA”) tag or flag tag to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897). Alternatively, a Linkage Polypeptide can be fused to a polyhistidine, such as a hexahistidine, tag (allowing purification with a monoclonal antibody, e.g., 12CA5). Hexahistidine fusion proteins can be generated by cloning the nucleic acids encoding the Linkage Polypeptide into, for example, pET vectors (Novagen, Madison, Wis.). In another example, the fusion protein comprises thioredoxin to promote the solubility of a protein that would normally aggregate into inclusion bodies. Thioredoxin fusion proteins can be generated, for example, by cloning the Linkage Polypeptide into the pBAD/Thio vectors available from Invitrogen (Carlsbad, Calif.). Maltose E binding protein fusion proteins can be generated, for example, by cloning the Linkage Polypeptide into the pMAL vector (New England Biolabs, Beverly, Mass.). Protein A fusions can be generated, for example, by cloning the Linkage Polypeptide into the pRIT5 vector (Pharmacia, Piscataway, N.J.).

In certain embodiments of the present invention, a nuclear Linkage Polypeptide may be desired. For Linkage Polypeptides that do not have an endogenous nuclear localization signals, nuclear localization can be achieved by expressing the Linkage Polypeptide as a fusion protein comprising the Linkage Polypeptide with a heterologous nuclear localization sequence at its N-terminus, C-terminus, or inserted anywhere along the reading frame as long as it does not disrupt the linking activity of the Linkage Polypeptide. In a typical embodiment, the NLS is a simple NLS including a cluster of arginines and lysines, having a consensus of a hexapeptide often beginning or ending with a helix-breaking residue, such as proline, and containing three to five positively charged amino acids (reviewed by Boulikas, 1993, Crit Rev Eukaryot Gene Expr. 3(3):193-227). In a typical mode of the embodiment, the NLS is the NLS of the SV40 large T-antigen (PKKKRKV (SEQ ID NO: 26)); Kalderon et al., 1984, Nature 311 (5981):33-8). In another embodiment, the NLS is a ‘bipartite’ or ‘split’ NLS. Bipartite NLSs are defined as two separate sequences that cooperate to localize a single protein to the nucleus. Specifically, the bipartite NLS has two small clusters of positively charged amino acids (see, e.g., Dingwall and Laskey, 1991, Trends Biochem Sci. 16(12):478-81)—neither of which is functional as an NLS on its own—typically separated by 8-12 amino acids, including a PA dipeptide (Makkerh et al., 1996, Curr. Biol. August 6(8):1025-7). In yet other embodiments, the NLS used to direct the Linkage Polypeptide to the nucleus is an atypical NLS of the importin-independent class, such as those of hnRNP A1 (see, e.g., Siomi and Dreyfuss, 1995, J. Cekk Biol. 129:551-560) and hnRNP K (Michael et al., 1997, EMBO J 16:3587-98). Both these proteins shuttle in and out of the nucleus in a RNA polymerase II transcription-dependent manner, and their localization is mediated through the M9 and KNS (K Nuclear Shuttling) domains, respectively. M9 and KNS confer bi-directional transport across the nuclear envelope through separate pathways.

In certain embodiments of the present invention, a secreted Linkage Polypeptide may be desired. Production of a secreted Linkage Polypeptide can be achieved by expressing the Linkage Polypeptide as a fusion protein comprising the Linkage Polypeptide with a heterologous signal sequence at its N-terminus. For example, for secretion from a eukaryotic cell, the gp67 secretory sequence of the baculovirus envelope protein can be used as a signal sequence (Ausubel et al., supra). Other examples of eukaryotic signal sequences include the secretory sequences of melittin and human placental alkaline phosphatase (Stratagene; La Jolla, Calif.). When secretion of the Linkage Polypeptide from a prokaryotic cell is desired, the Linkage Polypeptide can be expressed, for example, as a fusion protein comprising the phoA secretory signal (Sambrook et al., supra) or the protein A secretory signal (Pharmacia Biotech; Piscataway, N.J.). Selection of an appropriate signal sequence can depend on the method of expressing the Linkage Polypeptide and can be determined by one of skill in the art.

The Linkage Polypeptide can further optionally include one or more subcellular localization sequences, secretion signals, sequences for directing DART or DART component insertion into biological or non-biological membranes, and the like.

The Linkage Polypeptide can further optionally include one or more protein or non-protein elements that contribute to the molecular stability of the Molecular Point, Linkage Polypeptide, Molecular Shaft, or combinations of these.

The Linkage Polypeptide can further optionally include one or more protein or non-protein sequences that mediate the oligomerization (e.g., dimerization, trimerization, multimerization) of Molecular Points, Linkage Polypeptides, Molecular Shafts, or combinations of these.

The Linkage Polypeptide can further optionally include one or more targeting sequences. These targeting sequences can direct the Molecular Point, Linkage Polypeptide, Molecular Shaft, or combinations of these, to the nucleus of a eukaryotic cell, the lumen or membrane of membrane bounded organelles (e.g., the endoplasmic reticulum, Golgi complex, endsome, lysosome, autophagic vacuole, chloroplast, mitochondria, plastid), or other subcellular regions of the cell.

The Linkage Polypeptide can further optionally include one or more targeting sequences., known to those skilled in the art, that direct the Molecular Point, Linkage Polypeptide, Molecular Shaft, or combinations of these, to be secreted outside the plasma membrane of the cell.

The Linkage Polypeptide can further optionally include one or more membrane anchoring amino acid sequences. These amino acid sequences can include, for example, GPI anchoring or myristylation sequence, and the like.

The Linkage Polypeptide can further optionally include one or more sequences, known to those skilled in the art, that direct that insertion of the polypeptide into a biological or non-biological membrane, and the like.

Synthesis of Linkage Polypeptides

A Linkage Polypeptide can be prepared by any suitable method. For example, a Linkage Polypeptide can be chemically synthesized by standard chemical synthesis techniques. Alternatively, a Linkage Polypeptide can be recombinantly expressed. Recombinant expression can be accomplished in vivo, for example by expression from an expression vector comprising the Linkage Polypeptide coding region operably linked to a promoter. Expression can be in a eukaryotic or prokaryotic cell. A Linkage Polypeptide can also be produced by an in vitro translation system, such as a reticulocyte lysate translation system.

A purified or isolated Linkage Polypeptide can be used in certain DART applications. A Linkage Polypeptide can be purified by using standard protein purification techniques. An “isolated” or “purified” Linkage Polypeptide (or biologically active portion thereof) is typically substantially free of cellular material or other contaminating proteins from the cell or tissue source from which the Linkage Polypeptide is derived, or substantially free of chemical precursors or other chemicals when chemically synthesized. The language “substantially free of cellular material” includes preparations of Linkage Polypeptide in which the Linkage Polypeptide is separated from cellular components of the cells from which it is isolated or recombinantly produced. Thus, Linkage Polypeptide that is substantially free of cellular material includes preparations of Linkage Polypeptide having less than about 30%, 20%, 10%, or 5% (by dry weight) of heterologous protein (also referred to herein as a “contaminating protein”). When the Linkage Polypeptide or biologically active portion thereof is recombinantly produced, it is also typically substantially free of culture medium, i.e., culture medium represents less than about 20%, 10%, or 5% of the volume of the Linkage Polypeptide preparation. When the Linkage Polypeptide is produced by chemical synthesis, it is typically substantially free of chemical precursors or other chemicals, i.e., it is separated from chemical precursors or other chemicals which are involved in the synthesis of the Linkage Polypeptide. Accordingly such preparations of the protein have less than about 30%, 20%, 10%, 5% (by dry weight) of chemical precursors or compounds other than the Linkage Polypeptide.

Nucleic acid sequences encoding Linkage Polypeptides that are chimeric or fusion proteins can be produced by standard recombinant DNA techniques. (See, e.g., Sambrook et al., supra; Ausubel et al., supra.) In another embodiment, the nucleic acid sequence can be synthesized by conventional techniques, including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a Linkage Polypeptide can be cloned into such an expression vector such that the fusion moiety is linked in-frame to the Linkage Polypeptide.

Molecular Shafts and Pre-Ms Molecules

The Molecular Shaft can be a nucleic acid, such as DNA, RNA or an RNA/DNA hybrid, and can be single-stranded, double-stranded or partially double-stranded. A single-stranded Molecular Shaft can be converted into a double-stranded Molecular Shaft, and vice versa. For example, a double-stranded Molecular Shaft can be formed from single-stranded shaft by synthesizing a complementary nucleic acid second strand or by annealing a complementary polynucleotide to the single-stranded Molecular Shaft. Conversely, a double-stranded Molecular Shaft can be converted into a single-stranded Molecular Shaft by, for example, exonuclease treatment and/or replication. Alternatively, a double-stranded Molecular Shaft can be formed as a consequence of the mechanism of DART synthesis. For example, rep protein-mediated DART formation generates a double-stranded Molecular Shaft.

A Molecular Shaft can be an mRNA, a cDNA or other nucleic acid of known or unknown function. In one embodiment, the Molecular Shaft corresponds to a known gene or a gene fragment. For example, the nucleotide sequence of the Molecular Shaft can correspond to a fragment encoding a domain of the protein encoded by the gene or a gene fragment corresponding to a specific exon of the gene or splice form of its encoded mRNA. The nucleotide sequence of the Molecular Shaft can differ from a known gene whilst encoding the same protein (due to degeneracy of the genetic code). Alternatively, the nucleotide sequence of Molecular Shaft can represent a mutant, variant or polymorph of a known gene. The nucleotide sequence of a Molecular Shaft can include a nucleotide sequence with about 50%, 55%, 60%, 65%, 75%, 85%, 95%, 98% or 99% sequence identity to the nucleotide sequence of a known gene or can encode a protein that includes an amino acid sequence that is at least about 50%, 55%, 60%, 65%, 75%, 85%, 95%, 98% or 99% identical to the amino acid sequence of a protein encoded by a known gene. The nucleotide sequence of a single-stranded Molecular Shaft or of the single-stranded portion of a partially double-stranded Molecular Shaft can be complementary to all or part of a coding sequence or 3′ or 5′ untranslated sequence of a known gene, and thus represent an antisense molecule.

A Molecular Shaft can be of any length, for example, about 10 to 10,000 bases or base pairs, or more, in length. In certain embodiments, the Molecular Shaft is about 20 to 7,500 bases or base pairs, about 50 to 5,000 bases or base pairs, about 100 to 1,000 bases or base pairs, about 250 to 500 bases or base pairs in length. In other embodiments, the Molecular Shaft is about 100 to 250 bases or base pairs, about 50 to 100 bases or base pairs, about 20 to 50 bases or base pairs, or about 10 to 20 bases or base pairs in length. In yet other embodiments, the Molecular Shaft is about 500 to 1,000 bases or base pairs, about 1,000 to 5,000 bases or base pairs, about 5,000 to 7,500 bases or base pairs or about 7,500 to 10,000 bases or base pairs in length.

A Molecular Shaft can be produced synthetically or recombinantly. Recombinant production of a Molecular Shaft can be accomplished in vivo (e.g., by expression in prokaryotic or eukaryotic cells by means of an expression cassette or vector) or in vitro (e.g., using in vitro transcription systems).

In certain embodiments, a Molecular Shaft can comprise or consist of RNA or DNA nucleotide analogs or modified nucleotides. For example, a Molecular Shaft can include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, or 2,6-diaminopurine. A Molecular Shaft can also include α-anomeric nucleic acid molecule (which forms specific double-stranded hybrids with complementary RNA in which, contrary to the usual β-units, the strands run parallel to each other (Gaultier et al., 1987, Nucleic Acids Res. 15:6625-6641)). The Molecular Shaft can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

One or more nucleotides of a Molecular Shaft can also be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see, e.g., Hyrup et al., 1996, Bioorganic & Medicinal Chemistry 4(1):5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under′ -conditions of low ionic strength. The synthesis of PNA oligomers can be performed, for example, using standard solid phase peptide synthesis protocols as described in Hyrup et al., supra; Perry-O'Keefe et al., 1996, Proc. Natl. Acad. Sci. USA 93:14670-675. PNAs can be modified, e.g., to enhance their (and therefore a Molecular Shaft's) stability or cellular uptake. In a specific embodiment, a pre-MS is a PNA-DNA chimera in which the portion recognized and/or cleaved by the Linkage Polypeptide is DNA while the remainder of the molecule is PNA. PNA-DNA chimeras can be linked using linkers of appropriate lengths selected in terms of base stacking, number of bonds between the nucleobases, and orientation, and the like. The synthesis of PNA-DNA chimeras can be performed as described in, for example, Hyrup (supra) and Finn et al., 1996, Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., 1989, Nucleic Acids Res. 17:5973-88). PNA monomers are then coupled in a stepwise manner to produce a chimeric molecule with a 5′ PNA segment and a 3′ DNA segment (Finn et al., 1996, Nucleic Acids Res. 24(17):3357-63). Alternatively, chimeric molecules can be synthesized with a 5′ DNA segment and a 3′ PNA segment (Peterser et al., 1975, Bioorganic Med. Chem. Lett. 5:1119-11124).

A Molecular Shaft can also comprise one or more nucleotides that have been modified to incorporate a detectable label (e.g., a radioisotope or a fluorescent compound).

A Molecular Shaft can further include one or more additional nucleic acid sequences, such as, for example, the following: a promoter to stimulate synthesis of nucleic acid complementary to a strand of the Molecular Shaft; one or more primer annealing sites for synthesis of a second strand from an annealed primer (e.g., by transcription, polymerase chain reaction (PCR), and the like); one or more restriction endonuclease sites to allow cleavage of the Molecular Shaft; a nucleic acid encoding a Linkage Polypeptide; a nucleic acid sequence(s) for hybridization of the Molecular Shaft to a complementary nucleic acid sequence for purification and/or localization to a discrete locus (e.g., on a DARTboard or other substrate); a nucleic acid sequence(s) encoding an epitope (e.g., an HA epitope, a polyhistidine tract, and the like); a nucleic acid sequence(s) encoding a linker polypeptide that allows greater flexibility between the Linkage Polypeptide and Molecular Point; a nucleic acid sequence(s) encoding a protease recognition site for cleavage of the Molecular Point and/or Linkage Polypeptide (e.g., a thrombin site), and/or a nucleic acid encoding a nuclear localization signal.

As will be apparent to one of skill in the art, a Molecular Shaft may further include a non-coding sequence that differs sufficiently in sequence from other nucleic acid sequences in a given population or reaction mixture that significant cross-hybridization does not occur. Such unique sequences can be employed as hybridization or identification tags. When multiple hybridization tags are utilized in a single reaction mixture, these tags also typically differ in sequence from one another such that each has a unique binding partner under the conditions employed. Unique non-coding regions can be incorporated into the nucleic acid component of the fusion for the specific purpose of being recognized by complementary nucleic acid sequences either immobilized on a surface or present in solution.

With the exceptions noted above, a preMS generally can have the features of a Molecular Shaft, and further comprises a Recognition Sequence Motif.

Recognition Sequence Motifs

A Recognition Sequence Motif is a nucleotide sequence present in a preMS and is recognized by a Linkage Polypeptide. The Recognition Sequence Motif optionally comprises a sequence for cleavage by a Linkage Polypeptide. If cleaved from a preMS, the Recognition Sequence Motif generally becomes part of the postMS. If a Recognition Sequence Motif is not cleaved by a Linkage Polypeptide, the Recognition Sequence Motif can remain as part of the Molecular Shaft when the Linkage Polypeptide is covalently attached to the preMS.

A Recognition Sequence Motif is generally at least 6 bases or base pairs in length, and may be up to hundreds of bases or base pairs. In certain instances, the Recognition Sequence Motif may be as few as 4 base pairs in length, for example when the Linkage Polypeptide is a modified restriction endonuclease that has a 4-base pair recognition site (e.g., AluI or HpaII).

The particular nucleotide sequence and length of a Recognition Sequence Motif can depend on the particular Linkage Polypeptide employed in DART formation and will be known to one of skill in the art. Exemplary Recognition Sequence Motifs are 5′ TATATCCTGT 3′ for VirD2 (SEQ ID NO: 27); 5′ CCTATCCTGC 3′ for Tra I (SEQ ID NO: 28); 5′ UUAAAACAG 3′ for poliovirus Vpg (SEQ ID NO. 29); and 5′ CATCATCATAATAT 3′ for adenovirus cap protein (SEQ ID NO: 30).

Molecular Points

As noted above, a Molecular Point is a non-nucleic acid molecule covalently linked to a Linkage Polypeptide, and is typically a polypeptide, including but not limited to an antibody.

A Molecular Point can further optionally be or include a variety of non-polypeptide components. These components may constitute or be derived from carbohydrates, oligosaccharides, polysaccharides, phospholipids, lipids, or other natural or non-natural products.

Like the Linkage Polypeptide, the Molecular Point can include one or more epitopes (e.g., a hemagglutinin epitope for the 12CA5 monoclonal antibody, a poly-histidine (e.g., 6-His tract, and the like), linker polypeptides, protease recognition sites (e.g., a thrombin site), protein or non-protein cleavable linkers, nuclear localization signals, and the like.

The Molecular Point can further optionally include one or more subcellular localization sequences, secretion signals, sequences for directing DART or DART component insertion into biological or non-biological membranes, and the like. The Molecular Point can further optionally include one or more protein or non-protein elements that contribute to the molecular stability of the Molecular Point, Linkage Polypeptide, Molecular Shaft, or combinations of these.

The Molecular Point can also optionally include one or more artificial polypeptides that are capable of presenting a peptide as a conformationally-restricted domain (e.g., a randomized peptide). The Molecular Point can further optionally include one or more protein or non-protein sequences that mediate the oligomerization (e.g., dimerization, trimerization, multimerization) of Molecular Points, Linkage Polypeptides, Molecular Shafts, or combinations of these.

The Molecular Point can further optionally include one or more targeting sequences. These targeting sequences can direct the Molecular Point, Linkage Polypeptide, Molecular Shaft, or combinations of these, to the nucleus of a eukaryotic cell, the lumen or membrane of membrane bounded organelles (e.g., the endoplasmic reticulum, Golgi complex, endsome, lysosome, autophagic vacuole, chloroplast, mitochondria, plastid), or other subcellular regions of the cell.

The Molecular Point can further optionally include one or more targeting sequences that direct the Molecular Point, Linkage Polypeptide, Molecular Shaft, or combinations of these, to be secreted outside the plasma membrane of the cell.

The Molecular Point can further optionally include one or more membrane anchoring amino acid sequences. These amino acid sequences can include, for example, GPI anchoring or myristylation sequence, and the like.

The Molecular Point can further optionally include one or more sequences that direct that insertion of the polypeptide into a biological or non-biological membrane, and the like (e.g., transmembrane domains).

The Molecular Point can further optionally include linear and cyclic polymers of polysaccharides, phospholipids, and peptides having either α-, β-, or Ω (omega)-amino acids, heteropolymers, small natural or synthetic molecules, or combinations of these.

As will become apparent below, the use of a DART can be determined, in part, by its Molecular Point. For example, DARTs comprising Green Fluorescent Protein (GFP) as a Molecular Point can be used as probes (infra). DARTs comprising RNAse H as a Molecular Point can be used to inhibit the expression of an oncogene and as cancer therapeutics (infra). A library of DARTs whose Molecular Points are mutants or variants of an enzyme can be used to identify counterparts of the enzyme with improved or altered activity (infra). DARTs with Molecular Points that are DNA binding domains or transcriptional activation domains (e.g., GAL4) can be used to identify novel transcriptional activation domains or DNA binding domains, respectively (infra).

Synthesis Of Darts And Dart Libraries

In another aspect, methods of preparing DARTs and libraries of DARTs are provided. DART libraries typically include at least a plurality of different DARTs (as the example in FIG. 1 illustrates). DART libraries can facilitate the assessment of a characteristic, function, and/or property of different Molecular Points, Linkage Polypeptides, Molecular Shafts and/or combinations of these.

A variety of DARTs and DART libraries can be prepared. A DART library can have at least a plurality of different Molecular Points linked to the same Linkage Polypeptide species. Such a DART library can be self referential, in which the Molecular Shafts encode the Molecular Points. Alternatively, the DART library can have at least a plurality of different Molecular Points covalently linked to the same Linkage Polypeptide species and Molecular Shaft species. A DART library can also have the same MP-LP pair species covalently linked to at least a plurality of different Molecular Shafts. In any of the various embodiments, the Molecular Shaft of DARTs can be RNA, DNA or an RNA/DNA hybrid, and can be single-stranded, partially double-stranded or double-stranded. DARTs and DART libraries can be synthesized and manipulated in vivo or in vitro.

DARTS can be generated when a Linkage Polypeptide becomes covalently linked to a preMS (or a portion thereof). For autocatalytic DART formation, the reaction conditions for the covalent linkage reaction are typically those conditions in which the Linkage Polypeptide and/or preMS is catalytically active. Such conditions will vary, as will be appreciated by the skilled artisan, depending on the Linkage Polypeptide. For example, a Linkage Polypeptide such as TraI and VirD2 can require the presence of a divalent cation, e.g., magnesium, for activity. The preMS molecule typically does not require any non-physiologic modifications to be covalently linked to the Linkage Polypeptide.

In Vivo Darts and Dart Libraries

For in vivo synthesis and manipulation, suitable hosts include prokaryotic cells (e.g., Escherichia coli or A. tumefaciens cells), eukaryotic cells (e.g., Saccharomyces cerevisiae, insect, or human cells), viral or phage particles, and the like.

DARTs and DART libraries can be prepared and manipulated in vivo. To preserve an informational relationship between the Molecular Points and the Molecular Shafts, DARTs can be prepared separately, and the resulting DARTs, or host cells containing the DARTs, pooled to form a library.

In one example, DARTs can be prepared using an expression construct. The expression construct typically includes a nucleic acid encoding a Linkage Polypeptide. The expression construct can also include at least one cloning site (e.g., a restriction site or polylinker) for insertion of a nucleic acid encoding a Molecular Point and/or Molecular Shaft. Such a restriction site(s) can be inserted (relative to the direction of transcription) at the 5′ end, the 3′ end, or within the sequence encoding the Linkage Polypeptide. The expression construct can also optionally include a nucleic acid sequence encoding a Recognition Sequence Motif. The Recognition Sequence Motif can be located 5′ or 3′ to the nucleic acid encoding the Linkage Polypeptide. If the DART is to encode the Linkage Polypeptide, the Recognition Sequence Motif is typically located 5′ to the sequence encoding the Linkage Polypeptide. In another embodiment, the nucleic acid encoding the Recognition Sequence Motif and a nucleic acid including the Molecular Shaft (or preMS molecule) can comprise a separate expression construct.

The expression construct typically includes a promoter. The promoter is selected according to the host organism in which the construct will be expressed (e.g., the GAL1-10 promoters for S. cerevisiae or the lac promoter for E. coli). The promoter is typically located 5′ to the sequence encoding the Linkage Polypeptide. Suitable promoters for expression in non-yeast eukaryotic systems include, but are not limited to, the SV40 early promoter region (Benoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42); plant expression vectors comprising the nopaline synthetase promoter region (Herrera-Estrella et al., 1984, Nature 303:209-213) or the cauliflower mosaic virus 35S RNA promoter (Gardner et al., 1981, Nucl. Acids Res. 9:2871), and the promoter of the photosynthetic enzyme ribulose biphosphate carboxylase (Herrera-Estrella et al., 1984, Nature 310:115-120); promoter elements from yeast or other fungi such as the Gal 4 promoter, the ADC (alcohol dehydrogenase) promoter, PGK (phosphoglyceroyl kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94); myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286), and gonadotropic releasing hormone gene control region which is active in the hypothalamus (Mason et al., 1986, Science 234:1372-1378). Suitable promoters for expression in yeast include, but are not limited to the GAL1 promoter (Johnston et al., 1984, Mol Cell. Biol. 8:1440-1448), which is repressed in the presence of glucose, the MET25 promoter (Kerjan et al., 1986, Nucl. Acids. Res. 14:7861-7871), which is induced by the absence of methionine in the growth medium, the CUP1 promoter, which is induced by copper (Mascorro-Gallardo et al., 1996, Gene 172:169-170), the CYCl promoter, which is repressed in the presence of glucose (Guarente and Ptashne, 1981, Proc. Natl. Acad. Sci. USA 78:2199-2203), and PHO5 which can be regulated by thiamine (Meyhack et al., 1982, EMBO J. 1:675-680). Promoters that are useful for controlling gene expression in prokaryotic systems include the araC promoter, which is inducible by arabinose (AraC), the TET system (Geissendorfer and Hillen, 1990, Appl. Microbiol. Biotechnol. 33:657-663), the pL promoter of phage λ temperature and the inducible lambda repressor CI857 (Pirrotta, 1975, Nature 254: 114-117; Petreako et al., 1989, Gene 78:85-91), the trp promoter and trp repressor system (Bennett et al., 1976, Proc. Natl. Acad. Sci USA 73:2351-55; Wame et al., 1986, Gene 46:103-112), the lacUV5 promoter (Gilbert and Maxam, 1973, Proc. Natl. Acad. Sci. USA 70:1559-63), 1 pp (Nokamura et al., 1982, J. Mol. Appl. Gen. 1:289-299), the T7 gene-10 promoter, phoA (alkaline phosphatase), recA (Horii et al., 1980, Proc Natl Acad Sci USA 77(1):313-7), and the tac promoter, a trp-lac fusion promoter, which is inducible by tryptophan (Amann et al., 1983, Gene 25:167-78).

The expression construct can be part of a vector. Such a vector may further include, for example, a positive selection marker to facilitate selection of transformants or transfectants and to ensure retention within the host organism. Suitable selection markers can include, for example, the LEU2, TRP1, or HIS3 genes for S. cerevisiae; the ampicillin, tetracycline or neomycin resistance genes of E. coli, or neomycin resistance for expression in mammalian cells. In some embodiments, the expression vector has at least two selection markers for selection in different organisms (e.g., in E. coli and in mammalian cells). Alternatively, a negative selection marker can be used to identify transformants or transfectants. Suitable negative selection markers include, but are not limited to, the bacterial codA gene encoding cytosine deaminase, which converts the non-toxic 5-fluorocytosine (5-FC) into the toxic 5-fluorouracil (5-FU), and bacterial cytochrome P450 mono-oxygenase gene, the product of which catalyses the dealkylation of a sulfonylurea compound, R7402, into a cytotoxic metabolite (both of which are discussed by Koprek et al., 1999, Plant J. 19(6):719-26)), a cassette comprising tetA from TN10, which confers resistance to tetracycline, osmotic sensitivity, and sensitivity to kanamycin and streptomycin, and rpsL from E. coli, which confers sensitivity to streptomycin, the result being as a synergistic enhancement of the osmotic, kanamycin, and streptomycin sensitivities (Stavropoulos and Strathdee, 2001, Genomics 72(1):99-104); and the Bacillus subtilis sac B gene (Lawes and Maloy, 1995, J. Bacteriol. 177: 1383-7), which confers sucrose sensitivity on gram negative bacterial strains. In addition to the foregoing negative and positive selectable marker systems, several vectors or markers have been developed that confer both negative and positive selection properties to a vector. Such positive/negative selection systems include, but are not limited to, the puDeltatk selection cassette, which comprises a bifunctional fusion protein between puromycin N-acetyltransferase (Puro) and a truncated version of herpes simplex virus type I thymidine kinase (DeltaTk) (Chen and Bradley, 2000, Genesis 28(1):31-5) and confers resistance to puromycin and sensitivity to 1-(-2-deoxy-2-fluoro-1-beta-D-arabino-furanosyl)-5-iodouracil (FIAU); and the TNFUS69 chimeric gene, which comprises a fusion of the HSV-tk and neomycin phosphotransferase II genes and confers resistance to neomycin and sensitivity to ganciclovir and (E)-5-(2-bromovinyl)-2′-deoxyuridine (Candotti et al., 2000, Cancer Gene Ther. 7(4):574-80). A set of five positive and negative selectable markers was created for use in mammalian cells, whose negative selectabilities are based on the Thymidine kinase (Tk) gene of Herpes Simplex virus (HSV) or the Cytidine deaminase (codA) gene of E. coli. The markers can be selected positively by their ability to induce either Hygromycin (Hyg), neomycin (neo), puromycin (PAC) or Blasticidin S (BlaS) resistance. With these markers, two complete sets of marker genes are available that induce independent negative selectable phenotypes. Exemplary methods in which plasmids harboring negative selection markers can be employed to introduce DART expression cassettes into host cells include monitoring the specific homologous recombination of appropriately constructed DART expression cassettes into the chromosome of target cells, host cell plasmids harboring appropriate homologous sequences, and the like.

The expression vector can also include an origin of replication, such as, for example, a pUC origin for replication in E. coli, an ARS1 or CEN sequence for replication in S. cerevisiae, and/or SV40 origin for replication in mammalian cells. Such a vector can also optionally include multiple expression constructs, for example, a first expression construct encoding a Molecular Point and Linkage Polypeptide, and a second expression construct encoding a preMS molecule.

The expression vector can also include retroviral packaging sequences (see, e.g., Rein, 1994, Arch Virol. Suppl. 9:513-22 for a review of retroviral packaging sequences).

In certain embodiments, the expression construct is an MP-LP expression construct. Such a construct typically includes a promoter for expression in the host organism, as discussed above. Downstream (i.e., 3′ relative to the direction of transcription of the promoter) is a coding sequence that encodes a Linkage Polypeptide. In some embodiments, the MP-LP expression vector further includes a Recognition Sequence Motif, which can be located either 5′ or 3′ to Linkage Polypeptide coding region. At least one cloning site (e.g., a restriction site or polylinker) can be included for insertion of a nucleic acid encoding a Molecular Point and/or Molecular Shaft. Such a cloning site(s) can be located at 5′ end, at the 3′ end, or within the Linkage Polypeptide coding region. The MP-LP expression construct can be part of a vector, which further includes a positive selection marker(s) and an origin of replication, as discussed above.

In vivo DNA Darts and Dart Libraries

A DNA DART library is typically prepared by isolating a plurality of different nucleic acids encoding different Molecular Points, inserting the nucleic acids into a cloning site, and the transforming or transfecting the expression vector into host cells. In certain embodiments, the expression vector can be a viral vector, including but not limited to a retroviral vector. The amount of expression vector to the number of cells transformed is typically adjusted so that each cell is successfully transformed, on average, with one expression vector. Thus, the MP-LP pair expressed in each host cell forms a DART with the corresponding Molecular Shaft.

A preMS molecule, and a Molecular Shaft, in the expression construct or vector can further include one or more additional nucleic acid sequences, such as, for example, the following: a promoter to stimulate synthesis of nucleic acid complementary to a strand of the preMS molecule or Molecular Shaft; one or more primer annealing sites for synthesis of a second strand from an annealed primer (e.g., by replication, transcription, polymerase chain reaction (PCR), and the like); one or more restriction endonuclease sites to allow cleavage of the preMS molecule or Molecular Shaft; a nucleic acid encoding a Linkage Polypeptide; a nucleic acid sequence(s) for hybridization of the preMS molecule or Molecular Shaft to a complementary nucleic acid sequence for purification and/or localization to a discrete locus (e.g., on a DARTboard or other substrate); a nucleic acid sequence(s) encoding an epitope (e.g., a hemagglutinin epitope for the 12CA5 monoclonal antibody, a poly-histidine (e.g., 6×His) tract, and the like); a nucleic acid sequence(s) encoding a linker polypeptide that allows greater flexibility between the Linkage Polypeptide and Molecular Point; a nucleic acid sequence(s) encoding a protease recognition site for cleavage of the Molecular Point and/or Linkage Polypeptide (e.g. a thrombin site), and/or a nucleic acid encoding a nuclear localization signal.

In vivo DNA Darts and DART Libraries In Bacteria

DNA DARTs and DART libraries can be prepared using an expression construct or vector, such as an MP-LP expression construct or vector. The construct or vector typically includes a promoter suitable for expression in the host organism (e.g., the lac promoter for E. coli expression), and, 3′ to the promoter, a nucleic acid encoding a Linkage Polypeptide that is active in the chosen bacterial host (e.g., TraI in E. coli). At least one cloning site (e.g., a restriction site or polylinker) for in frame insertion of a nucleic acid encoding a Molecular Point is typically included at the 5′ end of, the 3′ end of, or within, the sequence encoding the Linkage Polypeptide. The expression construct can also optionally include a nucleic acid sequence encoding a Recognition Sequence Motif, which can be positioned 5′ or 3′ of the nucleic acid sequence encoding the Linkage Polypeptide. In some embodiments, the MP-LP vector includes a positive selection marker (e.g., beta-lactamase for E. coli) to ensure the retention of the expression vector within a transformed host cell. The MP-LP expression vector can also include nucleic acid sequences for the replication of the expression vector in the host cell (e.g., pUCori for E. coli).

The preMS molecule can be any suitable DNA template, including but not limited to a single-stranded DNA (e.g., synthesized using bacteriophage M13) or double-stranded DNA. The MP-LP pair can be covalently linked to a double-stranded nucleic acid (e.g., two hybridized complementary nucleic acids) under similar conditions in the presence of auxiliary factors (e.g. VirD1 in the case of the VirD2) required for Linkage Polypeptide cleavage of a double-stranded template. A Recognition Sequence Motif is typically located 5′ of coding sequences for the MP-LP pair. For example, to link an MP-LP pair to sequences encoding the MP, the LP Recognition Sequence Motif is positioned 5′ relative to the MP coding region. In another example, to link a MP-LP pair to a preMS molecule having both MP and LP coding sequences, the Recognition Sequence Motif is positioned 5′ to both the LP and MP coding sequences.

In vivo DNA Darts and DART Libraries in Eukaryotes

The methods for DNA DART synthesis in bacteria can be adapted for use of DNA DART synthesis in eukaryotes, as will be appreciated by the skilled artisan, and can include the use of a expression cassettes or vectors. In certain embodiments, the following modifications can be made. The MP-LP expression vector typically includes a selection marker that is appropriate for the host cell (e.g., geneticin resistance for mammalian cells or LEU2 for S. cerevisiae). In some embodiments, the MP-LP expression vector includes positive selection markers suitable for use in bacteria and for use in eukaryotes. The MP-LP expression vector can also encode a Linkage Polypeptide that is active in the host organism (e.g., the adenovirus cap protein in human cells). As will be appreciated by the skilled artisan, the codon usage of the nucleic acid encoding the Linkage Polypeptide optionally can be optimized for expression in the desired host organism. The DARTs can also include a nuclear localization signal. For example, if the preMS molecule is expected to reside in the cell nucleus, then the MP-LP pair can include a nuclear localization signal (NLS) that directs the MP-LP pair to the nucleus (e.g., a nuclear localization signal in the Linkage Polypeptide). The MP-LP expression vector can optionally include sequences for the replication of the vector within the host cells (e.g., the ARS1 sequence for S. cerevisiae or the SV40 origin for mammalian cells).

In vivo RNA DARTs and DART Libraries

RNA DARTs and libraries can also be prepared in vivo or in vitro. RNA DART libraries have Molecular Shafts of RNA or RNA/DNA hybrids. An informational relationship can exist between the Molecular Point and the Molecular Shaft of an RNA DART. RNA DARTs are typically prepared in vivo by allowing a newly translated Linkage Polypeptide (or an MP-LP pair) to interact with a preMS molecule (e.g., a transcript or messenger RNA). The MP-LP pair can also be synthesized in vivo. The Linkage Polypeptide, or MP-LP pair, and the preMS molecule can be brought into close physical proximity with each other by the ribosome. Thus, RNA DARTs can be synthesized in host cells using this methodology because each mRNA can be covalently linked to the MP-LP pair that it encodes. RNA DART synthesis methodology can be employed to synthesize DARTs in prokaryotic cells and/or eukaryotic cells. The Linkage Polypeptide is selected according to the synthesis conditions.

RNA DART expression constructs and vectors can be used in the synthesis of RNA DARTs. An RNA DART expression construct can typically have the following features: a promoter, a nucleic acid encoding a Linkage Polypeptide, and cloning site for nucleic acids encoding the Molecular Point and/or Molecular Shaft. The cloning site(s) can be located 5′, 3′ and/or within the Linkage Polypeptide coding region. In additional embodiments, the expression construct contains a nucleic acid encoding a Molecular Point and/or Molecular Shaft. A Recognition Sequence Motif can be located 5′ or 3′ to the Linkage Polypeptide coding region. In certain embodiments, the Recognition Sequence Motif is located 5′ of nucleic acid sequences to which the LP domain of MP-LP pair will be covalently linked. For example, to covalently link a MP-LP pair to a preMS molecule encoding the Molecular Point, the Recognition Sequence Motif can be placed 5′ of the Molecular Point coding region. Alternatively, to make RNA DARTs having a Molecular Shaft that encodes both the Linkage Polypeptide and the Molecular Point, the Recognition Sequence Motif can be placed 5′ to both the LP and MP coding regions.

An RNA DART expression vector typically includes an expression construct, a positive selection marker and sequences for replication of the vector within the host organism (e.g., pUCori for E. coli). In one embodiment, a ribosome pause site is optionally positioned 3′ of the MP and LP coding sequences. This pause site can be a series of rare codons, a self-complementary sequence that anneals to form a hairpin structure, or another element that slows or temporarily delays translation or release of the RNA from the ribosome.

In an exemplary embodiment, an RNA DART is formed as follows: RNA encoding the MP-LP pair is transcribed (a preMS molecule). Following initiation of RNA transcription, translation begins, which results in production of the MP-LP pair. The Linkage Polypeptide of the MP-LP pair recognizes the Recognition Sequence Motif within a preMS molecule. The Linkage Polypeptide and the Recognition Sequence Motif then interact to form a RNA DART. If the transcribed RNA contains a ribosomal pause site, translation on the ribosome can be stalled, which can allow the LP to interact with the Recognition Sequence Motif while the RNA ′ (preMS molecule) is bound to the ribosome.

In Vivo RNA Libraries In Bacteria

The RNA DART synthesis methods described herein can be employed to synthesize RNA DARTs in vivo in bacteria. An expression vector, such as an MP-LP expression vector, can transformed or transfected into suitable host cells (e.g., E. coli). The expression vector can include a promoter suitable for function in the host organism. Downstream (3′) of the promoter is the coding region for the Linkage Polypeptide. The Linkage Polypeptide is selected to be active in the eukaryotic host cells. Cloning sites for the Molecular Point and/or Molecular Shaft are located 5,′3′ and/or within the coding region for the Linkage Polypeptide. A Recognition Sequence Motif is typically located 5′ of nucleic acids encoding the Molecular Point. If the resulting RNA DART will encode an MP-LP pair, the Recognition Sequence Motif is placed 5′ of the MP and LP coding regions. The expression vector can also include a positive selection marker and/or a nucleic acid sequence(s) for replication of the vector within the host organism (e.g., pUCori for E. coli), as desired. The Linkage Polypeptide is typically selected to be active in the desired host.

In Vivo RNA Libraries in Eukaryotes

The methods and systems for RNA DART synthesis in bacteria can be adapted to allow RNA DART synthesis in eukaryotes, and can include the use of expression cassettes and vectors. In certain embodiments, the following modifications can be used: The MP-LP expression vector can optionally include sequences for its replication within the eukaryotic host organism (e.g., ARS1 for S. cerevisiae or the SV40 origin for expression in mammalian host cells). The expression vector can encode an mRNA molecule. The Linkage Polypeptide is selected to be active in the eukaryotic host cells (e.g., poliovirus Vpg protein in a mammalian cell).

In vitro DARTs And DART Libraries

DARTs and DART libraries can be synthesized in vitro, as described below.

In vitro DNA DARTs And DART Libraries

DNA DARTs can be prepared by a variety of in vitro procedures. To preserve an informational relationship between the Molecular Points and the Molecular Shafts, each DART can be prepared separately, and the resulting DARTs pooled to form a library. In vitro DARTs and DART libraries can optionally include any of the additional features described herein, such as, for example, nucleic primer annealing sites, promoters, positive selection markers, origins of replication, epitopes, and the like.

In one embodiment, a DNA DART library is prepared by combining a plurality of MP-LP pairs with a plurality of preMS molecules under the appropriate reaction conditions and allowing the DARTs to self assemble. The conditions for the covalent linkage reaction are selected according to those required for Linkage Polypeptide and/or preMS molecule catalytic activity. In another embodiment, an in vivo system, such as any those described above, can be used to prepare DARTs in vitro. Such in vitro DARTs are typically prepared using a cell lysate from a host cell, but can also be prepared from purified or semi-purified components. For example, an MP-LP expression vector can be used that encodes a Linkage Polypeptide that is active under the chosen in vitro conditions (e.g., conditions suitable for TraI activity). The MP-LP pair can optionally be at least partially purified prior to addition to the covalent linkage reaction. Alternatively, the MP-LP pair can be present in the host cell extract (e.g., an extract from a host cell expressing the MP-LP pair). In such an alternative, the MP-LP pair is typically not expressed in cells having nucleic acids encoding the Recognition Sequence Motif for the Linkage Polypeptide to limit DART formation in vivo.

The preMS molecules can be added to the in vitro reaction. For example, the preMS molecules can be synthesized in vitro by Polymerase Chain Reaction, or similar synthetic processes. Alternatively, the preMS molecules can be provided as an extract(s) from host cells expressing the preMS molecules. The preMS molecules can optionally be partially purified prior to addition to the covalent linkage reaction.

In another embodiment, DARTs can be synthesized in vitro using coupled transcription and translation systems. Typically, preMS molecules are incubated with transcription/translation extracts under conditions known to the skilled artisan (Promega technical manual No. 235 and references therein). The MP-LP translation product of the preMS molecule typically contacts the preMS in a cis-acting fashion and covalently links MP-LP and preMS to form a self-referential DART.

In yet another embodiment, DARTs can be synthesized on an affinity substrate, such as a DNA microarray. Specifically, Linkage Polypeptides can be linked to preMS molecules immobilized on a solid surface. The solid surface can be, for example, a glass slide, silicon wafer, a well of a microtiter plate, agarose beads, a nitrocellulose membrane, a nylon membrane, a PVDF membrane, or any other solid or semi-solid surface. In this example, preMS molecules can act as capture reagents for Linkage Polypeptides (LP) and any linked Molecular Point (MP). In one method, a robot, mechanical, manual, or other deposition device can be used to contact the MP-LP with Capture Molecular Targets deposited on a substrate. In another method, MP-LP fusion pairs can be incubated with Capture Molecular Targets deposited on a substrate. The incubation is carried out under conditions that allow the MP-LP pair and the preMS to become linked. Each MP-LP pair can target and link to Capture Molecular Targets comprising its cognate preMS sequences. This method of DART formation is exemplified infra.

In vitro RNA DARTs and DART Libraries

RNA DARTs and libraries can also be prepared in vitro. RNA DART libraries have Molecular Shafts of RNA or RNA/DNA hybrids. An informational relationship can exist between the Molecular Point and the Molecular Shaft of an RNA DART. RNA DART synthesis can be performed in vitro using cell extracts, partially purified or purified components (e.g., MP-LP pairs and preMS molecules). As discussed above, in any of the various embodiments of RNA DARTs, an informational relationship can exist between the Molecular Point and the Molecular Shafts of RNA DARTs.

RNA DARTs can also prepared by a variety of methods known to the skilled artisan. If an information relationship is desired between the Molecular Point and the Molecular Shaft, the covalent linkage reaction(s) can be performed individually, or in small pools, and the resulting RNA DARTs combined to form a library, as desired. In one embodiment, purified or partially purified MP-LP pairs are combined with preMS molecules (purified or partially purified) in vitro under the appropriate reaction conditions. Alternatively, extracts from host cells can be used. For example, any of the MP-LP expression systems described herein can be used to prepare RNA DARTs in vitro. In some embodiments, the following modifications to that system can be included: The RNA encoding the MP-LP pair (i.e., the preMS molecule) can be synthesized by a variety of means, as will be appreciated by those skilled in the art. Such means include, but are not limited to, in vitro transcription (e.g., transcription of pooled RNA DART expression constructs or vectors). The Linkage Polypeptide is typically selected such that it is active under the chosen in vitro conditions (e.g. polio virus Vg protein in mammalian cell cytoplasmic extracts). The covalent linkage reaction can optionally be performed in vitro in a coupled transcription-translation system (e.g., a coupled rabbit reticulocyte extracts, wheat germ or E. coli transcription/translation system). In such coupled transcription-translation systems, the MP-LP pair need not be separately synthesized or purified.

Complex libraries of DART molecules can be constructed using methods that are known to those skilled in the arts. For example, in one embodiment of the present invention, Molecular Point components of DART populations can be derived from the in vitro transcription and translation, or in vivo expression, of nucleic acids harboring biased or unbiased cDNA or genomic libraries of various sizes. These libraries can, for example, be generated from viruses, prokaryotic cells, eukaryotic cells (including human cells), or combinations of these. Similarly, nucleic acids harboring genomic or cDNA libraries of known or unknown genes, or cell-specific, tissue-specific, and/or organism-specific cDNAs of various sizes may be employed. In another embodiment, nucleic acids harboring synthetic libraries containing sequences derived from randomized or biased oligonucleic acid synthesis can be employed. In yet another embodiment, libraries of various sizes encoding specific classes of molecules, including, for example, kinases, oncoproteins, transcription factors, phosphatases, membrane proteins, membrane receptors, steroid receptors, and the like, can be employed for library production. In yet another embodiment, nucleic acids harboring libraries of genes strictly not present, or strictly including, those found in the host organism in which the library is expressed can be employed. Complex populations of DART molecules can be screened using a wide variety of nucleic acid and/or protein screening methods known to those skilled in the arts, or other methods described herein.

In yet other embodiments, RNA DARTs can be synthesized using coupled transcription and translation systems or on an affinity substrate, as described, for example, for DNA DARTS (supra). One of skill in the art can readily modify these methods to produce RNA rather than DNA DARTs.

DART Ligation

In another aspect according to the present invention, methods for DART ligation are provided. DART ligation is the joining of a Molecular Shaft to another nucleic acid, such as a preMS molecule, postMS molecule or the Molecular Shaft of another DART. Typically, the Linkage Polypeptide of a DART catalyzes the ligation reaction that joins the Molecular Shaft (i.e., the nucleic acid linked to the LP) to the other nucleic acid, a preMS Molecule. The preMS molecule has a Recognition Sequence Motif that is recognized by the Linkage Polypeptide. For example, VirD2 and TraI can ligate the 5′ terminus of the nucleic acid molecule to which it is covalently linked (referred to as Molecular Shaft 1or MS₁) to the 3′ terminus of another nucleic acid molecule (MS₂′) containing the LP Recognition Sequence Motif at its 3′ terminus (e.g., a postMS molecule) in the presence of magnesium, or another suitable divalent cation. The resulting DART ligation product (e.g., MS₂′-RS-MS₁) is a nucleic acid with a Recognition Sequence Motif between the other nucleic acid molecule (e.g., MS₂′) and the Molecular Shaft (e.g., MS₁).

DART ligation can be employed, for example, to clone another nucleic acid by attaching it to a Molecular Shaft, such as MS 1, or to attach a nucleic acid molecule (e.g., the Molecular Shaft) to a substrate, column or another nucleic acid molecule containing a Recognition Sequence Motif. The released LP (or MP-LP) can then be coupled to a new nucleic acid molecule (i.e., a preMS molecule) containing an appropriate Recognition Sequence Motif.

Polyvalent DARTs

In another aspect, Polyvalent DARTs are provided. A Polyvalent DART has two or more Linkage Polypeptides covalently linked to a Molecular Point. Each Linkage 25 Polypeptide can covalently link to a separate nucleic acid containing an appropriate Recognition Sequence Motif (e.g., preMS molecule). For example, an MP-LP₁-LP₂ Polyvalent DART can be linked via LP₁, to preMS₁, which contains Recognition Sequence Motif 1 (RS I) recognized by LP₁. The second Linkage Polypeptide, LP₂, can be covalently linked to preMS₂, which contains Recognition Sequence Motif 2 (RS₂) (recognized by LP₂). 30 The Linkage Polypeptides can be linked to each other and/or to the Molecular Point.

In one embodiment, to ensure coupling specificity, each LP (e.g., LP₁ and LP₂) is different, such that each LP interacts with a different Recognition Sequence Motif. For example, LP₁ (e.g., TraI) can react specifically with RS₁, in preMS₁, but not RS₂, to form a covalent linkage with MS₁. Conversely, LP₂ (e.g., virD2) recognizes RS₂ in preMS₂ to covalently link itself to MS₂, but does not recognize RS₁ in preMS₁. The formation of Polyvalent DARTS can be performed sequentially or simultaneously using methods described herein. Alternatively, the LP's can recognize the same RS.

DART Shuffling

In another aspect, methods for DART shuffling, and for limiting DART shuffling, are provided. DART shuffling is the exchange of Molecular Shafts between DARTs, or between DARTs and postMS, preMS or other nucleic acid molecules. In some embodiments, DART shuffling is random, resulting in the linking and unlinking of different MP-LP pairs and postMS molecules, and therefore creating a disordered collection of DARTs whose Molecular Points and Molecular Shafts no longer correspond to one another or retain a useful informational relationship. In another embodiment, DART shuffling can be used to generate and/or modify non-DART molecules.

DART shuffling results from the reversibility of the interaction of the Linkage Polypeptide and a nucleic acid associated with a Recognition Sequence Motif. The covalent linkage reaction between the preMS molecule and the MP-LP produces a DART (MP-LP-MS complex) and a postMS molecule. For many Linkage Polypeptides (e.g., TraI and VirD2), the covalent linkage reaction is readily reversible under the appropriate reaction conditions (e.g., no external energy source is required). Notably, the two products of the covalent linkage reaction, a DART and postMS molecule, can react with one another to form the starting reactants for the covalent linkage reaction, an MP-LP pair and a preMS molecule.

This reversibility of the covalent linkage reaction can be utilized to control the covalent linkage reaction, and thereby control DART shuffling. In some embodiments, if DART shuffling is desired (e.g., DARTex, as discussed below), higher concentrations of postMS molecules can be added to promote shuffling. In other embodiments, DART shuffling can compromise the informational content of the DARTs. In such embodiments, DART shuffling can be limited by controlling Linkage Polypeptide activity and/or the presence of postMS molecule in the reaction. Generally, if the Linkage Polypeptide is active and postMS molecules are present when different species of LP-containing DARTs are mixed, then DART shuffling will likely occur.

DART shuffling can be prevented in a number of ways. For example, the reactivity of postMS molecules can be inhibited. This can be achieved by several methods including, but not limited to, removal or degradation of postMS molecules using chemical, enzymatic or physical (e.g., dialysis or other size fractionation methods) methods; separation of postMS molecules from DARTs as a consequence of DART purification (see infra); and the addition of a nucleic acid complementary to postMS molecules that forms a duplex with postMS molecules and prevents their reactivity with the Linkage Polypeptide. Alternatively, the covalent linkage reaction can be inhibited by addition of chemicals, polypeptides, or enzymes that inhibit Linkage Polypeptide activity (e.g., antibodies that bind LP; chelators such as EDTA that sequester or remove a divalent cation required for LP activity), the removal or absence of a factor required for LP activity (e.g., magnesium or another divalent cation), the separation of postMS molecules from DARTs as a consequence of DART purification, and the like. For DARTs synthesized using involving trans-catalytic or trans-complementary polypeptides, shuffling can be prevented, for example, by removing these molecules.

DART Purification/Enrichment

In some aspects, DART purification or enrichment is advantageous. For example, increasing the concentration of DARTs can enhance the sensitivity of some DART detection schemes, as discussed herein. High concentrations of DART molecules can facilitate some applications, such as those in which particular DART species will interact with other molecules. Similarly, DART purification or enrichment, can be advantageous to separate DARTs, or DART components, from conditions in which DART physical or functional integrity might be compromised. It can also be advantageous to separate DARTs from non-DART molecules that can interfere with DART activities (e.g., removing molecules that stimulate DART shuffling), such as, for example, free Linkage Polypeptide, free transcatalytic or transcomplementing polypeptides, free MP-LP pairs, free preMS molecules, and/or free postMS molecules). DART purification or enrichment can also be useful for DARTboards, DARTex, DARTdance applications, and the like.

Various strategies can be employed to obtain enriched DARTs, components and related or desired interacting molecules, including, but not limited to, affinity purification, direct preparation, analytical chemical strategies, and the like. Such strategies can provide fractions enriched in DART molecules, components, and associated or desired interacting molecules.

These strategies can also provide fractions enriched in multi-molecular DART-containing complexes. These complexes can include but are not limited to those harboring a DART molecule covalently linked or non-covalently bound to one or more DARTs or non-DART molecules. According to one embodiment, the invention provides a methodology for chemically trapping-low affinity interactions between DARTs and non-DART molecules. For example, commercially available cross-linkers, known to those skilled in the art, can be employed to covalently couple, in vitro or in vivo, non-DART molecules to their DART counterparts. In addition, cross-linkers can be designed to be specific for particular DART:non-DART complexes while having relatively no specificity or affinity for either component alone. Accordingly, one skilled in the arts will appreciate that it is possible to covalently trap the non-DART components bound to DART molecules.

DARTs can be purified or isolated from cells, tissues and/or complex solutions in which they are prepared, manipulated or stored by, for example, direct protein and/or nucleic acid chromatographic and analytical chemical methods. Such methods include, but are not limited to, capillary gel electrophoresis, reverse or fluid phase (e.g., anion exchange, cation exchange, hydroxyapatite chromatography, and the like) chromatography, liquid chromatography or high performance liquid chromatography (HPLC). The presence of DARTs in fractions obtained from these methods can be ascertained using a variety of methods, as will be appreciated by the skilled artisan.

Affinity purification strategies can also be used. Such strategies can exploit the reversible, specific, covalent and/or non-covalent interactions between one or more Capture Molecular Targets (CMTs) and a molecule that can specifically bind to the Capture Molecular Target, such as a DART (generally referred to as an Affinity Target or AT). Affinity purification strategies can include the use of Capture Molecular Targets (CMTs) such as nucleic acids, polypeptides, antibodies and/or non-protein molecules, to isolate DARTs, DART components and/or DART-related molecules that bind to the Capture Molecular Target.

Generally, affinity purification will include steps of substrate preparation, blocking, binding, washing, and elution, although one or more of these steps can be omitted in some embodiments. For example, substrate preparation can be omitted if the binding interaction is performed in solution. The following discussion exemplifies various procedures by which these steps can be performed.

Substrate Preparation: A substrate, such as a Purification Substrate, can be prepared. Substrates can be prepared by, for example, application of Capture Molecular Targets to a substrate. For example, nucleic acids can be the Capture Molecular Targets that are attached to a substrate. These Capture Molecular Targets can range in length from as few as ten bases to hundreds of base pairs up to a million bases or more. The nucleic acids can be prepared from any suitable source, such as synthetic polynucleotides, or from natural sources, such as from mammals, animals, insects, viruses, parasites, plants, or other organism, as well as in vitro culture constituents of these. In some embodiments, the nucleic acids are DNA, RNA, or RNA/DNA hybrids.

The Capture Molecular Targets can be attached to the substrate using techniques known to the skilled artisan. In some aspects of the invention, the substrate can optionally further include a backing. For example, a backing can be a glass slide or rigid polymer sheeting. The substrate can be applied to a suitable backing by spraying or coating uncured material onto the backing, or by applying a preformed substrate, such as a membrane, to the backing. The backing and substrate can be obtained as a preformed unit from commercial source (e.g., a plastic-backed nitrocellulose film available from Schleicher and Schuell Corporation) or it can be prepared as needed.

The Capture Molecular Targets can be laid out on the substrate in any suitable pattern, such as, for example, a square or rectangular grid. In one embodiment, the array is formed with nucleic acids at known, addressable regions of an array. For example, a 96-cell array can be formed with dimensions between about 1.2 and about 24.4 cm in width and between about 0.8 and about 40.0 cm in length with the cells in the array having width and length dimension of about 1/12 and about ⅛ of the array width and length dimensions.

The substrate can optionally be partitioned by forming water-impermeable grid lines. Such grid lines can infiltrate a film down to the substrate or backing, and can extend above the surface of the film to form a well. For example, the grid lines can be formed by laying down an uncured or otherwise flowable resin or elastomer solution in a grid pattern, allowing the material to infiltrate the porous film down to the backing, then curing or otherwise hardening the grid lines.

One exemplary material for the grid is a flowable silicone. The grid material can be extruded through a narrow syringe (e.g., 22 gauge) using air pressure or mechanical pressure. The syringe is moved relative to the solid support to form grid elements in a pattern. The extruded silicone wicks into the pores of the solid support and cures to form a shallow waterproof grid separating the regions of the solid support. In alternative embodiments, the grid can be a wax-based material or a thermoset material, such as epoxy.

The grid material can also be a UV-curing polymer that is exposed to UV light after being printed onto the solid support. The grid can also be applied to the solid support using printing techniques such as silk-screen printing. The grid can also be a heat-seal stamping of the porous solid support which seals its pores and forms a water-impervious grid. The grid can also be a shallow grid that is laminated or otherwise adhered to the solid support.

Each well can contain a single type of Capture Molecular Target (i.e., a single species of nucleic acid) or it can contain an array (including a microarray) of different species of Capture Molecular Targets. In one embodiment, the Capture Molecular Targets in each well are identical. In another embodiment, the array can be formed by depositing a first selected Capture Molecular Target at a first selected position in one or more cells, then depositing a second different Capture Molecular Target at a different position in one or more cells, and so on until a complete array is formed in one or more cells on the substrate.

The Capture Molecular Targets can be attached to the substrate using, for example, a coating of a material for binding or immobilizing the Capture Molecular Targets. Such a material can include, for example, a polycationic polymer, such as a cationic polypeptide (e.g., poly-1-lysine and/or polyarginine). The substrate is coated by placing a substantially uniform thickness of the coating material (e.g., poly-1-lysine) on the surface of the substrate and then drying the film to form a dried coating. The amount of coating material is sufficient to form at least a monolayer of material on the surface at the location(s) where the Capture Molecular Target will be attached to the substrate. The coating material typically binds to a substrate such as glass via electrostatic binding between negative silyl-OH groups on the surface and charged amine groups in the polymers. Poly-1-lysine coated glass slides can also be obtained commercially (e.g., from Sigma Chemical Co., St. Louis, Mo.). Capture Molecular Targets, in defined volumes, are deposited on the coated substrate. The Capture Molecular Targets remain bound to the coated substrate surface non-covalently when an aqueous solution containing DARTs is applied to the array under conditions that allow hybridization or binding of the DARTs to the cognate Capture Molecular Target.

The Capture Molecular Targets attached or immobilized on the substrate can also be non-nucleic acid molecules, such as peptides, proteins or other molecules. Such Capture Molecular Targets can be applied to the substrate by methods known in the art, such as, for example, synthesis on solid phase supports. For example, a substrate can be prepared by derivatizing a starting compound onto a solid-phase support.

Blocking of the Capture Molecular Target and Affinity Target can reduce non-specific interactions between those molecules and between the Capture Molecular Targets, Affinity Targets, and other molecules. For example, the Affinity Substrate can be blocked to reduce non-specific interactions that can occur between the Affinity Targets (e.g., DARTs) and the molecules present on the substrate. Similarly, the Capture Molecular Targets and Affinity Targets can be blocked to prevent non-specific interactions between these molecules.

Agents useful for blocking are typically those that do not inhibit the specific binding interaction between a Capture Molecular Target and an Affinity Target, but that reduce non-specific binding between the Affinity Target and the Capture Molecular Target, and or between other (i.e., non-DART molecules) and the Affinity Target or Capture Molecular Target. Examples of useful blocking agents include, but are not limited to, solutions containing low concentrations of salmon sperm DNA (e.g., 10-500 μM), bovine serum albumin (e.g., 0. 1-5%) and/or fat free milk.

Following blocking, the Affinity Targets are typically contacted with the Capture Molecular Targets. The Capture Molecular Targets can be in solution or on a substrate (i.e., an Affinity Substrate). When the Affinity Targets are contacted with the Capture Molecular Targets, specific binding reactions can occur. Such binding reactions include, for example, CMT:AT, or CMT-AT, binding events. When the Affinity Targets contact with the Capture Molecular Targets, the Affinity Targets can bind covalently or non-covalently to Capture Molecular Targets. Such a CMT:AT interaction can also occur in vivo (e.g., in cells in which Capture Molecular Targets and Affinity Targets are either co-expressed and/or co-delivered).

Another possible binding reaction is a CMT:PS (or CMT-PS) binding event. In such binding reactions, the Capture Molecular Targets are attached or immobilized (either covalently or non-covalently) on a substrate (a PS) to form an Affinity Substrate. Such CMT:PS (or CMT-PS) interactions typically occur in vitro.

A CMT:AT complex can also bind to a Purification Substrate, either covalently or non-covalently. For example, a CMT:AT complex (pre-formed either in vitro or in vivo) can be incubated in the presence of the Purification Substrate. After this incubation, the Capture Molecular Target of the CMT:AT complexes can be covalently coupled to the Purification Substrate.

The Affinity Target can also bind non-covalently to Capture Molecular Targets present in a pre-formed CMT:PS substrate (or CMT-PS substrate). The Affinity Substrate (i.e., CMTs immobilized on a Purification Substrate) can be contacted with the Affinity Targets under various conditions in the presence of a complex mixture harboring, in part, the Affinity Targets. The Affinity Targets non-covalently bind to the Capture Molecular Targets on the Affinity Substrate.

As will be appreciated by the skilled artisan, DARTs, DART components and DART associated molecules can be contacted in a variety of steps to facilitate DART purification. These include, for example, the following: (1) Complex solutions harboring DARTs can be contacted with one or more Capture Molecular Targets (e.g., in the presence of blocking solution) under conditions suitable for forming of a DART:CMT complex. During this incubation DARTs harboring the appropriate Affinity Targets can bind to the Capture Molecular Targets. Alternatively, DART:CMT binding events can occur in vivo in cells in which the Capture Molecular Targets and DARTs are co-expressed and/or co-delivered. (2) Complex solutions harboring CMT:DART complexes can be covalently or non-covalently bound to a Purification Substrate. The CMT:DART complexes (formed either in vitro or in vivo) are contacted in the presence of the Purification Substrate. After contacting, the Capture Molecular Targets of CMT:DART complexes immobilized on the Purification Substrate can, if desired, be covalently coupled to the Purification Substrate. (3) DARTs can bind non-covalently to Capture Molecular Targets present on an Affinity Substrate. The Affinity Substrate can typically be contacted with the Capture Molecular Target in a complex mixture harboring, in part, the DART Affinity Targets. During this contacting, the DARTs can bind non-covalently bind to the Affinity Substrate.

In some embodiments, following the binding reaction, the Capture Molecular Target:Affinity Target complexes can be washed. For example, CMT:AT complexes immobilized on Purification Substrate can be washed to remove molecules that do not specifically bind to the Affinity Substrate and/or Affinity Target. Immobilized DART:CMT complexes can be washed in a solution that does not stimulate a significant amount of dissociation of CMT:DART complexes and/or DART:CMT:PS complexes, but disrupts non-specific interactions between other molecules in the mixture and the complexes. During this wash step, DARTs and/or other molecules that are not specifically bound to the Affinity Substrate are removed, leaving the Affinity Substrate enriched for the relevant Affinity Targets (e.g., DART or Affinity Targets on Darts).

The bound Affinity Targets, or molecules containing the Affinity Targets (e.g., DARTs) can optionally be eluted, collected and/or analyzed. Generally, fractions enriched in ATs can be obtained by specifically eluting Affinity Targets from the Capture Molecular Targets. This can be achieved, for example, by incubating them under conditions that disrupt the CMT:AT or AS:AT interactions, and/or eluting AT:CMT complexes from the Purification Substrate.

DARTs non-covalently bound to a Capture Molecular Target can also be eluted from substrates. This elution can be employed to obtain fractions enriched in DARTs (and any other molecules that are bound to or complexed with the DARTs). DART elution from an Affinity Substrate can be achieved, for example, by dissociating the DART from its corresponding Capture Molecular Target and/or by dissociating a Capture Molecular Target from a Purification Substrate. Once this interaction is disrupted, the DART can be removed from the substrate (and, if desired collected) by washing the substrate in a small volume of the appropriate biological buffer or other solution. Similar methods can employed to selectively elute DART:CMT complexes from Purification Substrates. As will be appreciated by the skilled artisan, the specific elution conditions for disrupting DART:CMT and/or the DART:CMT:PS interactions can depend on the molecular identities of the Affinity Substrate and the Affinity Target mediating the interaction.

The following examples illustrate possible interactions, and general ways in which such interactions can be disrupted during elution, although the present invention is not intended to be limited by or to these examples.

In one example, the interaction is a Molecular Shaft: Capture Molecular Target (MS:CMT) interaction, where the Capture Molecular Target is a nucleic acid including sequences complementary to those present in the Molecular Shaft. The immobilized DART (and corresponding Molecular Shaft) can be eluted from the Affinity Substrate by, for example, raising the temperature above the Tm for the MS:CMT interaction, by adding a site-specific restriction endonuclease that can selectively cut the heteroduplexed nucleic acids mediating the interaction (i.e., the MS-CMT heteroduplex), such that the DART molecule is released from the substrate; and the like.

Another example is a Molecular Point: Capture Molecular Target (MP:CMT) interaction, where the Capture Molecular Target is a protein or other molecule that specifically binds to the Molecular Point of the DART. The immobilized DART (and corresponding Molecular Point) can be eluted from the Affinity Substrate by, for example, adding a large excess of the soluble Capture Molecular Target; by heating and denaturing the interacting molecules; by adding high concentrations of salt and/or detergents to disrupt the interaction; by raising or lowering the pH to disrupt the MP:CMT interaction; and the like.

As will be appreciated by the skilled artisan, similar considerations can allow the elution of non-covalent DART:CMT complexes from a Purification Substrate when CMT:PS binding events are mediated by pairwise interactions between polypeptides, nucleic acids, and/or small molecules.

When extracting DARTs from cell or tissue extracts and/or processing them through affinity purification steps, it is typically preferable to exercise care to preserve the integrity of the DARTs (e.g., the integrity of the nucleic acid and non-nucleic acid components of DART molecules) and to limit DART shuffling.

DART integrity can be preserved, for example, by processing DARTs under conditions in which nuclease and/or protease activities are inhibited and/or reduced. For example, host cells producing DARTS can be nuclease and/or protease-deficient (e.g., by harboring mutations in genes coding for the nucleases and/or proteases). Extracts derived from such host cells can possess reduced amounts of proteolytic and/or nucleolytic enzymes and therefore be less likely to compromise DART integrity. Inhibitors of nuclease and/or protease activities can also be employed. Such inhibitors include, for example, low or high temperatures; nuclease and/or protease inhibitors (e.g., aprotinin, leupeptin, and the like); molecules known to chelate divalent cations required for proteolytic or nucleolytic activity (e.g., EDTA, EGTA, and the like); and/or organic solvents (e.g., ethanol, methanol, acetonitrile, and the like). Such inhibitors typically do not denature or otherwise render inactive the DART components (e.g., Molecular Point, Linkage Polypeptide and/or Molecular Shaft).

To preserve the informational integrity of DART molecules, DART shuffling is typically reduced by purification/enrichment strategies that are carried out under conditions in which DART shuffling is reduced or minimized. Conditions that inhibit DART shuffling include, but are not limited to, those that inhibit the activity of DART Linkage Polypeptide, transcatalytic or transcomplementing polypeptides, and/or inhibit the availability of preMS molecules, postMS molecules, or other nucleic acid substrates. Inhibitors of DART shuffling can include, but are not limited to: (1) high and low temperatures; (2) chelators (e.g., EDTA, EGTA and/or other molecules known to chelate the divalent cations upon which LP activities are dependent); and/or (3) peptide, protein, and/or small organic molecules that interact with Linkage Polypeptides and reduce or inhibit their activity.

DART Affinity Purification

DARTs can be affinity purified to remove molecules that may stimulate DART shuffling. DART affinity purification can entail, for example, separating DARTs from free Linkage Polypeptides, free preMS molecules, and/or free postMS molecules. Non-limiting examples of such affinity purification procedures are provided below.

Separating Free LPS From DARTs

Free Linkage Polypeptides can be separated from DARTs by contacting a solution containing the Linkage Polypeptides and DARTs with an Affinity Substrate that has a large molar excess of nucleic acids (Molecular Targets) that are complementary to DART Molecular Shafts, but that lack the Recognition Sequence Motif for the Linkage Polypeptide. The DARTs, but not free LP molecules, bind to the Affinity Substrate (via MS-MT interactions). The free LP molecules can then be washed away from the Affinity Substrate. The DARTs can optionally be eluted from the Affinity Substrate. Alternatively, if the DARTs and free Linkage Polypeptide differ significantly in size, they can be separated by, for example, dialysis, liquid gel filtration and/or HPLC sizing chromatography.

Separating Free PreMS and/or PostMS Molecules From DARTs

Free preMS molecules can be removed from fractions harboring DART molecules by contacting them with an Affinity Substrate. The Affinity Substrate typically has a large molar excess of an affinity reagent that specifically binds the DART Linkage Polypeptide and/or Molecular Points (e.g., an antibody that binds to the LP or MP), but does not bind with a similar affinity to preMS and/or postMS molecules. Upon contacting the complex mixture with the Affinity Substrate, the DARTs, but not the relevant free nucleic acid molecules, bind the Affinity Substrate (via M?-MT or MP-MT interactions). The free nucleic acid molecules can then be washed away from the Affinity Substrate, and the DARTs are optionally eluted from the substrate. Dialysis, liquid gel filtration chromatography or HPLC sizing chromatography methods can also be used to separate the nucleic acids (preMS and postMS molecules) from the DARTs.

Combinatorial Affinity Purification For DART Enrichment

The methods described hereinabove can be used in a combinatorial protocol fashion to obtain fractions that are enriched in DART molecules, and that lack undesired non-DART molecules. For example, a mixture of DARTs can be contacted with an Affinity Substrate that has Molecular Targets complementary to DART Molecular Shafts. This procedure can remove free Linkage Polypeptides from the mixture.

To separate DARTs from preMS and postMS molecules, a second round of affinity purification, using an Affinity Substrate that binds the DART Linkage Polypeptides and/or Molecular Points can be used. PreMS and postMS molecules which do not bind to the Affinity Substrate, and can be removed by washing. The DARTs are optionally eluted from the Affinity Substrate.

Resolving and Detecting DARTs

In another aspect, methods are provided for detecting DARTs and DART complexes. As discussed herein, DARTs can be resolved and detected using DARTboard technology. DARTs can also be resolved by, for example, traditional gel or capillary gel electrophoresis, reverse or fluid phase chromatography (e.g., anion exchange, cation exchange, hydroxyapatite chromatography, and the like), liquid chromatography or high performance liquid chromatography (HPLC). Following resolution, DARTS can be transferred to a substrate (e.g., nitrocellulose or PVDF membranes) for subsequent detection.

DARTs can be detected by methods known to those of skill in the art, including but are not limited to, antibody-mediated detection, radiometric detection, labeling of DARTs and analytical chemical detection. Antibody-mediated detection can be used to detect DARTs. For example, the Molecular Points and/or affinity tags on DARTs (e.g., 6×HIS, FLAG, MYC and the like) can be detected using antibodies against the Molecular Points or the affinity tags. Antibody binding to DARTs can be detected, for example, by incorporating a label on the antibody, such as, for example, a luminescent or fluorescent molecule, a radioactive label, an enzyme, biotinyl groups, and the like.

DARTs can be radiolabeled for radiometric detection. For example, the DART Molecular Point, Molecular Shaft and/or Linkage Polypeptide, or combinations of these can be radiolabeled. The presence of a DART can then be detected using standard radiometric detection methods.

DARTs can also be labeled by incorporating a label on or into the DART. Such labels can include luminescent or fluorescent molecules, enzymes, biotinyl groups, and the like. In some embodiments, DARTS can be differentially labeled (e.g., using different fluorescent labels) so that the DARTs can be distinguished. Although confocal detection systems are typically used for data collection, according to one embodiment a high resolution CCD camera system is utilized for data collection.

DARTs can also be detected by hybridizing a Probe Molecular Target, such as a labeled nucleic acid to a DART Molecular Shaft. DARTs can also be detected by analytical chemical approaches, such as, for example, HPLC and/or mass spectrometry. For overview of detection by mass spectrometry, see Carr and Annan, “Overview of Peptide and Protein Analysis by Mass Spectrometry,” in Current Protocols in Molecular Biology, Ausubel et al., eds., John Wiley & Sons, Inc., 1997, 10.21.

DARTboards

The development and analysis of new drugs depends on the discovery of compounds that bind specifically to biologically important molecules. However, drug candidate discovery can require screening very large numbers of compounds (e.g., 1000, 10,000, 10,000,000, or much more). Therefore, tools for screening compounds efficiently are of particular importance to the drug discovery and development process. Of particular importance are tools that allow for the capture of relevant intermolecular interactions in cells before subsequent resolution and detection in vitro. DARTboard protein arrays can be used in a wide range of commercial applications, including the analysis of protein-protein binding events, protein-drug binding events, detection of protein modifications, and other uses.

One method for screening a large number of compounds in vitro is to fix possible targets of these compounds onto a supported substrate. Such targets can include proteins that may or may not have been exposed to protein or non-protein pharmaceutics in vitro or in vivo. One aspect of DART technology provides for DARTboards, an array tool which, in certain embodiments, can provide an ordered and indexed array of DARTs on a substrate in which each DART species occupies a discrete locus on the supported substrate (e.g., 96-well microtiter plate).

An “array” includes, but is not limited to, a fixed pattern of immobilized objects on a solid surface or membrane. DARTboards provide methods for generating protein arrays, in which the proteins are not necessarily covalently bound to the array. The specificity of non-covalent binding interactions between DARTs and immobilized Capture Molecular Targets can be used to fabricate DARTboards.

Typically, a DARTboard comprises at least two DARTs bound, covalently or non-covalently, to an Affinity Substrate (i.e., a Capture Molecular Target attached or immobilized on a Purification Substrate). Any component of the DARTs can bind the Affinity Substrate, such as, for example, a Molecular Point, a Molecular Shaft, a Linkage Polypeptide or another molecule covalently or non-covalently attached to any of these. The DART is typically bound or hybridized to the substrate by the Molecular Shaft.

By selection of the Affinity Substrate and/or the DART, DARTs can be specifically targeted to a defined context on a substrate. The following examples describe possible interactions between DARTs and Affinity Targets.

In one embodiment, the Molecular Shaft of a DART, or DARTs, can be used to target the DARTs to specific locations on Affinity Substrates (also referred to as MS-mediated DART targeting). The Affinity Substrate has Capture Molecular Targets bound to the substrate. The Capture Molecular Target can be, for example, a nucleic acid with sufficient complementary to the Molecular Shaft of the DART that a specific MS:CMT duplex can form under suitable hybridization conditions. If the substrate has a plurality of different Capture Molecular Targets, then the DARTs (e.g., in a complex mixture of DARTs) will typically bind to different, complementary nucleic acids on the substrate. Further, because the Molecular Point and Linkage Polypeptide are covalently linked to the Molecular Shaft, they are also targeted to the same location on the substrate. Such targeting can be used to identify specific DARTs in a complex mixture of DARTs, and/or to target specific biological and/or chemical (e.g., enzymatic) activities of DARTs to the substrate.

DARTboards can be prepared according to methods known in the art. In a typical embodiment, a DARTboard is prepared by first preparing an Affinity Substrate having at least two different Capture Molecular Targets on the substrate. Nucleic acids can be attached or immobilized on the substrate that may include, for example, glass slides, silicon wafers, the wells of a microtiter plate, nitrocellulose membranes, nylon membranes, PVDF membranes, or any other solid or semi-solid surface on which Capture Molecular Targets or other molecules can be attached or immobilized.

In another embodiment, DARTboards can be prepared by contacting an MP-LP pair with Capture Molecular Targets (CMT) comprising preMS sequences. The CMTs are typically immobilized on solid surfaces such as, for example, glass slides, silicon wafers, the wells of a microtiter plate, agarose beads, nitrocellulose membranes, nylon membranes, PVDF membranes, or any other solid or semi-solid surface. Contacting an MP-LP fusion pair with the Capture Molecular Target on the Affinity Substrate results in linkage of these agents to generate a DART on the surface to which the Capture Molecular Target is attached. In one embodiment, a robot, mechanical, manual, or other deposition device can be used to contact the MP-LP pair with Capture Molecular Targets deposited on a substrate. In another embodiment, MP-LP fusion pairs can be incubated with Capture Molecular Targets deposited on a substrate using protocols known to those skilled in the art. Each MP-LP pair will target and link to Capture Molecular Targets comprising its cognate preMS sequences. One of skill in the art can readily manipulate the conditions to allow the linkage of the MP-LP pair and the preMS. (See-infra for an example of the chemical and physical conditions suitable for linkage of an MP-LP pair in which the MP is VirD2 and the preMS comprises a VirD2 RS.)

Substrates can further include any solid or semi-solid surface including, without limitation, any chip (for example, silica-based, glass, or gold chip), glass slide, membrane, bead, solid particle (for example, agarose, sepharose, or magnetic bead), column (or column material). A variety of materials can be used as the solid support. Examples of such materials include polymers (e.g., plastics), aminated surfaces, gold-coated surfaces, nylon membranes, polyacrylamide pads deposited on solid surfaces, silicon, silicon-glass (e.g., microchips), silicon wafers, and glass (e.g., microscope slides). Microchips, and particularly glass microchips, represent a typical supported substrate surfaces.

The substrates can be fabricated from any of a variety of materials. In certain embodiments, the materials from which the substrate may be fabricated can exhibit a low level of non-specific binding during hybridization events. In many situations, a material that is transparent to visible and/or UV light can be employed. For flexible substrates, suitable materials include: nylon, both modified and unmodified, nitrocellulose, polypropylene, and the like, where a nylon membrane, as well as derivatives thereof, may be particularly useful in this embodiment. For rigid substrates, suitable materials include: glass; fused silica, silicon, plastics (for example, polytetrafluoroethylene, polypropylene, polystyrene, polycarbonate, and blends thereof, and the like); metals (for example, gold, platinum, and the like). The substrate surface onto which DARTs are delivered or deposited can be smooth or substantially planar, or have irregularities, such as depressions or elevations.

For DARTboard fabrication, Capture Molecular Targets are typically arranged in an ordered and indexed array on the substrate, such that different nucleic acids are present at different loci on the substrate. In a typical embodiment, a DARTboard can be formed in which the DARTs are self-referential (i.e., the Molecular Shafts of the DARTs encode the corresponding covalently coupled Molecular Points). The Capture Molecular Targets on the DARTboard are nucleic acids complementary to the DART Molecular Shaft sequences. Such a DARTboard can beneficially be formed by self-assembly of DARTs on a DNA microarray. The fully formed DARTboard can be an ordered array of Molecular Points, each localized to a distinct locus on the DARTboard, and therefore the identity of each Molecular Point (and DART) can be rapidly and unambiguously identified by reference to its covalently attached Molecular Shaft.

As will be apparent to one skilled in the arts, the Capture Molecular Targets present on the Affinity Substrate can be modified before they are attached to the surface. For example, one or more non-nucleosidic spacers, such as polyethylene oxide, can be added to the terminus of the nucleic acid. The nucleic acid spacers provide physical separation between the nucleic acid and the solid surface and prevent interaction of the proteins with the support surface. In a further aspect, the nucleic acid Capture Molecular Targets immobilized on the surface can include a modified base, such as 5-propyne pyrimidine. It can also include an internucleotide analog (such as 3′-phosphoramidate) or a carbohydrate modification (such as a 2′-O-methyl group). In a typical embodiment, the nucleic acid sequences immobilized on the surface can include a reactive moiety for covalently linking the DART to the nucleic acid immobilized on the surface (for example, by photo-crosslinking).

In one example, a DARTboard can be formed on a substrate on which two, three, four, or more Capture Molecular Targets are immobilized at distinct loci on the substrate to form an Affinity Substrate. If the Capture Molecular Targets are different, then DARTs can be applied to the substrate in a mixture. For example, if DART 1 (D1) binds to Capture Molecular Target 1 (CMT1), and DART 2 (D2) binds to Capture Molecular Target 2 (MT2), then if CMT1 and CMT2 are attached or immobilized at distinct loci on the array, the DARTs will bind at distinct positions on the array. D1 will bind to CMT1, and D2 will bind to CMT2.

Molecular Shaft binding to Capture Molecular Targets can occur through base pairing (for example, through Watson-Crick base pairing, pseudo Watson-Crick base pairing involving modified bases, or Hoogsteen base pairing) between the Molecular Shaft of a DART and a complementary immobilized nucleic acids present on the support substrate, or can occur through any other type of sequence-dependent recognition and binding of the immobilized nucleic acid (including, without limitation, polyamide-mediated nucleic acid groove binding or specific binding by nucleic acid-binding proteins such as transcription factors). The result of the binding interactions between the DARTs and the immobilized Capture Molecular Targets can be a defined, an ordered and indexed array of proteins attached to a solid support.

In one embodiment, contacting of the DARTs to the array can be performed by manual spotting, robotic spotting, and the like. For example, D1 can be bound to CMT1 by a separately contacting D1 with CMT 1. Such separate contacting can be performed using a substrate that has individual cells, wells, and the like (e.g., a 96 well microtiter plate). Similarly, D2 can be separately contacted with CMT2. Typically, only minor amounts of D1 are bound to CMT2, and vice versa. The net result is that D1 and D2 co-localize with CMT1 and CMT2, respectively, and therefore upon depositing D1:CMT1 (or D1-D2) and D2:CMT2 (or D2-CMT2) complexes onto the supported substrate, an ordered and indexed array of DARTs (D1 and D2) is formed (i.e., a DARTboard). In another embodiment, D1 can be contacted with the Capture Molecular Target already present on the supported substrate by a separately contacting D1 with the Capture Molecular Target at one locus. Similarly, D2 can be separately contacted with the Capture Molecular Target at a different locus. The net result is that D1 and D2 form an ordered and indexed array of DARTs.

In another embodiment, the Capture Molecular Target can be a complementary to the Molecular Shaft of many DARTs (e.g., the Capture Molecular Target is complementary to a portion of the Molecular Shafts of the DARTs). As will be apparent to one skilled in the arts, these nucleic acid Capture Molecular Targets typically have at least 5 to 30 nucleotide units, and can have more than 20 nucleotide units. Considerations for the selection of the exact sequence for a particular immobilized nucleic acid include, for example, melting temperature (Tm), interference from competing target sequences, and potential secondary structure in the target sequence. In some embodiments, each unique nucleic acid Capture Molecular Target has about the same Tm, so a single hybridization and washing temperature can be used successfully for all Molecular Shaft-Capture Molecular Target pairs. Commercially available computer programs can be used to help identify sets of capture probes with similar thermodynamic properties based on, for example, nearest neighbor treatments.

In some embodiments, the array can be pre-treated with a blocking reagent to prevent or reduce non-specific binding of DARTs to the array. The blocking reagent can also reduce or prevent internal interference of some DART components with others DART components. Examples of appropriate blocking reagents include, for example, neutral buffers containing lowfat milk, bovine serum albumin (e.g., a 3-5% solution), salmon sperm DNA (e.g., 10-100 μg/ml), and the like. One or more DARTs are then contacted with the array. The DARTs can be contacted in solution that includes the blocking reagent to create a “blocked DART mixture.” The blocked DART mixture can optionally include inhibitory concentrations of a reagent known to prevent the covalent linkage reaction of the DART Linkage Polypeptides. Such reagents can include, for example, those that chelate divalent cations (e.g., EDTA or EGTA), nucleic acids possessing specific sequences that have the capacity to inhibit the covalent linkage reaction, exonucleases, and the like.

The blocked DART mixture is contacted with the array for a sufficient period to allow individual DARTs to bind specifically to the complementary Capture Molecular Targets on the array. During contacting, the DARTs that bind to the array become localized to specific loci on the array. Once contacting is complete, the array is optionally washed to remove excess blocked DART mixture, non-specifically bound DARTs, and the like. Following washing, the DART(s) bound to the array can optionally be cross-linked to the corresponding Capture Molecular Target using a variety of common cross-linking procedures, including UV treatment or commercially available cross-linking reagents. Such covalently linked DARTs provide particularly robust and versatile protein arrays for the screening methods described herein. Covalently linked DART arrays may be generated by any standard approach known to those skilled in the arts.

There are a wide variety of means for detecting DARTs on a DARTboard. The detection means can vary, depending upon the specific molecular composition of the immobilized DARTs. Examples of detection methods include, for example, specific Probe Molecular Target contacting the DARTs in the DARTboard. For example, antibody-mediated detection (e.g., using antibody against a Molecular Point or Linkage Polypeptide), antibody against an affinity tag (e.g., the 6×HIS or FLAG epitopes), and the like; direct detection of bound DARTs using analytical chemical methods (e.g., mass spectrometry); radiometric detection of radiolabeled DARTs; detection of labeled DARTs (e.g., fluorescent labels, enzymatic labels, chemiluminescent labels, and the like).

DARTs can also be eluted and analyzed from a DARTboard by methods known to the skilled artisan, including those described herein. Elution can be used to purify DARTs (and any other molecules that the DARTs may, in turn, be complexed with), or to remove DARTs from a substrate so that the substrate (and its immobilized Capture Molecular Targets) can be re-used.

DART elution from a supported substrate can be performed by disrupting the interaction between the DART and its corresponding Capture Molecular Target. Once this interaction is disrupted, the uncoupled DART can be removed from the substrate (and, if desired collected) by, for example, washing the substrate in an appropriate volume of excess buffer. The specific means employed to disrupt the DART/Capture Molecular Target interaction can depend on the molecular identities of the components mediating the interaction. For example, a Molecular Shaft: nucleic acid interaction can be disrupted by raising the temperature (e.g., boiling) above the Tₘ for the MS:CMT interaction or by adding a nuclease (e.g., a restriction endonuclease) that possesses the capacity to selectively cut the duplexed nucleic acids mediating the interaction (that is, the MS:CMT heteroduplex) such that the DART is released from the substrate. A Molecular Point:protein Capture Molecular Target interaction can be disrupted by, for example, adding a large excess of the soluble Capture Molecular Target; changing the temperature (e.g., heating or boiling) to denature the interacting protein; adding high concentrations of salt or detergents or other chemicals to disrupt the intermolecular interactions; raising or lowering the pH; and/or any combination of the above.

DARTs can also be eluted from the supported substrate by employing a light-sensitive linker. This linker can be an integral part of the immobilized Capture Molecular Target affixed to the supported substrate, or a component of DART Molecular Shaft components. DART molecules possessing, or contacting molecules possessing, this cleavable linker can be released upon linker cleavage. A beam of the appropriate wavelength can be employed to cleave the linker, thus releasing the desired DART. Following release from the DARTboard surface by this or any of the above methods, the DART can be specifically recovered and manipulated, for example, using PCR, and further characterized.

DARTboards can be used in a wide variety of different ways. For example, DARTboards can be screened for binding of a variety of Probe Molecular Targets (e.g., small molecules, proteins, nucleic acids, antibodies, and the like) to the DARTs. The Probe Molecular Targets can be labeled or unlabeled. In another embodiment, the Molecular Points of the DARTs present in a DARTboard can be screened for biochemical activity. Suitable biochemical activity assays include those that generate a chromogenic, luminescent or other detectable product. Such assays are typically performed using micro-reactions performed in droplets spotted at a locus on the array, in a multi-well format, and the like. The location of product on the DARTboard can be detected with, for example, photon detection or auto-radiographic techniques. Through knowledge of the sequence of the material at the location where the relevant activity is detected, it is possible to quickly determine which DARTs possess the desired activity. This technique can be used to screen large numbers molecules quickly and economically.

In another embodiment, DARTs present on the DARTboard can be exposed to conditions that may modify the chemical composition, or alter the physicochemical integrity of some or all of their members. Conditions that may lead to such modifications include, but are not limited to, treatments with specific proteases, kinases, phosphatases, and the like. Specific DART targets for these modifications may be detected using a variety of methods described herein or apparent to one skilled in the arts.

In another embodiment, DARTboards can be used for recovering and analyzing the molecular composition of DARTs that have been exposed to different in vitro or in vivo (e.g., cellular) conditions. For example, DARTs synthesized in, or delivered to, target cells can capture protein modifications (e.g., proteolysis, phosphorylation, and the like), protein-compound, or protein-drug interactions in these cells under particular circumstances. These DARTs can then be rapidly analyzed by creating DARTboards. Similarly, DARTboards can be used to monitor the expression of genes, nucleic acids or proteins in a cell. DARTboards can also be used for large-scale, high thru-put screening assays to identify drugs altering cellular protein modification or degradation patterns.

In another embodiment, DARTboards can be employed to identify previously unknown protein-protein interactions, or to verify or explore known or hypothesized protein-protein interactions. A protein Probe Molecular Target can be detectably labeled; for example, with a radioisotope, chromophore, fluorophore, or chemiluminescent species, then incubated with the DARTboard. After any excess Probe Molecular Target is washed away, the DARTboard can be analyzed for signal from the label. Detection of a signal can indicate interaction of the labeled Probe Molecular Target with the relevant DART present on the DARTboard. As will be apparent to those skilled in the arts, protein Probe Molecular Targets interacting with the DARTboard may also be detected using surface plasmon resonance, mass spectrometry, or other methods.

In another embodiment, DARTboards can be employed for detecting an interaction between a protein and a drug or other compound. This result may be achieved, for example, by first subjecting a pre-fabricated DARTboard to conditions that allow molecular interactions between DART components and the drug or compound, and then, analyzing these DARTboard components for the presence of the drug or compound. The same approach can also be used to screen DARTboards for DART interactions with Probe Molecular Targets, such as agonists and antagonists for intact of portions of cell membrane receptors, toxins and venoms, viral epitopes, hormones, hormone receptors, peptides, enzymes, enzyme substrates, cofactors, drugs (e.g., opiates, steroids, etc.), lectins, sugars, oligonucleotides (such as in hybridization studies), nucleic acids, oligosaccharides, proteins, benzodiazapines, prostaglandins, beta-turn mimetics, monoclonal antibodies, and the like.

In another embodiment, a DART or populations of DARTs may contact the compound or drug before these DARTs are employed to fabricate a DARTboard. This DARTboard can then be analyzed for the presence of the compound or drug. The analysis can provide information about interactions between the relevant DART components and the compound. Because for each of the above applications the compound can be labeled, a rapid assessment about the compound's DART binding partners can be made. Compounds that can be screened using these methods include, without limitation, proteins, small molecule drugs, other small molecules, carbohydrates, lipids, nucleic acids, and the like.

In another embodiment, DARTboards can be used for molecular diagnostic applications, including the analysis of serum and other samples, for the presence of viruses, bacteria, chemicals, or disease-associated molecules, as more fully described below.

In another embodiment, DARTboards can be employed for the immobilization of cells displaying cell surface molecular determinants that allow them to be ‘captured’ by DART components present on the DARTboard. These molecular determinants may include antigens recognized by antibody epitopes present in Molecular Shaft components of DARTS present on the DARTboards.

Modulation of LP Linking Activity

LPs can comprise attractive pharmaceutical targets (e.g. viral replication proteins such as Polio virus VpG, conjugation enzymes such as TraI, Agrobacterium virulence proteins such as VirD2, rolling circle replication enzymes such as RepC and the like). For this reason, assays for LP activity can be useful for identifying drugs that stimulate or antagonize the activity of the LP itself. For example, in specific embodiments, compounds are screened for their ability to prevent DART formation in the presence of components that would form a DART in the absence of the test compound. Thus, the present invention encompasses methods for assaying the linking activity of an LP molecule with a Capture Molecular Target comprising preMS sequences. The linking activity can be assayed under different physical and/or chemical conditions to identify optimal linking conditions. Alternatively, the linking activity of the LP can be assayed in the present of a test compound to determine if the test compound disrupts the LP's linking activity. Contacting the LP with the Capture Molecular Target on the affinity substrate will result in covalent linkage of these elements. In one embodiment, a robotic mechanical, manual, or other deposition method known to the skilled artisan is used to contact the LP molecules with Capture Molecular Targets deposited on a substrate. In another embodiment, LP molecules are incubated with Capture Molecular Targets deposited on a substrate. Chemical and physical conditions for this contacting are typically chosen that permit linkage of the MP-LP pair and the preMS. In this case, each LP molecule can target and link to a Capture Molecular Target comprising its cognate preMS sequences. These methods can provide an assay for LP linking activity under various contacting conditions. In one embodiment, a drug candidate is added to the contacting conditions to assay its ability to prevent or promote LP linking activity. In another embodiment, mutations or other physical or chemical alterations to the LP are assayed for their effect on LP linking activity. In further embodiments, modifications of salt, detergent, and/or buffer concentrations are assayed.

DART Molecular Interaction Assays

In another aspect, methods are provided for detecting the interaction of DARTs with other molecules. Such interactions can include the interaction of DART Molecular Points and/or Molecular Shafts with other molecules (e.g., the Molecular Point or Molecular Shaft of another DART). Such methods can allow the detection of non-covalent interactions between DARTs and between DARTs and other molecules. The resulting non-covalent, multimeric complexes can contain information that is useful for determining the functions of DART components (e.g., protein or nucleic acid) and the biology and/or chemistry of the system in which they function. Identifying the components that form such a multimeric complex can also help reveal the binding partners of DART components. For example, in one aspect, DARTs can be used to analyze the order of interaction of components of a signal transduction pathway or provide structural information about the interaction of subunits of a multimeric complex. The uses of a “Two-hybrid” system (infra) illustrate the uses of DART molecular interactions inventions comprising DARTex and DARTdance.

An approach to identifying polypeptide sequences which bind to a predetermined polypeptide sequence has been to use a so-called “two-hybrid” system, wherein the predetermined polypeptide sequence is present in a fusion protein (see, e.g., Chien et al., 1991, Proc. Natl. Acad. Sci. (USA) 88:9578). This approach identifies protein-protein interactions in vivo through reconstitution of a transcriptional activator (see, e.g., Fields and Song, 1989, Nature 340:245), the yeast Gal4 transcription protein. Typically, the method is based on the properties of the yeast Gal4 protein, which consists of separable domains responsible for DNA-binding and transcriptional activation. Polynucleotides encoding two hybrid proteins, one consisting of the yeast Gal4 DNA-binding domain fused to a polypeptide sequence of a known protein and the other consisting of the Gal4 activation domain fused to a polypeptide sequence of a second protein, are constructed and introduced into a yeast host cell. Intermolecular binding between the two fusion proteins reconstitutes the Gal4 DNA-binding domain with the Gal4 activation domain, which leads to the transcriptional activation of a reporter gene (e.g., lacZ, HIS3) which is operably linked to a Gal4 binding site. Typically, the two-hybrid method is used to identify novel polypeptide sequences which interact with a known protein (Silver and Hunt, 1993, Mol. Biol. Rep. 17:155; Durfee et al., 1993, Genes Devel. 7:555; Yang et al., 1992, Science 257:680; Luban et al., 1993, Cell 73:1067; Hardy et al., 1992, Genes Devel. 6:801; Bartel et al., 1993, Biotechniques 14:920; and Vojtek et al., 1993, Cell 74:205). However, variations of the two-hybrid method have been used to identify mutants of a known protein that affect its binding to a second known protein (see, e.g., Li and Fields, 1993, FASEB J 7:957; Lalo et al, 1993, Proc. Natl. Acad. Sci. USA 90:5524; Jackson et al., 1993, Mol. Cell. Biol. 13:2899; and Madura et al., 1993, J. Biol. Chem. 268:12046). Two-hybrid systems have also been used to identify interacting structural domains of two known proteins (see, e.g., Bardwell et al., 1993, Med. Microbiol. 8:1177; Chakraborty et al., 1992, J. Biol. Chem. 267:17498; Staudinger et al., 1993, J. Biol. Chem. 268:4608; and Milne and Weaver, 1993, Genes Devel. 7:1755) or domains responsible for oligomerization of a single protein (see e.g., Iwabuchi et al., 1993, Oncogene 8:1693; Bogerd et al., 1993, J. Virol. 67:5030). Variations of two-hybrid systems have been used to study the in vivo activity of a proteolytic enzyme (see, e.g., Dasmahapatra et al., 1992, Proc. Natl. Acad. Sci. USA 89:4159). Alternatively, an E. coli/BCCP interactive screening system (Germino et al., 1993, Proc. Natl. Acad. Sci. USA 90:933; Guarente, 1993, Proc. Natl. Acad. Sci. USA 90:1639) can be used to identify interacting protein sequences (i.e., protein sequences which heterodimerize or form higher order heteromultimers). Sequences selected by a two-hybrid system can be pooled and shuffled and introduced into a two-hybrid system for one or more subsequent rounds of screening to identify polypeptide sequences which bind to the hybrid containing the predetermined binding sequence. The sequences thus identified can be compared to identify consensus sequence(s) and consensus sequence kernals.

As can be appreciated from the disclosure herein, the present DART molecular interactions inventions have a wide variety of applications. Accordingly, the preceding references are offered by way of illustration, not by way of limitation.

Dartex

In one aspect, DARTex can be used to analyze molecular interactions involving DARTs. DARTex provides a means for the analysis of DART interactions by the transfer of all or part of a Molecular Shaft from one DART to another DART. This exchange reaction can be mediated by the Linkage Polypeptide of one DART interacting with a Recognition Sequence Motif on the Molecular Shaft of a second DART. DARTex can occur, for example, when the Molecular Points of two DARTs (e.g., Molecular Point 1 (MP1) and Molecular Point 2 (MP2)) bind to one another. DARTex therefore provides a means of detecting or recording the interaction of the Molecular Points by the transfer of Molecular Shaft (i.e., nucleic acid sequences) from one DART to another. The DARTex strand exchange assay can be used to detect DART interactions in vivo or in vitro.

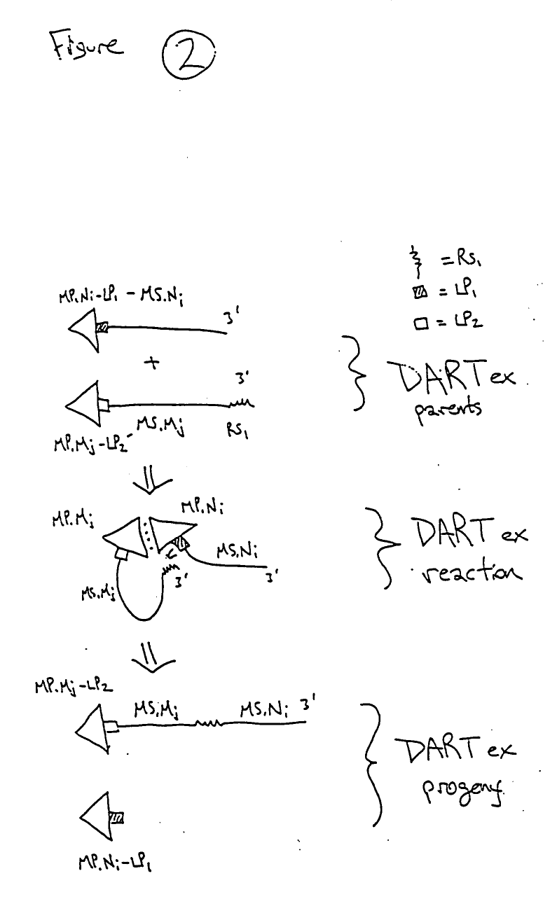

In DARTex, the two DARTs, designated DARTex “parents” (e.g., DART.N and DART.M) (See, e.g., FIG. 2), interact resulting in transfer of one of the Molecular Shaft of one DART to another DART. The resulting molecules, designated DARTex progeny, are derived from DART.N and DART.M DARTex parents. One of the DARTex progeny contains nucleic acid derived from the Molecular Shafts of both parents. In one aspect, the other progeny does not contain any parental nucleic acid.

For example, two sets of non-identical DART species, DART.M and DART.N species can be used. DART.M includes MP.M-LP2-MS.M-RS1. MP.M is the Molecular Point, and LP2 is the Linkage Polypeptide, and MS.M is the Molecular Shaft of DART.M. The MS.M Molecular Shaft typically will comprise sequences that encode MP.M. RS1 is a Recognition Sequence Motif recognized by LP1 of DART.N.

DART.N includes MP.N-LP1-MS.N. MP.N is the Molecular Point, LP1 is the Linkage Polypeptide, and MS.N is the Molecular Shaft of DART.N. The MS.N Molecular Shaft typically will comprise sequences that encode MP.N.

For DARTex applications, MS.N and/or MS.M may comprise DNA, RNA, or an RNA-DNA hybrid molecule. As will be also appreciated, it will often be useful to include primer annealing sites within MS.N and/or MS.M that permit subsequent PCR amplification, sequencing, restriction enzyme digestion, hybridization, cloning, or other manipulation of nucleic acids comprising these sequences, including but not limited to DARTs and DARTex progeny.

To perform DARTex, the Linkage Polypeptide of DART.N, LP1, is selected to recognize RS₁. The second DART, DART.M, usually does not contain a Linkage Polypeptide competent to link itself to RS₁. For example, DART.M can have a different Linkage Polypeptide or a Linkage Polypeptide incapable of performing a DARTex reaction. For example, LP1 can be VirD2 and LP2 can be TraI. In this example, RS₁ is a permutation of the VirD2 modified Recognition Sequence Motif (5′ TATATCCTG 3′ (SEQ ID NO: 31)), and the 3′ guanine of RS, coincides with the 3′ terminus of MS.M.

Referring to FIG. 2, in a DART strand exchange reaction, when Molecular Points MP.M and MP.N bind non-covalently, they bring LP1 of DART.N in close proximity to RS₁ of DART.M. LP1 of DART.N interacts with RS₁ to cause a non-reciprocal exchange of nucleic acids, or ligation, resulting in the formation of two new DARTex progeny molecules. In one of the progeny, MP.M-LP2 is now covalently linked to MS.M and to MS.N in the following configuration: MP.M-LP2-MS.M-RS₁-MS.N. The second progeny molecule MP.N-LP1, is typically not covalently linked to either MS.M or MS.N. As will be appreciated by the skilled artisan, the abundance of the MP.M-LP2-MS.M-RS₁-MS.N progeny molecules will be a function of the frequency of DART.N encounters with DART.M, the affinity of the parent DARTs for one another, the efficiency of the LP1-catalyzed ligation reaction, and the like.

DARTex can be used to identify a variety of interactions between DARTs. For example, DARTex can be used to determined whether the Molecular Point of DART.N interacts with (e.g., binds) the Molecular Point of DART.M. In such an assay, two species of DARTex progeny are typically produced: MP.M-LP2-MS.M-RS₁-MS.N and MP.N-LP, if the parental DARTs interact and undergo the DARTex reaction.

DARTex can also be used to identify the binding partners of the Molecular Point of DART.N in a library of DART.Mᵢ species. Such an assay can be used to identify one or more members of the library, DART.Mᵢ molecules, that bind to or otherwise interact with DART.N. Possible DARTex progeny typically include, for example, MP.Mᵢ-LP2-MS.Mᵢ-RS1-MS.N and MP.N-LP.

In another example, a library of different DART.Ni molecules are incubated with a second library of DART.Mj molecules. As will be appreciated, a variety of progeny DARTs can be generated, depending on the interactions between the DARTs of each library. Generally, the progeny can include MP.Mⱼ-LP2-MS.Mⱼ-RS₁-MS.Nᵢ and MP.Nᵢ-LP. The different MP.Mⱼ-LP2-MS.Mⱼ-RS₁-MS.Nᵢ progeny identify the MP interactions. For example, if MP.N₃-LP₁-MS.N₃ interacts with MP.M₅-LP2-MS.M₅-RS₁, the resulting DARTex progeny will include MP.N₅-LP2-MS.M₅-RS₁-MS.N₃. If MP.N₈-LP₁-MS.N₈ interacts with MP.M₁₂-LP-MS.M₁₂-RS₁, the resulting progeny will include MP.M₁₂-LP-MS.M₁₂-RS₁-MS.N₈. The identities of the two MP domains that interact are revealed in the sequences of the MS.M-RS₁-MS.N fusion products of the DARTex reaction.

In other embodiments, DARTex can be performed to detect the interaction of DART.N species with a Molecular Shaft of DART.M species. Alternatively, DARTex can be performed to detect the interaction between a molecule bound to DART.N species or DART.M species, and with which the other DART species can interact.

A DARTex reaction is typically performed by contacting at least a first DART.Nj species and at least a second DART.Mi species under conditions where the Molecular Points can interact and where LP1 is not capable of performing a covalent linkage reaction (e.g. in the presence of EDTA). The DARTex reaction can be performed in vitro or in vivo. Following contacting and any desired wash steps, the DARTex reaction is typically triggered by promoting the linkage activity of LP1. This is typically achieved by addition of an inducing agent, such as a divalent cation (e.g., magnesium) or by the removal of an inhibitory agent (e.g. EDTA). Generally, the DARTex reaction produces MP.Mᵢ-LP2-MS.Mᵢ-RS₁-MS.Nⱼ and MP.Nⱼ-LP progeny. The DARTex reaction can be stopped by conditions (e.g., EDTA or increased temperature) that inhibit LP1 activity.

As will be appreciated, DARTex can be employed to monitor the interactions between any components of DART.M and any component of DART.N. Furthermore, one or more DART or non-DART molecules may be involved and/or required for the DART.M:DART.N binding event and may therefore serve a bridging function between DART.M and DART.N. Consequently, DARTex may be employed to monitor the interactions of DARTs with DARTs, DARTs with non-DARTs, and non-DARTs with non-DARTs.

Typically, DART shuffling is prevented to reduce undesirable reaction byproducts. Such byproducts can arise when the DARTex progeny MP.Nⱼ₁-LP1 performs a covalent linkage reaction with MP.Mᵢ-LP2-MS.Mᵢ-RS₁-MS.Nⱼ₂ to generate MP.Nⱼ₁-LP1-MS.Nⱼ₂, a DART progeny molecule in which the linked MP and MS components no longer retain a useful informational relationship. This shuffled DART might further interact with another molecule via MP.Nⱼ₁ to initiate a second cleavage reaction and further generate a new set of undesired DARTex products. Thus, at the completion of a DARTex reaction it is preferable that the DART progeny remain unreacted (i.e., they are only the progeny of the DART parents, not progeny of progeny) and do not undergo DART shuffling.

Methods for preventing DART shuffling during DARTex reactions include, but are not limited to using a RS1 motif that will act as a Recognition Sequence Motif for LP1 to form the MP.Mⱼ-LP2-MS.Mⱼ-RS1 -MS.Nᵢ product, but will not permit the cleavage of this reaction product by LP1 in other DARTs, using agents (e.g., magnesium and EDTA) and/or reaction conditions (e.g., temperature) that regulate LP1 activity to ensure that only one round of DARTex reactions occurs, limiting the concentration of or diluting the DARTex parents so that only a single round of DARTex reactions is likely to occur during the reaction time, and the like (see supra).

Once the DARTex reaction is completed, the progeny can be resolved and/or detected by any of the methods described herein. For example, the identity and conformation of the progeny can be determined by PCR, nucleic acid hybridization, and the like. If DARTex is performed in vivo, it can also be useful to at least partially purify the DART progeny from other non-DART molecules using the methods described herein.

As will be appreciated, higher order DARTex reactions involving two DARTs and one or more DARTs and/or non-DART molecules can also be performed. Similarly, DARTex reactions can be performed to detect the interaction of a component of one DART species (e.g. MP.N, LP1, and MS.N) with a component of another DART species (e.g. MP.M, LP2, and MS.M.).

In another aspect, the DARTex binding reaction is performed under conditions where one component of the DARTex complex (e.g., DART.N or DART.M or another molecule bound to either DART.N or DART.M) is bound to an affinity substrate. As the skilled artisan will appreciate, the DARTex complex bound to the affinity substrate may be washed to remove unbound molecules (e.g., DNA, RNA, proteins, peptides, small molecule drugs and the like), salts, buffers, detergents, and the like, or to add molecules, buffers, detergents and the like. In a typical embodiment, EDTA is removed and magnesium is added to trigger the LP1-mediated linking reaction.

In another embodiment, DARTex is performed with one DART (e.g. DART.M, comprising MPi-LP1-MSi) and a substrate to which an identity nucleic acid and a Molecular Target are bound in close proximity. The identity nucleic acid typically comprises an Identity sequence and an LP1 recognition sequence that permits LP1 mediated ligation of the identity nucleic acid and MSᵢ. Each different Molecular Target corresponds to a different Identity sequence. The particular Molecular Target can be identified following analysis of DARTex progeny by virtue of the Identity sequence.

The substrate can be designed to allow LP₁ to ligate the identity nucleic acid and MSᵢ when DART.M is bound to the Molecular Target corresponding to the identity nucleic acid. Typically, LP1 cannot ligate MSᵢ to an identity nucleic acid that does not correspond to the Molecular Target to which the DART is bound.

In one embodiment, the different Molecular Targets are bound to different agarose beads comprising an identity nucleic acid and tag combination that is typically unique to that Molecular Target. In one aspect, the Molecular Target comprises a polypeptide. In another embodiment, the Molecular Target comprises a small molecule drug target. In yet another embodiment, the Molecular Target comprises a nucleic acid. In yet further embodiment, the Molecular Target comprises polysaccharide or lipid.

In a specific embodiment, a plurality of (e.g., two or more) Molecular Targets are associated with a single identity nucleic acid; once a positive DART-Molecular Target is detected, a secondary round of screening can identity the particular Molecular Target out of the plurality which is responsible for the interaction.

DARTdance

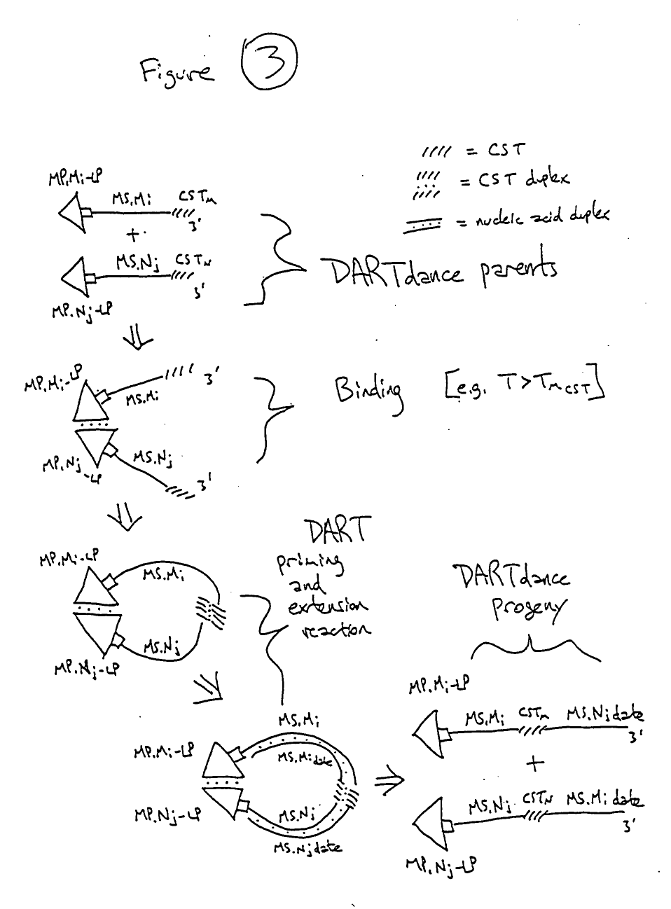

In another aspect, DARTdance provides another method of detecting a binding event between molecules that include DART.M and DART.N (see, e.g., FIG. 3). DARTdance uses the non-covalent association between DART components, for example the Molecular Points of two DARTs, to facilitate binding between Complementary Sequence Tail (CST) of the Molecular Shafts of the DARTs. A Complementary Sequence Tail (CST) can be a nucleic acid sequence at the 3′ terminus of a DART, DART.N, that is complementary to, and can bind specifically to, a nucleic acid sequence at the 3′ terminus of another DART, DART.M. The CST is typically not self-complementary and typically does not bind other DART components (e.g., MP, LP, or non-CST portions of MS). CST Duplex, including a double-stranded nucleic acid comprising two CSTs, results from pairing of the CST of one DART with a complementary CST of a second DART.

Once the DARTs bind to each other, the CSTs form a CST duplex. The CST duplex can then serve as a primer for nucleic acid synthesis, which generates the DARTdance progeny, typically bound to one another via a double-stranded nucleic acid duplex. These progeny contain nucleic acid sequence information derived from both DARTdance parent molecules. Like DARTex, DARTdance permits the simultaneous pair-wise analysis of two populations of DARTs (e.g., DART.N and DART.M populations). Generally, DARTdance progeny can be subjected to a variety of manipulations and analyzed by any of the methods described for DARTex.

In a typical embodiment, DARTdance is performed by combining non-identical DARTS (e.g., DART.M and DART.N species) or library of DARTs (e.g., DART.Mᵢ and DART.Nⱼ). The first DART or a library of DARTS can include a Molecular Point (MP.Mᵢ) covalently linked to a Linkage Polypeptide (LP2), which is covalently linked to a Molecular Shaft (MS.Mᵢ). The Molecular Shaft MS.Mᵢ typically includes sequences that encode the Molecular Point (MP.Mᵢ). The Molecular Shaft of DART Mi also includes a CST, CST_{M}. In this example, the elements of DART.Mᵢ are MP.Mᵢ-LP2-MS.Mᵢ-CST_{M}.

The second DART or library of DARTs can include a Molecular Point (MP.Nⱼ) covalently linked to a Linkage Polypeptide (LP1), which is covalently linked to a Molecular Shaft (MS.Nⱼ). The Molecular Shaft MS.Nⱼ typically includes sequences that encode the Molecular Point (MP.Nⱼ). The Molecular Shaft of DART Nⱼ also encodes CST, CST_{N}. In this example, the elements of DART.Nⱼ are MP.Nⱼ-LP2-MS.Nⱼ-CST_{N}. As will be also appreciated, it will often be useful to include primer annealing sites within MS.N and/or MS.M that permit subsequent PCR amplification, sequencing, restriction enzyme digestion, hybridization, cloning, or other manipulation of nucleic acids comprising these sequences, including but not limited to DARTs and DARTdance progeny.

CST_{M} and CST_{N} can hybridize to one another to form a CST duplex. The skilled artisan will appreciate that each of the Molecular Shafts DART.MᵢMS.Mᵢ and DART.N MS.Nⱼ can optionally include one or more primer annealing sites to facilitate subsequent amplification, sequencing, or other manipulations. MS.N and/or MS.M can also optionally include restriction endonuclease sites for restriction enzyme-mediated cleavage of the nucleic acid duplex produced by DARTdance or for other manipulation and/or analysis. MS.N and/or MS.M can also optionally include sequences to facilitate recombination, hybridization to targets, or other manipulations.

When DART Mᵢ and DART Nⱼ are contacted, the Molecular Points of DART.Mᵢ and DART.Nⱼ bind non-covalently or covalently. As a result, CST_{M} and CST_{N} can be brought into close proximity, which permits the CST of each Molecular Shaft to form a CST duplex. Each CST in the CST duplex can then serve as the primer for nucleic acid synthesis in the extension reaction. A polynucleotide polymerase can then be used to extend the duplex region. If RNA DARTs are used, reverse transcriptase or another suitable polymerase may be used. The nucleic acid polymerase can be covalently attached to one of the DARTS or can be added separately to the reaction. Following completion of polynucleotide synthesis, the resulting DARTdance progeny contain Molecular Shafts with nucleic acid sequence information derived from both interacting parent, DART.Nⱼ and DART.Mᵢ.

Referring to FIG. 3, the reciprocal exchange of nucleic acids that occurs during DARTdance results in the formation of two similar DARTdance progeny molecules. In one progeny molecule, MP.Mᵢ-LP2 is covalently linked to MS.Mᵢ and MS.Nⱼ′ in the following configuration: MP.Mᵢ-LP2-MS.Mᵢ-MS.Nⱼ′ (MS.Nⱼ′ is the complementary strand of MS.Nⱼ). In the other type of progeny, MP.Nⱼ-LP1 is covalently linked to MS.Nⱼ′ and MS.Mᵢ in the following configuration: MP.Nⱼ-LP1-MS.Nⱼ-MS.Mᵢ (MS.Mᵢ′ is the complementary strand of MS.Mi). These two molecules are non-covalently bound to one another at the completion of the extension reaction via a nucleic acid duplex between MS.Nⱼ-MS.Mᵢ′ and MS.Mᵢ-MS.Nⱼ′. The interactions between other DART components, for example MP.Mᵢ:MP.Nⱼ, can also contribute to association of the DARTs. The abundance and identity of DARTdance progeny molecules can be a function of the frequency of DART.Nⱼ:DART.Mᵢ binding events, the affinity of these DARTs for one another, the efficiency of DART priming the extension reactions, and the like.

DARTdance permits the detection of DART binding events, such as, for example, determining whether a single DART species, DART.N, interacts with another DART species, DART.M. In this example, two DARTdance progeny can be generated: MP.N-LP1-MS.N-MS.M′ and MP.M-LP2-MS.M-MS.N′. In another example, binding partners of a single DART.N species can be identified from library of DART.Mᵢ species. Possible DARTdance progeny include MP.Mᵢ-LP2-MS.Mᵢ-MS.N′ and MP.N-LP1-MS.N-MS.Mᵢ′.

In another example, the binding partners between libraries of DART species, for example, the binding partners between libraries of DART.Nⱼ species and DART.Mᵢ species, can be identified. Many different species of DARTdance progeny can result from this DARTdance reaction. For example, if MP.N₃-LP1-MS.N₃ interacts with MP.M₅-LP2-MS.M₅, the resulting progeny will include MP.M₅-LP2-MS.M₅-MS.N₃′ and MP.N₃-LP1-MS.N₃-MS.M₅′. If MP.N₈-LP1-MS.N₈ interacts with MP.M₁₂-LP2-MS.M₁₂, the resulting DARTdance progeny will include MP.N₈-LP1-MS.N₈-MS.M₁₂′ and MP.M₁₂-LP2-MS.M₁₂-MS.N₈′. The identities of the two DARTs, including their MP domains, whose interaction facilitated the DARTdance reaction can be revealed in the nucleic acid sequence contained in the DARTdance progeny. Generally speaking, these progeny will include MP.Mᵢ-LP2-MS.Mᵢ-MS.Nⱼ′ and MP.Nⱼ-LP1-MS.Nⱼ-MS.Mᵢ′.

In one embodiment, the initial interaction (or contacting) between the Molecular Points in a DARTdance binding reaction is performed under conditions where the CST duplex does not form (e.g., the temperature of the reaction (T) is greater than the melting temperature, Tₘ, for CST duplex formation). This initial binding reaction can be performed in vivo or in vitro in any suitable system in which DARTs are synthesized or to which they have been added and in which the formation of the CST duplex can be prevented without adversely affecting the purpose of the DARTdance assay.

Once the initial binding reaction is performed, DARTdance priming and extension occurs. This priming and extension reaction allows the formation of the CST duplex between DARTs bound in non-covalent complexes. Each CST in the CST duplex can serve as a primer for subsequent nucleic acid synthesis. This priming and extension reaction produces DARTdance progeny. The DARTdance priming and extension reaction can be performed in vitro or in vivo under conditions that allow CST duplex formation. It can be useful to purify DARTs and/or modify in vivo DART analysis systems to facilitate the DARTdance priming and extension reactions.

CST duplex formation is typically induced by reducing the temperature of the reaction below the Tₘ for the CST duplexes. CST duplex formation may be regulated by temperature, salt concentration, and the like. This can be useful in preventing CST duplex formation in the absence of a binding event between other components of the CST-containing DARTs. The activity of a polynucleotide polymerase (e.g., T4 DNA polymerase or TAQ DNA polymerase) can be stimulated in the reaction, by such means as, for example, the addition of the polymerase, the addition of nucleotides or other molecules required for polymerization or polymerase function and the like. Extension can also be performed by a nucleic acid polymerase already present in the reaction (e.g., an unpurified DNA polymerase in an intact or lysed bacterium or eukaryotic cell). The DART.N:DART.M complexes containing the CST duplex can be bound by the polymerase. The nucleic acid polymerase can catalyze the addition of nucleotides to the free 3′ termini of the Molecular Shafts (i.e., MS.N and MS.M). For example, the template sequence for the nucleic acids added by the polymerase to MS.N is MS.M, whereas the template for the nucleic acids added by the polymerase to MS.M is MS.N. At the completion of the extension reaction a DNA duplex is formed that includes the nucleic acid components of the DARTdance progeny MP.M-LP2-MS.M-MS.N′ and MP.N-LP1-MS.N-MS.M′.

The DART priming and extension reaction can be terminated, for example, under conditions where the CST duplex is dissociated (e.g., raising the temperature (T) to above the Tₘ for CST duplex formation), and/or inactivating the nucleic acid polymerase. As will be appreciated by the skilled artisan, in certain DARTdance applications it is desirable to induce conditions that do not cause the dissociation of the long nucleic acid duplexes generated during the extension reaction (e.g., if temperature shift is employed as above, the temperature (T) is lower than the Tₘ of the duplex of the DARTdance extension reaction products). Under other DARTdance applications it can be desirable to permit or stimulate the dissociation of the long nucleic acid duplexes generated during the extension reaction.

As will be appreciated, higher order DARTdance reactions involving two DARTs and one or more DARTs and/or non-DART molecules can also be performed. Similarly, DARTdance reactions can be performed to detect the interaction of a component of one DART species with a component of another DART species.

Once the DARTdance progeny are formed, the progeny can be prepared for subsequent analysis by a variety of methods, as described herein or as are known to the skilled artisan. Such methods include, but are not limited to, separation of the individual nucleic acid strands of the progeny. Such separation can be achieved, for example, by raising the temperature of the reaction above the melting temperature (Tₘ), by increasing the salt concentration, by the addition of organic reagents, and the like.

The DARTdance progeny can also be purified using methods described herein or as known to the skilled artisan. Such purification can be employed for several purposes, including but not limited to separating the nucleic acid from non-nucleic acid DART components, separating DARTdance progeny from other molecules, separating DARTs from other molecules, and the like. Restriction enzyme-mediated cleavage of the duplex DNA (e.g., MS.N-MS.M′:MS.M-MS.N′ duplex DNA) of DARTdance progeny can be performed using one or more restriction enzymes that are specific for double-stranded DNA. Such restriction digestion can be used to separate the nucleic acid molecule of the progeny (MS.N-MS.M′: MS.M-MS.N′ duplex) from the Linkage Polypeptides. The nucleic acids can also be separated from the DART progeny by the addition of postMS molecules, thereby triggering Linkage Polypeptide-mediated ligation of postMS to MS to release LP from nucleic acids, which can then be analyzed.

The nucleic acid portion of the DART progeny can also be amplified by polymerase chain reaction (PCR). Typically, reagents are added to DARTdance progeny for PCR amplification. These reagents can include, but are not limited to, a thermostable DNA polymerase (e.g., Thermus aquaticus DNA polymerase), primers that anneal to the primer annealing sites of MS.N and MS.M, nucleotides and other factors necessary for PCR. Thermal cycling can be performed according to standard PCR methodology. (See, e.g., U.S. Pat. Nos. 4,683,202, 4,683,195 and 4,800,159; Erlich (ed.), PCR Technology: Principles and Applications for DNA Amplification, Stockton Press, New York (1989); Innis et al. (eds.), PCR Protocols: A Guide to Methods and Applications, Academic Press, San Diego (1989); Mattila et al., 1991, Nucleic Acids Res. 19:4967-73; and Eckert and Kunkel, PCR Methods and Applications 1:17, Cold Spring Harbor Laboratory Press (1991); all of these references are incorporated by reference herein.) CST duplex formation is typically inhibited during PCR, such as by performing PCR above the Tₘ of CST duplex formation.

DARTdance progeny can be analyzed by any of numerous methods described below or by other methods known to the skilled artisan. For example, in cases where a library of DART.M species is assayed for binding partners in a library of DART.N species, DNA sequence analysis can be performed on the DARTdance progeny. In such cases, PCR amplification of DARTdance products can be useful to increase the amount of nucleic acid available for DNA sequence analysis. If DARTboard analysis is to be performed, it can be useful to remove one of the progeny pair because MS:MS hybridization can impair DARTboard formation or subsequent detection steps, as further described herein. One of the progeny can be removed by a variety of methods, including but not limited to, removing all DART.N molecules (e.g., using an antibody to immunoprecipitate all DARTs containing an LP). Such purification is typically performed under conditions that prevent DART.N:DART.M binding and CST duplex formation, and that limit MP:MP and MS:MS interactions and the like.

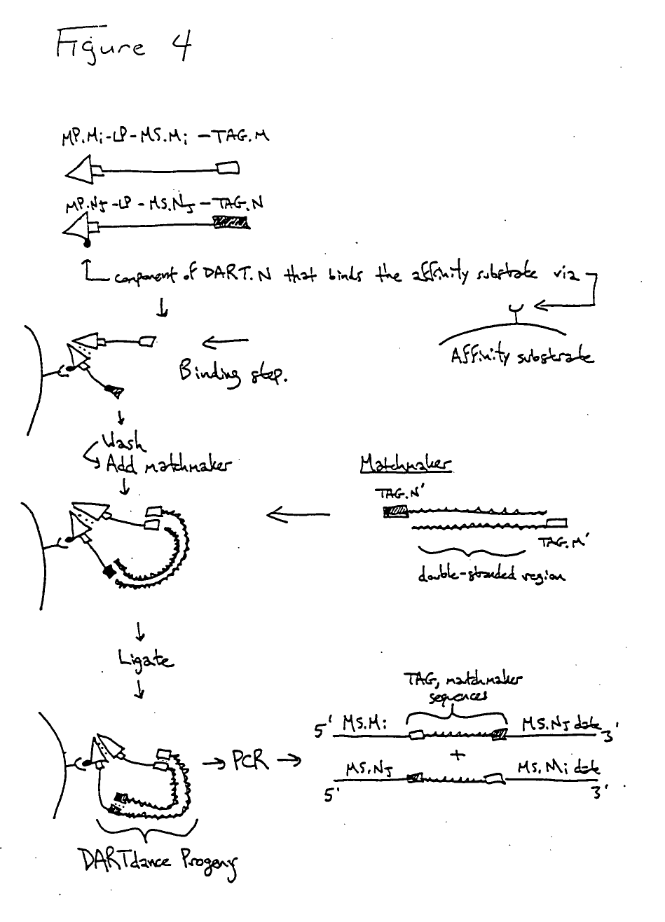

In another aspect of DARTdance, the 3′ termini of the DART.M and DART.N Molecular Shafts comprise TAG nucleic acid sequence. (See, e.g. FIG. 4.) A TAG sequences can be complementary to a portion of a Matchmaker nucleic acid. In such an aspect, the DART can optionally not include a CST. In another aspect of DARTdance, the 3′ termini of the DART.M and DART.N Molecular Shafts are hybridized to nucleic acids comprising TAG nucleic acid sequences. The TAG of DART.N is typically not complementary to the TAG or other portions of the Molecular Shaft of DART.M. Similarly, the TAG of DART.M is typically not complementary to the TAG or other portions of the Molecular Shaft of DART.N. Typically, each TAG is complementary to a different portion of a Matchmaker nucleic acid molecule.

In one embodiment, the Matchmaker molecule comprises nucleic acid sequences complementary to the DART.M TAG and nucleic acid sequences complementary to the DART.N TAG. In a typical embodiment, a Matchmaker is partially double-stranded and comprises the following nucleic acid sequences: 1—a single stranded sequence complementary to the DART.N TAG; 2—a single stranded sequence complementary to the DART.M TAG; and 3—a double stranded sequence through which the individual strands of the Matchmaker are hybridized to one another without interfering with their binding to TAGs. The Matchmaker can be prepared by any method known to the skilled artisan (e.g., by annealing two complementary oligonucleotides or digesting a nucleic acid molecule comprising the Matchmaker with one or more restriction enzymes). In a typical embodiment, the Matchmaker comprises sequences, such that when it is hybridized to both DART.M and DART.N TAG sequences, all portions of the Matchmaker are double-stranded. This TAG-Matchmaker-TAG complex can then be ligated, using methods known to the skilled artisan, to generate DARTdance progeny.

The modified DARTdance reaction is typically performed as described in the DARTdance example above with the following salient differences: 1—The binding reaction need not occur at a temperature above the CST melting point as DARTs employed in this embodiment do not have CSTs. 2—Typically, after the DARTdance binding reaction and any subsequent washes are complete, the Matchmaker is brought into contact with the DARTdance complexes. At this step one end of the Matchmaker can hybridize to the DART.N TAG and the other end of the Matchmaker can hybridize to the DART.M TAG. 3—The Matchmaker is ligated to the DART.N TAG and to the DART.M TAG with a suitable ligase, generating DARTdance progeny. These DARTdance progeny can be analyzed by the methods described below.

In another aspect, the DARTdance binding reaction is performed under conditions where one component of the DARTdance complex (e.g. DART.N or DART.M or another molecule bound to either DART.N or DART.M) is bound to an affinity substrate. As the skilled artisan will appreciate, the DARTdance complex bound to the affinity substrate can be washed to remove unbound molecules (e.g., DNA, RNA, proteins, peptides, small molecule drugs and the like), salts, buffers, detergents, and the like, or to add molecules, buffers, detergents and the like.

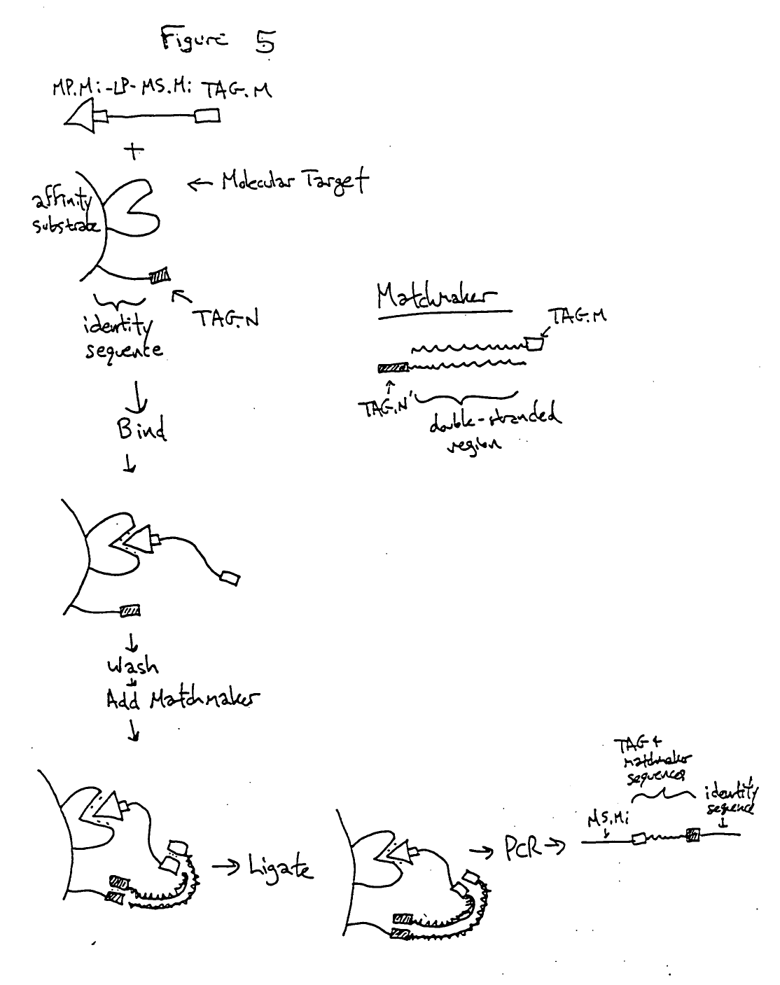

In another embodiment, DARTdance is performed with one DART (e.g., DART.M) and a substrate to which an Identity nucleic acid sequence and a Molecular Target are bound in close proximity (see, e.g., FIG. 5). The Identity nucleic acid typically comprises a TAG sequence and an Identity sequence. Each different Molecular Target can correspond to a different Identity sequence. The Identity sequence can be used to identify the Molecular Target following analysis of DARTdance progeny. Typically, the TAG sequence bound to the Identity sequence is complementary to a portion of the Matchmaker, and the TAG sequence of DART.M is complementary to a different portion of the Matchmaker, e.g., to single stranded overhangs of the Matchmaker at opposite ends of the molecule. The substrate and Matchmaker are designed to allow the Matchmaker to simultaneously hybridize to the TAG sequences of DART.M and the TAG sequences of the Identity nucleic acid when DART.M is bound to the Molecular Target corresponding to the Identity nucleic acid. Typically, the Matchmaker cannot simultaneously hybridize to the TAG sequences of DART.M and the TAG sequences of an Identity nucleic acid that does not correspond to the Molecular Target to which the DART is bound.

In one embodiment, different Molecular Targets can be bound to different agarose beads comprising the Identity nucleic acid and TAG corresponding to the Molecular Target. In another embodiment, the Molecular Target can comprise a polypeptide. In another embodiment, the Molecular Target comprises a small molecule drug. In another embodiment, the Molecular Target comprises a small molecule drug candidate. In yet another embodiment, the Molecular Target comprises a nucleic acid. In a further embodiment, the Molecular Target comprises a polysaccharide or lipid. In another embodiment of DARTdance, the 3′ termini of the DART.M and DART.N Molecular Shafts comprise TAG nucleic acid sequences and do not comprise CSTs. The TAG of DART.N is typically not complementary to the TAG or other portions of the Molecular Shaft of DART.M. Similarly, the TAG of DART.M is typically not complementary to the TAG or other portions of the Molecular Shaft of DART.N. Typically, each TAG is complementary to a portion of a Matchmaker nucleic molecule.

In another embodiment, the Matchmaker molecule comprises nucleic acid sequences complementary to the DART.M TAG and nucleic acid sequences complementary to the DART.N TAG. In a typical embodiment, as described above for DARTex, a Matchmaker is partially double-stranded and comprises the following nucleic acid sequences: 1-a single stranded sequence complementary to the DART.N TAG; 2—a single stranded sequence complementary to the DART.M TAG; and 3—a double stranded sequence through which the individual strands of the Matchmaker are hybridized to one another without interfering with their binding to TAGs. The Matchmaker may be prepared by any method known to the skilled artisan (e.g. annealing two complementary oligonucleotides or digesting a nucleic acid molecule comprising the Matchmaker with one or more restriction enzymes).

In another embodiment, different Molecular Targets can be bound to different agarose beads comprising an identity nucleic acid and tag combination that is typically unique to that Molecular Target. In another embodiment, the Molecular Target comprises a polypeptide. In another embodiment, the Molecular Target comprises a small molecule drug target, a nucleic acid, a polysaccharide or lipid.

In a specific embodiment, a plurality of (e.g., two or more) Molecular Targets are associated with a single Identity nucleic acid; once a positive DART-Molecular Target is detected, a secondary round of screening can identity the particular Molecular Target out of the plurality which is responsible for the interaction.

Analysis of DART Molecular Interaction Products

The progeny of DARTex and DARTdance molecular interaction assays are similar and can be analyzed by similar methods. Analysis of the progeny of both assays reveals the identities of the parent DARTs that participated in the DARTex or DARTdance reactions. Specifically, the progeny include DARTs with Molecular Shafts that contain nucleic acid information derived from both parent DARTs. Thus, the progeny of DARTex and DARTdance contain nucleic acid sequence information that reveals the identity of the interacting parent DARTs. There are a variety of ways of revealing this information embedded in the progeny molecules. A few exemplary methods are discussed below.

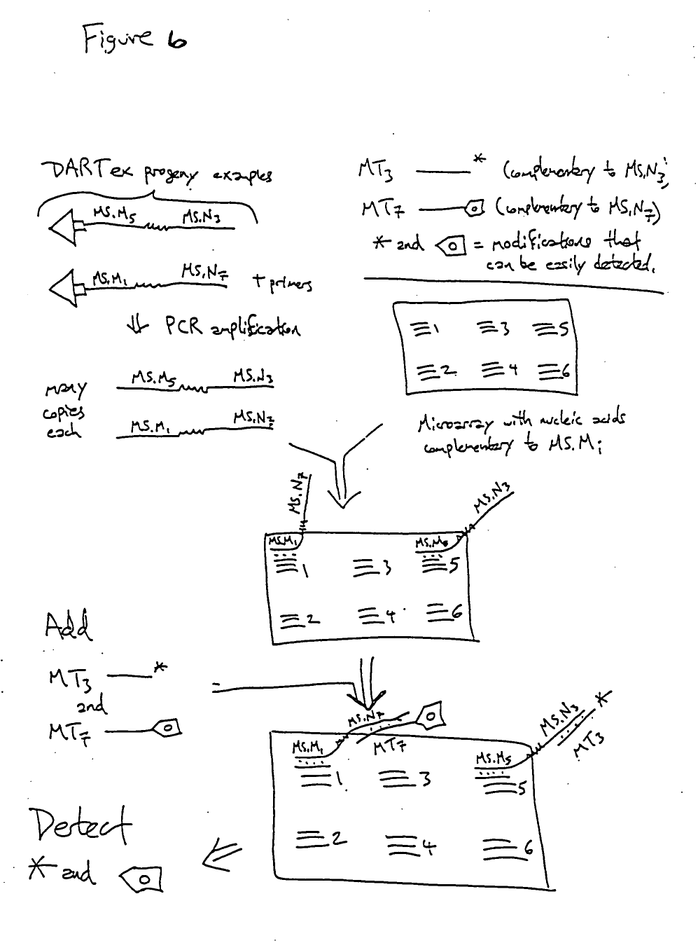

DART progeny can be analyzed by DNA sequence analysis to identify the nucleic acid sequences corresponding to the interacting Molecular Points, and the parent DARTs containing the interacting Molecular Points (see, e.g., FIG. 6). DNA sequence analysis can be performed by methods known in the art. (See, e.g., Maxam et al., 1980, Methods in Enzymology 65:499-560; Wallace et al., 1981, Gene 16:21-26; Ausubel et al., supra; Sambrook et al., supra.) The Molecular Shaft nucleic acid can be amplified by polymerase chain reaction using primers that anneal to primer binding sites on the DARTs. For example, to amplify DARTex progeny, one primer can be substantially identical to sequences near the 5′ end of MS.Mᵢ, and a second primer can be complementary to sequences near the 3′ end of MS.Nⱼ. Similarly, to amplify DARTdance progeny one primer can be substantially identical to sequences near the 5′ end of MS.Mᵢ, and the second primer can be substantially complementary to sequences near the 3′ end of MS.Nⱼ′. In some embodiments using RNA DARTs or DARTdance progeny containing RNA, the first round of synthesis can include reverse transcriptase to form a template DNA strand. The amplified Molecular Shaft sequences can be DNA sequenced, or cloned and then subjected to DNA sequence analysis.

Analysis of DART Molecular Interaction Products Using DARTBoards

DARTex and DARTdance reaction products (e.g., progeny) can be analyzed using DARTboard analysis (see, e.g., FIG. 5). DARTboard analysis is particularly suited for detection of different DART species or DARTex or DARTdance progeny. Generally, DARTboard analysis includes the following steps: An Affinity Substrate is prepared containing a plurality of different capture Molecular Targets. The DART progeny, or portions thereof, are contacted with the DARTboard, and the DART progeny bound to the capture Molecular Targets are detected directly, with probe Molecular Targets, or by other means. The DART progeny can optionally be labeled prior to contacting with the DARTboard Molecular Targets. As will be appreciated, it can often be useful to include primer annealing sites within MS.N and/or MS.M that facilitate PCR amplification, sequencing, restriction enzyme digestion, hybridization, cloning, or other manipulation of nucleic acids comprising these sequences, including but not limited to DARTs, DARTdance progeny, and DARTex progeny.

The capture Molecular Targets can be used to localize some or all of the DART progeny to defined sites on the Affinity Substrate. As will be appreciated by the skilled artisan, a variety of relationships can exist between the capture Molecular Targets of the Affinity Substrate and the DART progeny. For example, the capture Molecular Targets can be nucleic acid sequences complementary to a portion of MS.Mᵢ-CST-MS.Nⱼ′ (i.e., the Molecular Shaft of one of the DARTdance progeny) or a portion of MS.Mᵢ-RS1-MS.Nⱼ (i.e., the Molecular Shaft of one of the DARTex progeny). In one embodiment, the capture Molecular Targets specifically bind to the reaction product, MS.Mᵢ-CST-MS.Nⱼ′, but not to either parental Molecular Shaft (i.e., MS.Mᵢ or MS.Nⱼ). Alternatively, the Molecular Shafts can be complementary to a portion of the Molecular Shaft of one parental DART that is transferred by DART ligation to the other parental DART to form DART progeny). The Molecular Shafts can also be complementary to one of the parental Molecular Shafts. In a typical embodiment, the capture Molecular Targets on the array are not complementary to both MS.Nⱼ and to MS.Mᵢ. As will be appreciated, there are many nucleic acid capture Molecular Targets that can be used.

The DART progeny are typically contacted with the array under conditions suitable for nucleic acid hybridization of the DART progeny to the capture Molecular Targets. Such hybridization is typically conducted in an aqueous solution containing the DART progeny (e.g., DARTex and/or DARTdance progeny). Non-progeny molecules (e.g., parental DARTs) can optionally be separated from the DART progeny prior to hybridization. The DART progeny can also be purified prior to hybridization. In another embodiment, one of the DART progeny can be removed by, for example, immunoprecipitation using an antibody against one of the Molecular Points, or hybridization of the DART progeny with a nucleic acid that selectively binds to the DART progeny to be removed.

In some embodiments, the DART progeny can be preprocessed prior to contacting with the capture Molecular Targets. Such preprocessing can improve subsequent analysis of the DART progeny. For example, the Molecular Shafts of the DART progeny can be amplified by polymerase chain reaction. PCR amplification can be performed using primer-annealing sites in the Molecular Shafts. To amplify DARTex progeny, one primer can be identical to sequences near the 5′ end of MS.M and a second primer can be complementary to sequences near the 3′ end of MS.N. The resulting PCR amplification can selectively amplify DART progeny molecules containing both MS.M and MS.N. Similarly, to amplify DARTdance progeny, one primer can be identical to a sequence near the 5′ end of MS.Mᵢ, and the second primer can be identical to a sequence near the 5′ end of MS.Nⱼ. For RNA DARTex or DARTdance progeny containing RNA, the first round of synthesis can include a reverse transcriptase step to form a template DNA strand.

The hybridization of the DART progeny to the capture Molecular Targets is typically performed under hybridization conditions that minimize DART.M:DART.N interactions between the DARTs (i.e., the DART progeny are dissociated). For example, the hybridization can be performed under stringent conditions (e.g., relatively high salt and/or detergent concentration) that disrupt MP:MP, MP:LP, MP:MS, and/or LP:MS interactions. DART progeny molecules can also be treated with agents that destroy or inactivate the MP-LP component of the progeny molecules (e.g., with protease, heat, detergents, or EDTA) to reduce interference from non-nucleic acids in the analysis of DART progeny nucleic acids.

The DARTboard typically comprises MP.Mᵢ-LP2-MS.Mᵢ-CST-MS.Nⱼ′ DARTdance progeny or MP.Mᵢ-LP2-MS.Mᵢ-RS₁-MS.Nⱼ DARTex progeny bound to the corresponding Molecular Targets on the array (e.g., a probe complementary to MS.Mᵢ or MS.Nⱼ′ or MS.Nⱼ). The DARTboard can also include other DARTs and/or nucleic acids bound to the capture Molecular Targets on the array. Thus, the DARTboard can have no, one, or more than one DART progeny species bound to each discrete locus on the array. The DARTboard can optionally be washed to remove non-specifically bound molecules.

Following formation of the DARTboard, the bound DART progeny are detected. The DART progeny can contain a label capable of being detected when the DART is bound to the array. For example, the DART progeny can contain a label bound to the Molecular Point and/or Linkage Polypeptide of the DART. Alternatively, the DART progeny can be labeled during amplification, such as, for example, by use of a labeled primer or labeled nucleotides.

Alternatively, the DARTboard can be contacted with one or more probe Molecular Target (MT) molecules that bind to the DART progeny on the DARTboard. For example, the probe Molecular Target can be a nucleic acid complementary to a portion of the bound DART, a protein that binds to the DART (e.g., the Molecular Point, Linkage Polypeptide or Molecular Shaft), an antibody that binds to the Molecular Point or Linkage Polypeptide, another DART, or other molecules. The probe Molecular Target typically binds with specificity to a progeny DART or a complex containing one or more progeny DARTs. The probe Molecular Target typically does not bind to non-progeny molecules bound to the DARTboard. The probe Molecular Target can be one or more nucleic acid species, each complementary to a different MS.Mᵢ, or MS.Nⱼ, or MS.Nⱼ′ species. As will be appreciated by one skilled in the art, many other probe Molecular Target molecules will be useful and their selection depends on the composition of the DART parents and the expected DART progeny molecules. If the capture Molecular Targets on the array are complementary to MS.Nⱼ′, then the probe Molecular Target can be a nucleic acid molecule complementary to MS.Mᵢ. Similarly, if the capture Molecular Targets are complementary to MS.Mᵢ, then the probe Molecular Targets can be a nucleic acid molecules complementary to MS.Nⱼ′ in the case of DARTdance progeny or complementary to MS.Nⱼ in the case of DARTex progeny.

The probe Molecular Target is typically labeled. Such labels can include fluorescent molecule, a radionuclide, biotin, an affinity tag (e.g., a polyhistidine tract), and the like. One or more probe Molecular Targets can be used to detect DARTs bound to the DARTboard in a single detection. Such probe Molecular Targets are typically differentially labeled (e.g., different fluorescent dyes, such as green, red, yellow, and blue fluorescent dyes) so that the bound probe Molecular Targets can be distinguished. Prior to detecting, the DARTboard can be washed to remove non-specifically bound probe Molecular Targets.

The labeled probe Molecular Targets, or labeled DARTs, can be detected using a variety of methods known in the art. The detection protocol can vary, depending on the label used. For example, fluorescence emission/absorption spectra analysis can be employed to distinguish between probe Molecular Targets containing different fluorescent labels (e.g., green, red, yellow, and/or blue fluorescent dyes). By analogy, radiometric detection protocols can be employed to detect radionuclides. Immunological detection methods can be employed to detect, for example, biotin- or epitope-tagged probe Molecular Targets.

By analyzing the signals on the array, and the positions of the known loci on array, the identity of the DART progeny, and the parent DART(s), can be determined. For example, if DART MP.N₃-LP1-MS.N₃ interacts with DART MP.M₅-LP2-MS.M₅-RS₁ in a DARTex assay, the resulting DARTex progeny can include an MP.M₅-LP2-MS.M₅-RS₁-MS.N₃ DART. This DART progeny species can bind to a site on the DARTboard corresponding to (i.e., containing a nucleic acid complementary to) MS.N₃. The bound DART progeny species can bind to a labeled probe Molecular Target corresponding to (i.e., containing nucleic acid sequences complementary to) MS.M₅. The signal from the labeled probe Molecular Target can localize to the DARTboard locus that binds MS.N₃, thereby revealing that DART.N₃ (MP.N₃-LP1-MS.N₃) interacted with DART.M₅ (MP.M₅-LP2-MS.M₅) in the DARTex reaction. The skilled artisan will appreciate that many other DARTboard and probe Molecular Target compositions and combinations thereof may be successfully employed.

Similarly, in a DARTdance assay, if DART.N₃ (MP.N₃-LP1-MS.N₃-CST) interacts with DART.M₅(MP.M₅-LP2-MS.M₅-CST), the resulting DARTdance progeny can include DART MP.M₅-LP2-MS.M₅-CST-MS.N₃′ and MP.N₃-LP1-MS.N₃-CST-MS.M₅′. The latter DART progeny species will bind to a site on the DARTboard corresponding to (i.e., containing a nucleic acid complementary to) MS.N₃. This DART progeny species can bind to a labeled probe Molecular Target corresponding to (i.e., containing nucleic acid sequences complementary to) MS.M₅′. The signal from the labeled probe Molecular Target that binds MS.M5 can localize to the DARTboard locus that binds MS.N3, thereby revealing that DART.N₃ interacted with DART.M₅ in the DARTdance assay. The skilled artisan will appreciate that many other DARTboard and probe Molecular Target compositions and combinations thereof may be successfully employed.

DART Modulation of Gene Expression

In another aspect, DARTs can be used to modulate gene expression in vivo or in vitro. In particular, a component of a DART (e.g., a Molecular Point, a Linkage Polypeptide, and/or a Molecular Shaft) can be used to direct a DART to a defined molecular context, such as, for example, a unique site in a complex genome, to target specific molecules in cell or an in vitro reaction to a defined molecular context, or to drive the assembly of a multiple DART complex. For example, the Molecular Shaft and/or Molecular Points of DARTs can be used to bring different DARTs and/or non-DART molecules together in non-covalent complexes. These non-covalent complexes can have properties/activities distinct from the properties/activities of each individual molecule of the complex. Examples of the modulation of gene expression using DARTs are provided below, although the present invention is not intended to be limited by or to these examples.

The Molecular Shaft of the DARTs can be self-referential (i.e., containing a nucleic acid sequence encoding the Molecular Point) or non-self-referential (i.e., containing a nucleic acid sequences that do not encode the Molecular Point). In one embodiment, the Molecular Shaft can include nucleic acid sequences that will target a DART to a given gene sequence, thereby delivering its linked Molecular Point to that gene sequence as well. The proximity of the Molecular Point to the gene sequence can be used to modulate the expression of the gene. For example, DARTs can be used to control the abundance of an RNA species that hybridizes to the Molecular Shaft, such as, for example mRNA or viral RNA. The Molecular Point can be, for example, a protein that hydrolyzes RNA or DNA.

In one embodiment RNase DARTs are provided. RNase DARTs have a Molecular Point encoding a protein having RNase activity. Such a DART can be either an RNA DART or a DNA DART. RNA DARTS can be used, for example, when a limited lifespan for the DART is desired. The RNase DART can be expressed in a cell or delivered to a cell or in vitro reaction that contains the target RNA. For example, an RNA DART can be expressed in a cell, such as a human cell or a host cell, and will likely reside in close proximity to the protein synthetic machinery (e.g., ribosomes), which is where one class of RNA will be present. An RNase DART can also be targeted to the nucleus of a eukaryotic cell by including a nuclear localization sequence into the Molecular Point and/or Linkage Polypeptide.

Modulation of Gene Expression By RNase Antisense DARTs

In another embodiment, the DART is an RNase H antisense DART, that has RNase H as the Molecular Point, or a portion thereof. RNase H specifically cleaves double-stranded RNA, or RNA-DNA hybrids, but not single-stranded RNA. Thus, RNase H antisense DARTs can be used to selectively destroy a particular species of RNA in a complex mixture of molecules, either in vitro or in vivo. Such RNase H antisense DARTs can have Molecular Shafts of either RNA or DNA, or a mixture thereof. RNase H antisense DARTs having an RNA Molecular Shaft can have a limited lifespan.

An RNase H antisense DART has a Molecular Shaft, or a portion thereof, that is complementary to the target RNA. The DART can hybridize to target RNA (e.g., an mRNA species or a viral RNA species), and the RNase H activity will hydrolyze the target RNA. By administering such an RNA H antisense DART, the expression of a gene can be reduced or inhibited by destroying the mRNA of the gene.

Antisense RNase DART-mediated inhibition of gene expression can be utilized, for example, for the treatment inherited disorders with dominant-negative or gain-of-function pathogenetic mechanisms, for the suppression of oncogenes, or for the control of a variety of infectious agents. Such pathologic disorders include, for example, viral infections, inflammatory disorders, cardiovascular disease, cancers, genetic disorders and autoimmune diseases.

Antisense RNase H DARTs can also be used to inhibit the expression of a mutant protein or a dominantly active gene product, such as amyloid precursor protein that accumulates in Alzheimer's disease. Such DARTS can also be useful for the treatment of Huntington's disease, hereditary Parkinsonism, and other diseases. Antisense RNase H DARTs are also useful for the inhibition of expression of proteins associated with toxicity or gene products introduced into the cell, such as those introduced by an infectious agent (e.g., a retrovirus such as the human immunodeficiency virus).

Antisense RNase H DARTs whose Molecular Shafts are complementary to mRNAs encoding biological response modifiers can be used, for example, to reduce the expression levels of such biological response modifiers. Biological response modifiers include, for example, immunopotentiating agents such as cytokines, interleukins, interferons, tumor necrosis factor (TNF) and granulocyte-macrophage-colony stimulating factor (GM-CSF). Antisense RNAse H DARTs specific for biological response modifiers can be used to treat autoimmune (e.g., rheumatoid arthritis) and hyperimmune disorders (e.g., allergy). Antisense RNAse H DARTs can be delivered to a patient by a gene therapy vector, for example, as described infra.

DART Diagnostics

DARTs and DARTboards can be used to detect one or more species of non-DART molecules present in a complex mixture of molecules. For example, a disease-associated molecule (e.g., an antigen) for which an antibody or binding polypeptide has been defined can be detected using DART technology. Examples of the application of DART technology to DART diagnostics include the following:

In one assay, a collection of diagnostic DARTs can be used to screen a biological sample for disease-associated molecules (e.g., antigens). Such an assay can be performed by preparing one or more diagnostic DARTs in which the Molecular Point of the DARTs can specifically bind to disease-associated molecules present in relevant biological samples.

Suitable disease-associated molecules can include, for example, viral antigens, bacterial antigens, protozoal antigens, parasitic antigens, tumor-associated antigens, markers associated with a particular blood type, serotype, genetic disease, infectious disease or other biological systems. Thus in one embodiment of the present invention, diagnostic DARTs can be used to detect viral antigens, for example, antigens of hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus, polio virus, human immunodeficiency virus type I (HIV-I), and human immunodeficiency virus type II (HIV-II). In another embodiment, diagnostic DARTs can be used to detect bacterial antigens, for example antigens of mycobacteria rickettsia, mycoplasma, neisseria and legionella. In yet another embodiment, diagnostic DARTs can be used to detect parasitic antigens, for example antigens of chlamydia and rickettsia. In yet another embodiment, diagnostic DARTs can be used to detect parasitic antigens, for example antigens of leishmania, kokzidioa, and trypanosoma. In yet another embodiment, diagnostic DARTs can be used to detect tumor-associated antigens, for example KS ¼ pan-carcinoma antigen (Perez and Walker, 1990, J. Immunol. 142:3662-3667; Bumal, 1988, Hybridoma 7(4):407-415); ovarian carcinoma antigen (CA125) (Yu et al., 1991, Cancer Res. 51(2):468-475); prostatic acid phosphate (Tailer et al., 1990, Nucl. Acids Res. 18(16):4928); prostate specific antigen (Henttu and Vihko, 1989, Biochem. Biophys. Res. Comm. 160(2):903-910; Israeli et al., 1993, Cancer Res. 53:227-230); melanoma-associated antigen p97 (Estin et al., 1989, J. Natl. Cancer Inst. 81(6):445-446); melanoma antigen gp75 (Vijayasardahl et al., 1990, J. Exp. Med. 171(4):1375-1380); high molecular weight melanoma antigen (Natali et al., 1987, Cancer 59:55-63) and prostate specific membrane antigen. In yet other embodiments, diagnostic DARTs can be used to detect proteins whose amino acid substitution, is misfolding, mis-expression or overexpression is associated with a disease, such as prion proteins, proteins associated with neurodegenerative diseases, and the like. The skilled artisan will appreciate that many other such molecules or complexes of molecules can also be detected successfully using DART diagnostics.

The DARTs can optionally be at least partially purified. Such purification typically preserves the binding activities of the DART Molecular Points. The diagnostic DARTs can be contacted with a biological sample that may contain a disease-associated molecule. In parallel, the diagnostic DARTs can be contacted with positive and/or negative control samples (i.e., those containing or lacking known quantities of the relevant molecules). If the disease associated molecules are present in the biological sample, they can be bound by the corresponding diagnostic DARTs; similarly, disease associated molecules in the positive control can be bound by the corresponding diagnostic DARTs.

The diagnostic DART complexes can then be resolved. In one embodiment, the complexes are resolved in an array containing an ordered set of nucleic acid capture Molecular Targets. Each nucleic acid capture Molecular Target is complementary to a sequence present in the diagnostic DARTs. Typically, each diagnostic DART can bind to nucleic acid capture Molecular Targets at a separate locus on the array. When the DARTs bind to the corresponding capture Molecular Targets, a DARTboard is formed.

In a specific embodiment, the steps for the resolution of diagnostic DARTs (and their bound disease-associated molecules) on a DARTboard can include, for example: (a) diluting the biological sample harboring DART-antigen complexes in blocking reagent to form a DART-sample mixture; (b) incubating the array with separate blocking reagent; (c) contacting the DART-sample mixture with the array to allow each diagnostic DART species to bind its corresponding capture Molecular Target on the array; (d) washing the diagnostic DARTboard with excess blocking buffer to remove unbound material; and (e) detecting the presence or absence of the disease-associated molecules (e.g., antigens) on the DARTboard.

In another embodiment, the assembled DARTboard comprising diagnostic DARTs not yet exposed to the biological sample can be contacted with biological sample according to, for example, the following steps (a) diluting the mixture harboring diagnostic DARTs in blocking reagent to form a DART mixture; (b) incubating the array with blocking reagent; (c) contacting the DART mixture with the array to allow each diagnostic DART species to bind its corresponding capture Molecular Target on the array; (d) washing the diagnostic DARTboard with excess blocking buffer to remove unbound material; optionally washing in other buffer conditions as well; (e) contacting the diagnostic DARTboard with the biological sample, optionally mixed with blocking reagent, and (f) detecting the presence or absence of the disease-associated molecules (e.g., antigens) on the DARTboard.

Disease-associated molecules (e.g., antigens) bound to diagnostic DARTs can be detected using a variety of methods, including, but not limited to, direct detection using analytical chemical methods (e.g., mass spectroscopic analysis of particular DARTboard loci); and/or immuno-chemical methods (e.g., using antibodies directed against the disease-associated molecules). Using these methods or similar methods, molecules bound to DARTs on the DARTboard can be identified. In some embodiments, the amount of the molecule bound to the diagnostic DART can be quantified.

In another embodiment, diagnostic DART assays can be performed in duplicate, in triplicate, and the like. For example, diagnostic DART assays can be performed in triplicate using three identical arrays. Each array is a substrate having nucleic acid capture Molecular Targets at specified loci. One array is contacted with a DART+ experimental sample mixture (e.g., collection of diagnostic DARTs and biological sample). In parallel, the other arrays are contacted with positive and negative control mixtures containing or lacking, respectively, known quantities of the relevant disease-associated molecules. The presence of the disease-associated molecules on the resulting DARTboard can be detected and quantified as described above.

DART diagnostics can allow one or more biological samples to be simultaneously analyzed for the presence of many different disease-associated molecules. The amounts of the different disease-associated molecules can optionally be quantified. If the interaction of the Molecular Point and the disease-associated molecules is covalent (e.g., by using activated antibodies), then the DARTs can be stored for prolonged periods of time prior to analysis and quantitation on a DARTboard. Such storage can be performed because the disease-associated molecules can be denatured without affecting DARTboard formation. Therefore, a collection of diagnostic DARTs can be shipped as an analytical reagent, contacted with a biological sample (e.g., serum from an individual infected with a disease), and then shipped to a laboratory for DARTboard assembly and subsequent detection of the bound molecules. Such procedures optionally can be performed without the need to keep the DART-complexes cold or to require rapid transport of the diagnostic DARTs and/or the DART-molecule complexes. This benefit can be important for diagnostic technology applications in situations where refrigeration is impractical, expensive, sporadic, and/or unreliable.

DART Probes

DART probes can be used to detect Molecular Targets (MTs) in vitro, in vivo, and in situ in tissue samples. The Molecular Targets can include nucleic acid molecules (e.g., a particular mRNA species), protein molecules (e.g., a particular G-protein coupled receptor), protein modifications (e.g., phosphotyrosine residues on polypeptides), small molecules (e.g., hormones), or other molecules. The development and use of DART probes that bind known and novel Molecular Targets can be used, for example, to unveil and decode the information embedded in biological and experimental systems.

DART Probe Compositions

DARTs can be used as molecular probes to detect a wide range of Molecular Targets in a variety of in vivo, in vitro, and in situ assays. Generally, DART probes can contain sequences for specific Molecular Target recognition and binding sequences for detection.

Molecular Target binding can be mediated by the Molecular Point, the Linkage Polypeptide and/or the Molecular Shaft of one or more DARTs. For example, the Molecular Shaft can be complementary to a target mRNA species. In another example, the Molecular Point can include a domain that binds a particular protein target.

DART probes, and the binding of DART probes to Molecular Targets, can be detected by a label attached to the Molecular Point, the Linkage Polypeptide and/or the Molecular Shaft of one or more DARTs.

In one embodiment, the Molecular Shaft of a DART probe is labeled, for example with a fluorescent or radiolabeled tag. Molecular Shaft labeling can be achieved by incorporation of fluorescent or radiolabeled nucleotides during Molecular Shaft synthesis. The wide range of labeled nucleotides that can be successfully employed will be appreciated by the skilled artisan and include, but are not limited to, deoxynucleoside triphosphates, dideoxynucleoside triphosphates labeled with ³²P, ³³P, ³⁵S, fluorescein, digoxigenin, biotin, Cy5, Cy3, and rhodamine. Commercially available labeled nucleotides include but are not limited to ³²P-dATP, ³³P-dATP, ³⁵S-dATP, and fluorescein-15-dATP.

In another embodiment, the labeled portion of a DART probe is the Molecular Point. The Molecular Point can comprise a fluorescent protein, a bioluminescent protein, or a chemiluminescent protein, or green fluorescent protein (GFP), and the like. In a most typical embodiment, the Molecular Point comprises a fluorescent molecule, e.g., firefly luciferase. In a typical mode of the embodiment, the Molecular Point comprises GFP from Aequorea victoria or a mutant thereof. GFP can be encoded by its naturally-occurring coding sequence or by a coding sequence that has been modified for optimal human codon usage (see, e.g., U.S. Pat. No. 5,874,304) when the Molecular Point is generated in a mammalian cell line. Mutations can be introduced into the coding sequence to produce GFP mutants with altered fluorescence wavelength or intensity or both. Such mutations are largely in the vicinity of residues 65-67, which form the chromophore of the protein. Examples of useful GFP mutations for incorporation into the DART probes of the present invention can be found in U.S. Pat. Nos. 5,777,079 and 5,804,387 and International Publication WO97/11094. In another typical mode of the embodiment, the GFP mutant is a blue GFP. Examples of blue GFPs are described by Heim and Tsien (1996, Curr. Biol. 6:178-82). In yet another typical mode of the embodiment, the Molecular Point comprises a yellow or-red-orange fluorescent proteins (Matz et al., 1999, Nature Biotechnol. 17:969-973).

As will be appreciated by the skilled artisan, a wide variety of different DART probe compositions can be prepared. Suitable examples include those in the following Table 2:

TABLE 2

| MP | MS | Uses |
|---|---|---|
| GFP | complementary to | in situ GFP labeling of target |
| | target nucleic acid | nucleic acid |
| HRP | complementary to | in situ HRP labeling of target |
| | target nucleic acid | nucleic acid |
| βgluc | complementary to | in situ βgluc labeling of target |
| | target nucleic acid | nucleic acid |
| βgal | complementary to | in situ βgal labeling of target |
| | target nucleic acid | nucleic acid |
| Protein tag | complementary to | in situ protein tag labeling of target |
| | target nucleic acid | nucleic acid |
| Peptide tag | complementary to | in situ peptide tag labeling of target |
| | target nucleic acid | nucleic acid |
| Antibody | short sequence | in situ Cy3 labeling of target |
| | labeled with Cy3 | molecule |
| Antibody | short sequence | in situ Cy5 labeling of target |
| | labeled with Cy5 | molecule |
| Antibody fused | many possible MSs | in situ GFP labeling of target |
| to GFP | | molecule |
| Antibody fused | many possible MSs | in situ HRP labeling of target |
| to HRP | | molecule |
| Antibody fused | many possible MSs | in situ βgluc labeling of target |
| to βgluc | | molecule |
| Antibody fused | many possible MSs | in situ βgal labeling of target |
| to βgal | molecule | |

DART Probe Uses

DART probes can be used in a wide variety of assays including, but not limited to, in vivo assays, in vitro assays, and in situ assays. Examples of DART probe uses are briefly described below. As will be appreciated, many other uses are possible and within the scope of the present invention.

In vivo, DARTs comprising a Linkage Polypeptide, a Molecular Shaft, and a Molecular Point can be used to detect the abundance of Molecular Target molecules, such as RNA or DNA. In one embodiment, the Molecular Target is an mRNA species, and two DARTs are co-expressed in the cell where the abundance of the Molecular Target is to be determined. These two DARTs comprise Molecular Shafts that are not substantially complementary to one another but are each complementary to different sequences within the Molecular Target, such that a ternary complex can be formed between the Molecular Target and the two DARTs. The binding between each of these molecules is facilitated by nucleic acid duplex formation between the Molecular Target and the Molecular Shaft of each DART. The Molecular Points of these DARTs can associate, and this association is facilitated by, stabilized by, and typically depends upon the formation of the ternary complex described above. This association results in a detectable signal. This signal can comprise a fluorescent output (e.g. the two molecular points can be labeled or detected using fluorescence resonance energy transfer (FRET) tools), a biochemical output (e.g. the assembly of beta-galactosidase or another enzymatic activity), and the like. In this example, two DARTs act in concert as probes to monitor the abundance of a Molecular Target in vivo.

In vitro, a DART having an LP, a MS complementary to a Human Immunodeficiency Virus RNA sequence, and a MP containing GFP can be used to screen patient samples for the presence of HIV RNA.

In situ, a DART having a Molecular Shaft complementary to a portion of mRNA species X, an LP, and an MP containing HRP can be used to monitor the abundance and localization of X mRNAs in parallel in situ hybridization studies of diseased and healthy tissue samples.

Single-Step Detection Using DART Probes

DART probes can be used as single-step detection probes, where a label is linked to the DART, as illustrated by the examples above. This can bypass the requirement for multiple rounds of hybridization and/or binding events that increase the time and cost of each assay.

Multiple Step Detection Using DART Probes

Under certain circumstances, particularly where the abundance of the Molecular Target is low and/or the specific activity of detection reagents is low, it can be useful to include an amplification step into the detection protocol. DART probes can be used to amplify signals during molecular detection events. This can be achieved, for example, by exploiting the modular assembly properties of DARTs (infra). For example, if a primary DART whose Molecular Point binds a Molecular Target contains a Molecular Shaft with multiple copies of a repeated element, then a secondary DART containing a label (e.g., HRP) and a Molecular Shaft with sequences complementary to one of the repeated elements can be used to amplify the detection signal. Multiple secondary DARTs can bind to each primary DART, thereby amplifying the signal associated with each Molecular Target binding event.

Additional Uses of DART Probes

Generally, DART probes can be used in any context, in vitro, in vivo, and/or in situ, where a Molecular Target binding event occurs and DARTs can be delivered or synthesized. Examples include but are not limited to DNA or protein arrays, Western analysis, Northern analysis, Southern analysis, in vivo in live cells, in clinical samples, in situ in tissue samples, and in other systems. DARTs can be used in a variety of settings to deliver non-nucleic acid labels (e.g., GFP, HRP, lacZ, and the like) to nucleic acid Molecular Targets (e.g., mRNAs complementary to the Molecular Shaft) and nucleic acids and nucleic acid labeling methods (e.g., Cy3, Cy5, and the like) to non-nucleic acid Molecular Targets (e.g., proteins). DART probes can be used to bind and detect Molecular Targets in vitro, in vivo, and in situ in tissue samples. The development and use of DART probes that bind existing and novel Molecular Target can be used to unveil and decode the information embedded in biological, clinical, experimental, and other samples.

Directed Molecular Evolution

In another aspect, DARTs can be employed for directed molecular evolution, termed DarwinDART, applications. Typically, such directed evolution begins with a lead molecule possessing features that can be enhanced, extended or modified. The lead molecule is evolved by repeatedly cycling it through three processes: mutagenesis, selection and amplification of the selected molecules. The resulting molecules are then used as ‘lead’ compounds for subsequent rounds of mutagenesis, selection, and amplification. By executing multiple cycles of these processes, molecules with specific activities or functions can be generated. The selected molecules can also be unambiguously identified (e.g., by sequencing the nucleic acid associated with them).

The DarwinDART aspect is directed to methods for employing DARTs to generate a selected polynucleotide sequence or population of selected polynucleotide sequences, typically in the form of amplified and/or cloned polynucleotides, whereby the selected polynucleotide sequence(s) possess a desired phenotypic characteristic (e.g., encoding a polypeptide, promote transcription of linked polynucleotides, binding a protein, and the like) which can be selected. One method of identifying polypeptides that possess a desired structure or functional property, such as binding to a predetermined biological macromolecule (e.g., a receptor), involves the screening of a large library of polypeptides for individual library members which possess the desired structure or functional property conferred by the amino acid sequence of the polypeptide.

A significant advantage of the DarwinDART methods is that no prior information regarding an expected ligand structure is required to isolate peptide ligands or antibodies of interest. The peptide identified can have biological activity, which is meant to include at least specific binding affinity for a selected receptor molecule and, in some instances, can further include the ability to block the binding of other compounds, to stimulate or inhibit metabolic pathways, to act as a signal or messenger, to stimulate or inhibit cellular activity, and the like.

DarwinDART can be performed in a variety of different ways, either in vitro or in vivo. DarwinDART typically uses self-referential DARTs. A self-referential DART is one in which the Molecular Shaft includes a nucleic acid sequence that encodes at least a portion of the Molecular Point to which it is covalently linked. Self-referential DARTs can be used because the Molecular Shaft sequence provides information about the identity of the corresponding Molecular Point.

DarwinDART can be used to alter the binding affinity of a particular polypeptide for a given Molecular Target (MT). The Molecular Target can be, for example, a protein, receptor, polysaccharide, small organic molecule, lipid, carbohydrate and the like. Nucleic acids harboring sequences encoding a lead polypeptide can be mutagenized, creating a large and diverse library of sequences encoding lead polypeptide variants. Such mutagenesis can be performed by, for example, PCR-directed mutagenesis, chemical mutagenesis in E. coli, or other in vivo or in vitro methods known in the art. (See, e.g., Sambrook et al., supra.) The mutagenized nucleic acids can then be used to form the Molecular Points of DNA or RNA DART. For example, the mutagenized nucleic acids can be cloned into expression constructs. The Molecular Shaft sequences of these constructs typically include PCR primer annealing sites, and at least one cloning site (e.g., a restriction endonuclease site), flanking the Molecular Point coding regions. These sites can be used to PCR amplify the sequences encoding the Molecular Point, and to clone the resulting PCR product back into the expression construct. Alternatively, the DART expression constructs can be mutagenized directly.

In an aspect of the invention, mutator strains of host cells are used to enhance recombination of more highly mismatched sequence-related polynucleotides. Bacterial strains such as MutL, MutS, MutT, or MutH or other cells expressing the Mut proteins (e.g., XL-1 red; Stratagene, San Diego, Calif.) can be used as host cells for shuffling of sequence-related polynucleotides by in vivo recombination. Other mutation-prone host cell types can also be used, such as those having a proofreading-defective polymerase (see, e.g., Foster et al. (1995) Proc. Natl. Acad. Sci. (USA) 92:7951, incorporated herein by reference). Mutator strains of yeast can be used, as can hypermutational mammalian cells, including ataxia telangiectasia cells, such as described in Luo et al. (1996, J. Biol. Chem. 271:4497, incorporated herein by reference).

The expression constructs containing the mutagenized nucleic acids can be used to form libraries of DART molecules, either in vivo or in vitro (supra). The expressed DARTs can optionally be isolated and at least partially purified from other cellular components using DART purification schemes (See supra). For in vivo selection, the mutagenized DART library is typically expressed in the host cell type of interest.

The selection step of the molecular evolution scheme can generally be divided into three parts: DART binding to the Molecular Target(s) (MT) in vitro and/or in vivo; separating the MT-bound DARTs that comprise the selected DARTs from non-specifically MT-bound DARTs and DARTs not bound to MT; and eluting the selected DARTs from the Molecular Targets. Each of these steps is described in greater detail below.

The library of mutagenized DARTs can be contacted (bound) to the Molecular Targets in vitro or in vivo. For in vitro binding, DART libraries are typically contacted with the Molecular Targets under conditions suitable for binding of at least some of the DARTs to the Molecular Targets. The Molecular Targets are typically immobilized on a substrate. Suitable substrates include, for example, glass slides, silicon wafers, the wells of a microtiter plate, nitrocellulose membranes, agarose beads, polystyrene beads, magnetic beads, or any other solid or semi-solid surfaces on which Molecular Targets can be attached or immobilized. These Molecular Targets can be attached or immobilized to the substrate before or after DART binding, according to the contacting protocol.

DART binding to Molecular Targets can also occur in vivo. For such in vivo applications, the Molecular Targets typically bind to DARTs, and can also serve as Affinity Substrates for the purification of DART:MT (or optionally DART-MT) complexes. Further, cells containing DARTs can be screened for desired phenotypes or features. After transformation, the host cell transformants can be placed under selection to identify those host cell transformants which contain mutated specific nucleic acid sequences having the qualities desired. For example, if increased resistance to a particular drug is desired, the transformed host cells can be subjected to increased concentrations of the particular drug and those transformants producing mutated proteins able to confer increased drug resistance can be selected. If the enhanced ability of a particular protein to bind to a receptor is desired, then expression of the protein can be induced from the transformants and the resulting protein assayed in a ligand binding assay by methods known in the art to identify that subset of the mutated population which shows enhanced binding to the ligand. Alternatively, the protein can be expressed in another system to ensure proper processing.

Once a subset of the first recombined specific nucleic acid sequences (daughter sequences) having the desired characteristics are identified, they can then be subjected to a second round of mutagenesis, amplification, and selection, as desired.

The host cells can then be clonally replicated and selected for the marker gene present on the vector. Only those cells having the vector will grow under the selection.

The host cells which contain a vector can then be tested for the presence of favorable mutations. Such testing can include placing the cells under selective pressure, for example, if the gene to be selected is an improved drug resistance gene. If the vector allows expression of the protein encoded by the mutated nucleic acid sequence, then such selection can include allowing expression of the protein so encoded, isolation of the protein-and testing of the protein to determine whether, for example, it binds with increased efficiency to the ligand of interest.

To facilitate purification of the DART/MT complexes, the Molecular Targets can have an affinity tag (e.g., 6×HIS, FLAG, and the like). In some embodiments, the Molecular Target and the mutagenized DART library can be co-expressed in host cells. During this co-expression, the Molecular Points of the DARTs can bind to Molecular Targets, forming DART/MT complexes. The DART/MT complexes can then be co-purified, and immobilized on a supported substrate. Affinity purification using immobilized reagents directed against the Molecular Target affinity tag (anti-FLAG antibodies immobilized on agarose beads) can be used for the co-purification step.

Immobilized DART-MT complexes can optionally be washed to remove DART and/or other molecules that are not specifically bound to the Molecular Target. The stringency of the wash conditions can be varied to enrich for those molecules that display tighter binding. The selected DARTs can then be eluted from the DART/MT complexes, by for example, heating, or incubating the complexes in the presence of excess soluble Molccular Target, high salt, high or low pH, or other means previously determined to disrupt the DART:MT interactions and to cause the DARTs to dissociate from the immobilized Molecular Targets.

The Molecular Shafts, which typically contain sequences encoding the Molecular Point, of selected DARTs are typically amplified using appropriately chosen PCR primers. The amplified nucleic acids then provide nucleic sequences coding for the next generation of lead compounds. These lead compounds can then be processed through additional cycles of mutagenesis, selection, and amplification. The cycles can be repeated until a single DART species, or a suitable number (e.g., a few) DART species, are identified that encode a Molecular Point with the desired affinity for the Molecular Target. The nucleic acids of the selected DART(s) optionally can be cloned and/or sequenced to provide information about the identity of the corresponding selected Molecular Point.

As will be appreciated by the skilled artisan, using the methods described herein or known to the skilled artisan, DarwinDART can also be used to select nucleic acids that bind to Molecular Targets, as well as Linkage Polypeptide variants with desired features, DARTs whose presence results in an altered phenotype of the cell in which they reside and the like.

DarwinDART can be used to identify molecular reagents for the treatment of specific diseases or conditions, for diagnostic applications, for research applications, and for other commercially valuable purposes. For example, proteins or nucleic acid can be evolved to bind, with high specificity, to targets present on the surface of cancerous, diseased, or infected cells. Such ‘evolved’ molecules can then be used to direct therapeutic medicines specifically to the relevant biological targets, or used in diagnostic applications to detect the presence of targets. In addition, antibody-like molecules can be ‘evolved’ to act as sensitive and specific tools in a variety of molecular diagnostics applications. These applications include not only the detection of molecular markers for inherited, acquired, and communicable diseases, but also, the monitoring of pharmaceutics and chemotherapeutics present in biological samples derived from clinical patients. Further, proteins and nucleic acids can be evolved to efficiently execute enzymatic reactions in harsh environments where the lead molecule cannot function. These environments are commonly found in industrial and manufacturing settings.

Like bacteriophage display and other directed molecular evolution technologies, DarwinDART can be advantageous in the discovery and development of so-called single-chain fragment variable (scFv) libraries (see, e.g., Marks et al., 1992, Biotechnology 10:779; Winter and Milstein, 1991, Nature 349:293:Clackson et al., 1991, Nature 352(6336):624-8; Marks et al., 1991, J. Mol. Biol. 222:581; Chaudhary et al., 1990, Proc. Natl. Acad. Sci. USA 87:1066; Chiswell et al., 1992, TIBTECH 10:80; McCafferty et al., 1990, Nature 348(6301):552-4; and Huston et al., 1988, Proc. Natl. Acad. Sci. USA 85:5879). Various embodiments of scFv libraries displayed on bacteriophage coat proteins have been described.

Single-chain analogues of Fv fragments and their fusion proteins have been reliably generated by antibody engineering methods. The first step generally involves obtaining the genes encoding V_{H} and V_{L} domains with desired binding properties; these V genes can be isolated from a specific hybridoma cell line, selected from a combinatorial V-gene library, or made by V gene synthesis. The single-chain Fv is formed by connecting the component V genes with an oligonucleotide that encodes an appropriately designed linker peptide, such as (Gly-Gly-Gly-Gly-Ser)₃ (SEQ. ID NO: 32) or equivalent linker peptides. The linker bridges the C-terminus of the first V region and N-terminus of the second, ordered as either V_{H}-linker-V_{L} or V_{L}-linker-V_{H}. In principle, the scFv binding site can faithfully replicate both the affinity and specificity of its parent antibody combining site.

The linked polynucleotide of a library member provides the basis for replication of the library member after a screening or selection procedure, and also provides the basis for the determination, by nucleotide sequencing, of the identity of the displayed peptide sequence or V_{H} and V_{L} amino acid sequence. The displayed peptide(s) or single-chain antibody (e.g., scFv) and/or its V_{H} and V_{L} domains or their CDRs can be cloned and expressed in a suitable expression system. Often polynucleotides encoding the isolated V_{H} and V_{L} domains will be ligated to polynucleotides encoding constant regions (C_{H} and C_{L}) to form polynucleotides encoding complete antibodies (e.g., chimeric or fully-human), antibody fragments, and the like. Often polynucleotides encoding the isolated CDRs will be grafted into polynucleotides encoding a suitable variable region framework (and optionally constant regions) to form polynucleotides encoding complete antibodies (e.g., humanized or fully-human), antibody fragments, and the like. Antibodies can be used to isolate preparative quantities of the antigen by immunoaffinity chromatography. Various other uses of such antibodies can be to diagnose and/or stage disease (e.g., neoplasia), and for therapeutic application to treat disease, such as for example: neoplasia, autoimmune disease, AIDS, cardiovascular disease, infections, and the like.

Bacteriophage display of scFv has already yielded a variety of useful antibodies and antibody fusion proteins. A bispecific single chain antibody has been shown to mediate efficient tumor cell lysis (see, e.g., Gruber et al., 1994, J. Immunol. 152:5368). Intracellular expression of an anti-Rev scFv has been shown to inhibit HIV-1 virus replication in vitro (see, e.g., Duan et al., 1994, Proc. Natl. Acad. Sci. USA 91:5075), and intracellular expression of an anti-p21^{ras} scFv has been shown to inhibit meiotic maturation of Xenopus oocytes (see, e.g., Biocca et al., 1993, Biochem. Biophvs. Res. Commun. 197:422. Recombinant scFv which can be used to diagnose HIV infection have also been reported, demonstrating the diagnostic utility of scFv (see, e.g., Lilley et al., 1994, J. Immunol. Meth. 171:211). Fusion proteins wherein an scFv is linked to a second polypeptide, such as a toxin or fibrinolytic activator protein, have also been reported (see, e.g., Holvost et al., 1992, Eur. J. Biochem. 210:945; Nicholls et al., 1993, J. Biol. Chem. 268:5302).

DARTs that encode a variable segment peptide sequence of interest or a single-chain antibody of interest are selected from the library by an affinity enrichment technique. This can be accomplished by means of an immobilized macromolecule or epitope specific for the peptide sequence of interest, such as a receptor, other macromolecule, or other epitope species. Repeating the affinity selection procedure provides an enrichment of library members encoding the desired sequences, which can then be isolated for amplification, mutagenesis, sequencing, and/or for further propagation and affinity enrichment.

DARTs without the desired specificity are removed by washing. The degree and stringency of washing required will be determined for each peptide sequence or single-chain antibody of interest and the immobilized predetermined macromolecule or epitope. A certain degree of control can be exerted over the binding characteristics of the nascent peptide/DNA complexes recovered by adjusting the conditions of the binding incubation and the subsequent washing. The temperature, pH, ionic strength, divalent cations concentration, and the volume and duration of the washing can select for nascent peptide/DNA complexes within particular ranges of affinity for the immobilized macromolecule. Selection based on slow dissociation rate, which can be predictive of high affinity, is often the most practical route. This can be done either by continued incubation in the presence of a saturating amount of free predetermined macromolecule, or by increasing the volume, number, and length of the washes. In each case, the rebinding of dissociated nascent peptide/DNA or peptide/RNA complex is prevented, and with increasing time, nascent peptide/DNA or peptide/RNA complexes of higher and higher affinity are recovered.

The preceding references and discussion serve to underscore the utility of the present DarwinDART invention in identifying novel molecules with value in therapeutics, pharmaceutics, diagnostics, research applications and the like. The skilled artisan will thus appreciate that DarwinDART has significant advantages in the directed molecular evolution area.

Modular DART Assembly (MDA)

In another aspect, compositions and methods for modular DART assembly (MDA) are provided. A modular DART assembly is a complex comprising two or more DARTs and optionally comprising additional polypeptides or other molecules. These modular DART assemblies can possess features and/or activities not exhibited by their individual components.

The following description exemplifies modular DART assembly and its uses, including methods of forming MDAs in vivo and in vitro (DART/DART interactions (e.g., MP:MP and MS:MS interactions)), in vivo and in vitro applications of MDAs, and methods for controlling MDA assembly, disassembly, and activity.

Modular DART Assembly (MDA) Composition

Modular DART assemblies comprise two DARTs and can optionally comprise additional DART and/or non-DART molecules. Each intermolecular interaction or contact between molecules present in the MDA, including DARTs and non-DART molecules, can comprise either a covalent or a non-covalent interaction. MDAs can comprise interactions between DART components, including but not limited to protein:protein interactions between Molecular Points (MP:MP or MP-MP), nucleic acid:nucleic acid interactions between Molecular Shafts (MS:MS or MS-MS), protein:nucleic acid interactions between Molecular Points and Molecular Shafts (MP:MS or MP-MS), protein:nucleic acid interactions between Linkage Polypeptides and Molecular Shafts (LP:MS or LP-MS) and other MP:MP, MP:LP, MP:MS, LP:MS, MS:MS, MP-MP, MP-LP, MP-MS, LP-MS, MS-MS interaction combinations. MDAs can also comprise covalent or non-covalent interactions between DART components (e.g., MP, LP, or MS) and a non-DART molecule, including a polypeptide, a small molecule drug, or other molecules. MDAs can optionally comprise more than one of the interactions listed above.

Forming Modular DART Assemblies

Covalent and/or non-covalent intermolecular interactions between two or more DARTs can be mediated by bi- or multi-molecular interactions between individual DART components. The interactions between two non-identical DARTs, for example, D1 and D2 can occur as follows: DART D1 can have MP₁-LP₁-MS₁, and DART b2 can have MP₂-LP₂-MS₂. The interactions between these DARTs can include, but are not limited to, the following: MS₁:MS₂ (or MS₁-MS₂); MP₁:MP₂ (or MP₁-MP₂); MS₁:MP₂ (or MS₁-MP₂); MS₂:MP₁ (or MS₂-MP₁); LP₁:MS₂ (or LP₁-MP₂); LP₁:MP₂ (or LP₁-MP₂) interactions; combinations thereof. Each of these types of intermolecular interactions between DART molecules can be used to form MDAs with useful functions or properties. The following discussion exemplifies the applications of Molecular Point:Molecular Point (MP:MP) or Molecular Shaft:Molecular Shaft (MS:MS) interactions. As will be appreciated, other useful DART interactions can be used in forming MDAs according to the methods described herein or known to the skilled artisan.

MDAs Emerging From MP:MP Interactions

In some embodiments of the present invention, MDAs can be formed via Molecular Point interactions. MDAs whose formation is based on DART MP:MP interactions can include, but are not limited to, the following examples.

In one embodiment, a pair of DARTs can be formed through interactions of their respective Molecular Points. For example, one DART, D1, can have MP₁-LP₁-MS₁. A second DART, D2, can have MP₂-LP₂-MS₂. The interactions between MP₁ and MP2 can form a complex between these two DARTs. The resulting DART complex can be denoted as MS₁-LP₁-MP₁:MP₂-LP₂-MS₂. (The Molecular Shafts MS₁ and MS₂ may or may not interact.) In some embodiments, the interacting Molecular Points can be covalently linked during and/or after MDA assembly to generate a new molecule of the form MS₁-LP₁-MP₁-MP₂-LP₂-MS₂.

In another embodiment, Modular DART assemblies can be formed through interactions between a DART D1 and a Linkage Polypeptide-Molecular Point pair. In this embodiment, DART D1 can be MP₁-LP₁-MS₁, and the Linkage Polypeptide-Molecular Point pair can be LP₂-MP₂ components alone (i.e., without a Molecular Shaft). The molecular complexes that form as a result of the interaction can be of the form LP₂-MP₂:MP₁-LP₁-MS₁ (or LP₂-MP₂-MP₁-LP₁-MS₁). In some cases, more than one Linkage Polypeptide-Molecular Point pair can interact with a given DART (e.g., D1), and/or more than one DART can interact with a Linkage Polypeptide-Molecular Point pair (i.e., LP2-MP2). The interacting Molecular Points can be covalently cross-linked during and/or after this type of MDA complex formation. In addition, the appropriate preMS2 molecule(s) can be added to these complexes to form DARTs from the LP₂-MP₂ pairs. The resulting DART complexes can include those of the form MS₁-LP₁-MP₁:MP₂-LP₂-MS₂ and/or MS₁-LP₁-MP₁-MP₂-LP₂-MS₂.

In another embodiment of the present invention, LP₁-MP₁ pairs can be allowed to contact LP₂-MP₂ pairs to form a mixture harboring LP₁-MP₁ :MP₂-LP₂ (or LP₁-MP₁-MP₂-LP₂) complexes. If so desired, the interacting Molecular Points can be covalently cross-linked during and/or after complex assembly. In addition, the appropriate preMS molecules can be added to these complexes to form DART complexes of the form MS₁-LP₁-MP₁:MP₂-LP₂-MS₂ and/or MS₁-LP₁-MP₁:MP₂-LP₂-MS₂.

The skilled artisan will appreciate that other Molecular Point interactions are possible, including permutations or combinations of those described above. In addition, the methods for forming MDA complexes can be generalized to include interactions between two or more DART and non-DART molecules.

MDAs Emerging From MS:MS Interactions

In another embodiment of the present invention, MDAs can be formed by a variety of Molecular Shaft interactions. MDAs whose formation can be based on DART MS:MS interactions can include, but are not limited to, those described below. The interactions between the Molecular Shafts can be covalent or non-covalent and can be between non-identical (e.g., mutually complementary), or identical Molecular Shafts.

In one embodiment, MDAs can be formed through interactions between a pair of DARTs comprising complementary Molecular Shafts. For example, DART D1 can have MP₁-LP₁-MS₁ and DART D2 can have MP₂-LP₂-MS₂, where MS₁ and MS₂ share sufficient complementarity to one another that they can form a stable heteroduplex. When these two molecules are placed in the appropriate molecular context, a DART complex can be generated of the form MP₁-LP₁-MS₁:MS₂-LP₂-MP₂. In some embodiments of the present invention, the interacting Molecular Shafts can optionally be covalently cross-linked during and/or after MDA complex formation, generating new molecules of the form MP₁-LP₁-MS₁-MS₂-LP₂-MP₂.

In another embodiment of the invention, MDAs can be formed between a DART (e.g., D1=MP₁-LP₁-MS₁) and a preMS (e.g., preMS₂) molecule that can hybridize to a portion of the DART's Molecular Shaft. When these two molecules are allowed to interact, a DART complex of the form preMS₂:MS₁-LP₁-MP₁ can be generated. The interacting preMS₂ molecule and MS₁ can optionally be covalently cross-linked during and/or after their assembly, thus generating a molecule of the form PreMS₂-MS₁-LP₁-MP₁. Also optionally, catalytically active MP₂-LP₂ pairs can be added to this mixture to generate (after an LP₂-mediated covalent linking reaction) DART complexes of the form MP₁-LP₁-MS₁:MS₂-LP₂-MP₂ (or MP₁-LP₁-MS₁-MS₂-LP₂-MP₂).

In another embodiment of the present invention, MDAs can also be formed by contacting preMS molecules, such as, for example, preMS₁ and preMS₂ molecules, that have complementary sequences. Hybridization between preMS₁ and preMS₂ molecules can generate a complex of the form preMS₁:preMS2. These interacting preMS molecules can optionally be covalently cross-linked during and/or after assembly, generating molecules of the form preMS₁-preMS₂. Also optionally, catalytically active MP₁-LP₁ and MP₂-LP₂ pairs can be added to this mixture. These Linkage Polypeptide containing molecules can catalyze the formation of molecules of the form MP₁-LP₁-MS₁:MS₂-LP₂-MP₂ and/or MP₁-LP₁-MS₁-MS₂-LP₂-MP₂.

The skilled artisan will appreciate that other Molecular Shaft interactions are possible, including permutations or combinations of those described above. In addition, the methods for forming MDA complexes can be generalized to include interactions between more than one pair of DARTs and between DARTs and non-DART molecules:

MDA Applications For MP:MP (Or MP-MP) Interactions

MDAs can be used to study the interactions between proteins and the corresponding cellular events mediated by proteins, such as, for example, transcription, signal transduction and enzyme-mediated metabolism. MDAs provide a means for using covalent or non-covalent interactions between components of DARTs to drive the corresponding Molecular Shafts, MS₁ and MS₂, into an intermolecular complex. MDAs can be used to identify, label, substantially purify, retrieve, and/or identify the resulting complex, or components contained therein. MDAs can also be used to identify proteins that bind to a selected protein of interest in vitro and/or in vivo.

In Vitro Binding Partners

MDAs can be used to identify binding partner(s) of a DART in vitro. For example, DART D3 can have MP₃-LP₃-MS₃. Binding partners can be isolated and/or identified by contacting DART D3 with a library of DARTs that have Molecular Points (denoted MPLᵢ) that are candidates for DART D3 MP3 binding partners. The DART library is typically contacted with DART D3 under conditions that limit or inhibit DART shuffling (see supra). During contacting, the MP₃:MPLᵢ interactions can drive the assembly of MDA complexes. If desired, non-covalent interactions between these components can be transformed into covalent interactions by adding chemical cross-linking reagents to the reaction mixture or by applying non-chemical methods such as ultraviolet-mediated cross-linking. The DART D3:library complexes (D3:Li complexes) are then affinity purified (see supra).

The isolated complexes can be analyzed to determine the compositions and properties by methods such as, for example, PCR, mass spectrometry, Western analysis, and other methods known to the skilled artisan. For example, the recovered DART D3 binding partners can be identified based on the nucleic acid sequences within their Molecular Shafts by amplifying MS sequences by PCR using primers complementary to primer annealing sites present in Molecular Shaft library sequences. The amplified products can be isolated and/or sequenced to identify the nucleotide sequences of MP3 binding partners. If sufficient quantities of binding partners are recovered, then direct protein and/or nucleic acid chromatographic and analytical chemical methods (e.g., HPLC-Mass Spectrometry) can used to identify and characterize the binding partners. Alternatively, affinity co-purified complexes can be resolved (before or after amplification) and analyzed on a DARTboard, as described (see supra).

In Vivo Binding Partners

MDAs can also be used to identify binding partner(s) of a DART in vivo. The in vivo binding partners of DART D3 can be identified by co-expressing both DART D3 and a DART library comprising Molecular Points (denoted MPLᵢ) that are candidates for DART D3 MP3 binding partners in host cells. Following this co-expression, interactions between the Molecular Points (e.g., MP₃:MPLᵢ interactions) in vivo can drive the assembly of non-covalent MDA complexes or covalent macromolecular structures. The resulting complexes (e.g., D3:Li or D3-Li) complexes can be affinity purified, such as from whole cell extracts or partially purified extracts. Such affinity purification can be performed as described (supra), for example, using sequences complementary to DART D3 MS3 sequences.

The isolated complexes can be analyzed to determine the compositions and properties by methods such as, for example, PCR, mass spectrometry, Western analysis, and other methods known to the skilled artisan. For example, the recovered DART D3 binding partners can be identified based on the nucleic acid sequences within their Molecular Shafts by amplifying MS sequences by PCR using primers complementary to primer annealing sites present in Molecular Shaft library sequences. The amplified products can be isolated and/or sequenced to identify the nucleotide sequences of MP3 binding partners. If sufficient quantities of binding partners are recovered, then direct protein and/or nucleic acid chromatographic and analytical chemical methods (e.g., HPLC-Mass Spectrometry) can be used to identify and characterize the binding partners. Alternatively, affinity co-purified complexes can be resolved (before or after amplification) and analyzed on a DARTboard, as described (see supra).

Identification of Other Binding Partners

Similar methods can be used to identify other binding partners that interact in vivo or in vitro with a DART Molecular Point. The skilled artisan will appreciate, however, that the principles embodied in these examples can be generalized for other MDA applications. For example, MDA can be used to examine the binding partners for different Molecular Points present in library DARTs at the same time, either in vivo or in vitro. MDA can also be used to identify the library DARTs that do not interact with a DART D3 in vivo or in vitro. MDA can further be used to identify binding partners in library DARTs that interact with a DART D3, where the Molecular Point MP₃ exists in a non-covalent molecular complex with other DART or non-DART molecules. MDA can also be used to identify the DARTs that interact with DART D3 in vivo or in vitro, where the library of Molecular Points, Molecular Shafts and/or Linkage Polypeptides exist in a non-covalent molecular complex with other DARTs or non-DART molecules.

MDA Applications For MS:MS (or MS-MS) Interactions

MDA can also be used to analyze interactions between MDAs that have complementary Molecular Shafts. In particular, when two or more DARTs containing complementary Molecular Shafts are present under conditions that allow Molecular Shaft:Molecular Shaft (MS:MS or MS-MS) duplex assembly, the corresponding Molecular Points can be driven into close physical proximity. This physical proximity between the Molecular Points can be used, for example, for the in vivo or in vitro analysis and/or regulation of intermolecular interactions, and the consequent regulation and manipulation of downstream biochemical and/or biophysical events. The Molecular Points in the complexes can include domains (or regions) of a contiguous polypeptide that are brought into close physical proximity and can reconstitute (or constitute) activity or functions.

MS:MS (or MS-MS) Interactions For Regulating Intermolecular Interactions.

DARTs can be used to drive the formation of intermolecular interactions in vivo or in vitro, thereby creating molecular complexes that possess properties and/or functions not present in the constituent parts. These properties and/or functions can be stimulatory (e.g., stimulating a binding event to a specific substrate), inhibitory (e.g., down regulating an enzyme activity), and the like.

One type of intermolecular interaction is a split-effector interaction. A split-effector interaction occurs when different domains of a molecule, such as a protein, mediate different functions of the molecule. The reconstituted molecule can possess a detectable activity (e.g., biological or chemical) of the original molecule, can possess a new activity, and the like.

Two DARTs, D1 (MP₁-LP-MS₁) and D2 (MP₂-LP-MS₂), can be used. MP1 can possess a function, and MP₂ can possess another function. Typically, neither domain (e.g., MP₁ or MP₂) alone will possess the activity of the intact molecule; MP₁ and MP₂ may not physically associate when MP₁ and MP₂ are separate molecules. These Molecular Points can be brought into proximity by Molecular Shafts, MS₁ and MS₂, of DARTS D1 and D2. Thus, when DARTs D1 and D2 are contacted, the binding of MS₁ and MS₂ can bring MP₁ and MP₂ into close proximity, creating an MDA complex and stimulating the detectable activity.

In one application, two DARTs comprising Molecular Shafts that include mutually complementary nucleic acid sequences are used to modulate signal transduction. For example, a receptor and a ligand are rendered as the Molecular Points of two distinct DARTs, harbor Molecular Shafts that include mutually complementary nucleic acid sequences. When these DARTs are placed in the same in vitro or in vivo context, intermolecular interactions between their complementary Molecular Shafts can drive the receptor and ligand into close proximity, and therefore, stimulate the formation of stable receptor-ligand complex. This complex can propagate a biological signal whose duration or intensity can be distinct from that which would be observed if the relevant Molecular Shaft interactions were not present. Therefore, MDA can be employed to propagate and modulate biological signal transduction events.

In another application, two DARTs comprising Molecular Shafts that include mutually complementary nucleic acid sequences can be used to mediate targeting of a protein medicine to a cell. Briefly, two DARTs harboring Molecular Shaft components that are complementary to one another can be synthesized. The first DART also harbors in its Molecular Point component a protein medicine that lacks the ability to target itself to a specific molecular or cellular context. The second DART harbors in the Molecular Point component a protein that possesses the capacity to recognize and target the DART to a defined molecular context. In this case, that context includes a molecular determinant present on the surface of a target cell. When these two DARTs are placed in the same molecular context, duplex formation between the complementary Molecular Shafts results in the formation of a Modular DART Assembly that possesses the capacity to target the Protein Medicine to the surface of the desired cell as shown.

In another embodiment, two DARTs comprising Molecular Shafts that include mutually complementary nucleic acid sequences can be used regulate the activity of an enzyme. The first DART harbors in the Molecular Point component a protein substrate for a particular enzyme (e.g., protein kinase) activity. The second DART harbors in the Molecular Point component that enzyme activity. When these two DARTs are placed in the same in vitro or in vivo molecular context, Molecular Shaft interactions between the two DARTs facilitate co-localization of their respective Molecular Point components. These Molecular Point interactions can stimulate, for example, the increased phosphorylation of a relevant protein kinase substrate. Therefore, MDA can allow for the generation and regulation of novel molecular complexes with the properties shown.

In yet another application, two DARTs comprising Molecular Shafts that include mutually complementary nucleic acid sequences can be used regulate the activity of an enzyme. The first DART harbors in the Molecular Point component a portion of an enzyme. This portion alone lacks enzymatic activity. The second DART harbors a portion of the same enzyme in the Molecular Point component. This portion, which is non-identical to the portion present in the first DART, also lacks enzymatic activity. However, these portions are selected such that when they are brought into sustained close proximity, the activity of the enzyme is recapitulated. Molecular Shaft interactions between the two DARTs drive relatively stable interactions between their respective Molecular Point components. Specifically, these intermolecular Molecular Point interactions drive the otherwise separated portions of the enzyme into sustained close proximity, stimulating the activity of the enzyme. Therefore, MDA allows for the generation and regulation of molecular complexes with the novel properties shown.

For example, as discussed below for transcriptional activator proteins, DNA binding and transcriptional activation can be mediated by different domains of the protein. The transcriptional activation function of the protein can be reconstituted from the separated domains. Two DARTs, D1 (MP₁-LP-MS₁) and D2 (MP₂-LP-MS₂) can be used. MP₁ can be the DNA binding domain; MP₂ can be the transcriptional activation domain. An MP₁-MP₂ pairing (covalent or non-covalent) can be assembled that possesses the reconstituted activity (e.g., transcriptional activation).

A large variety of MDA complexes for detecting split-effector interactions can be used. One example is the yeast GAL4 system, which is described to illustrate such applications.

MDAs Derived From the Yeast Gal4 Split-Effector Interactions

The yeast protein GAL4 activates the transcription of genes required for metabolism of galactose and melibiose in Saccharomyces cerevisiae. GAL4 binds as a dimer to a consensus palindromic 17-base pair DNA sequence, and is a member of a family of proteins that contain zinc-binding peptide loops that interact specifically with nucleic acid sequences. GAL4 possesses separate functional domains, one binding to DNA and the other activating transcription. These protein domains can be expressed individually from recombinant vectors; GAL4 transcriptional activation can be reconstituted from these separate domains.

MDAs can be used to reconstitute GAL4 activity. GAL4 DNA binding and transcriptional activation domains can be used as Molecular Points on separate DARTs; MP₁ of DART D1 can be the DNA binding domain, and MP₂ of DART D2 can be the transcriptional activation domain. These Molecular Points can be brought into close physical proximity by the binding of complementary Molecular Shafts in DARTs D1 and D2. The close physical proximity of MP₁ and MP₂ can enable the complex to reconstitute GAL4 activity (i.e., the activation of expression of genes located downstream of promoters harboring GAL4 recognition sequences).

Control studies can optionally be performed. For example, control proteins encoding the GAL4 activation domain alone and the GAL4 DNA binding domain alone can be prepared. Similarly, control DARTs, D3 and D4, that are identical to DARTS D1 and D2, respectively, except that their Molecular Shafts (e.g., MSD3 and MSD4) are non-complementary. The control molecules can be used to verify the ability of DARTs D1 and D2, but not the controls (e.g., D3 and D4), to form a non-covalent complex in vitro. This verification process can include co-incubating the appropriate pairs of DARTs together (to drive Molecular Shaft hybridization), resolving complexes formed by gel electrophoresis, and finally detecting the complexes using, for example, antibody probes or primers directed against molecular components of the relevant DARTs. Alternatively, analytical chemical methods (e.g., HPLC-Mass Spectrometry) can be used to resolve and detect the complexes.

DARTs D1 and D2 can be contacted in the presence of a yeast nuclear extracts and/or other requisite factors, with a GAL4 DNA binding site positioned in the promoter region of a reporter gene (e.g., lacZ). During this incubation, the MDA complex containing the D1:D2 complex can activate transcription of the reporter gene by binding to the GAL4 DNA binding site. Typically, transcription will not occur in reaction mixtures harboring D3 and D4 or other control molecules. The signal from the reporter can be detected by a variety of assays including, but not limited to, RT-PCR, northern analysis, RNA protection analysis, by direct visualization by gel electrophoresis and nucleic acid staining, and/or by activity assays (e.g., β-galactosidase assays).

The Utility of GAL4 MDA Systems.

The GAL4 MDA system described above can be used to generate MDA tools for a wide variety of purposes. Such tools can be used, for example, to identify novel nucleic acid binding proteins, to identify transcriptional activation domains in vitro and in vivo, and the like. In one embodiment, a library of DARTs, D_{z} (MP_{z}-LP_{z}-MS_{z}), can be screened to uncover novel transcriptional activation domains, and for revealing novel DNA binding proteins.

Novel transcriptional activation domains can be identified by, for example, using the GAL4 DNA binding domain as the Molecular Point of DART, DART A1. The interactions of DART A1 with the Molecular Points of the library Dz can be screened for those that activate transcription of a reporter gene construct (e.g., having a GAL4 DNA binding site positioned in the promoter region of a reporter gene, such as lacZ). The assay can be performed in vitro or in vivo. The resulting complexes can include the assembly of Dz:A1 MDA complexes. Such complexes can include MP_{z}-LP_{z}-MS_{z}:MS_{A1}-LP_{A1}-MP_{A1} and/or MPz-LPz-MSz-MS_{A1}-LP_{A1}-MP_{A1}. By this method, it can be possible to isolate and/or identify DARTs of library Z that have the capacity to act as GAL4 transactivators, that is, to stimulate the transcription of a reporter gene located downstream of GAL4 DNA binding sequences.

Conversely, novel DNA binding proteins can be identified using a DART library, D_{z} (MP_{z}-LP_{z}-MS_{z}), and the GAL4 transcriptional activation domain in a DART, DART A2. The interactions between the library DARTs and DART A2 can be screened in vivo and/or in vitro for those that stimulate transcription of a reporter gene. In particular, the interactions allows the assembly of D_{z}:A2 MDA complexes. The resulting complexes can include MP_{z}-LP_{z}-MS_{z}:MS_{A2}-LP_{A2}-MP_{A2} and/or MP_{z}-LP_{z}-MS_{z}-MS_{A2}-LP_{A2}-MP_{A2}. This assay provides for the identification of complexes that stimulate the transcription of downstream reporter genes; thus, it can be possible to isolate and/or identify elements of library Dz that can act as DNA binding proteins, that is to bind regulatory DNA sequences located in promoter regions upstream of the reporter genes to be transactivated; and to stimulate the transcription of these downstream reporter genes.

Generality of MDA

Although the above examples relate GAL4-derived MDAs, similar MDAs can be used in vivo (e.g., in Saccharomyces cerevisiae or other host cells) to regulate the transduction of a wide variety of biochemical signals. Other GAL4- and/or non-GAL4-MDA systems with similar properties can be devised for use in in vitro, or in vivo (e.g., prokaryotic and/or eukaryotic systems. These systems can include elements derived from a wide variety of naturally-occurring or specifically engineered intermolecular interactions, including but not limited to the following: protein ligand:receptor interactions interactions; drug:antibody interactions; protein substrate:enzyme interactions (e.g., protein kinase substrate:protein kinase interactions); enzyme:protein inhibitor interactions; the analysis of two-domain enzymes whose structure/activity accords with split-effector properties described above; the analysis of two-domain regulatory proteins whose structure/activity accords with split-effector properties described above; and the like.

Regulating Assembly and Disassembly of MDAs

MDA complexes can be subjected to a variety of regulatory mechanisms, both in vitro and in vivo, and these regulatory mechanisms can be exploited for a variety of purposes. In particular, the activities of MDA complexes can be regulated by manipulating their assembly and disassembly.

Assembly

MDA complex assembly can be regulated in vivo by a variety of methods, including manipulating the synthesis of the constituent DARTs (and/or their components). Similarly, MDAs can be regulated in vitro by manipulating the time at which the constituent DARTs (and/or their components) are contacted. Regulating the assembly of the complexes can also be useful for creating temporal windows for analysis. For example, MDAs can be used as an analytical or regulatory tool at a particular time in vivo (e.g., at a particular stage in the cell cycle or after the addition of a drug). DARTs can be formed and/or delivered in (or to) the cells at the time of interest. Inducible promoters can be used, for example, to drive the expression constructs encoding the DARTs (e.g., LP-MP expression constructs). Similar constructs can be used for the temporal regulation of MDA synthesis and function in vitro.

Disassembly

MDA complexes can be regulated in vivo and/or in vitro by manipulating either the disassembly of the constituent DARTs (or their components) and/or the disassembly all or part of the MDA complexes. For example, MDA disassembly can be regulated by hydrolysis and/or destruction of entire DARTs (or DART components) and/or non-DART molecules mediating the interaction, or by subjecting the complex to conditions that reversibly or irreversibly disrupt the interactions maintaining the integrity of the assembly. Methods for disassembling interacting DARTs can be generalized to more complex MDAs and/or non-DART molecular constituents.

MDAs in which Molecular Point interactions (e.g., MP:MP or MP-MP) mediate the assembly can be disassembled in vitro and/or in vivo. In vitro mechanisms for disassembling MP:MP interactions include, but are not limited to, raising the temperature, adding a peptide or other molecule that disrupts the interaction, adding a protease to hydrolyze Molecular Points mediating the MDA interaction, and other methods known to the skilled artisan. A combination of these methods can also be used. These methods can be adapted to disrupt MDAs in vivo. For example, Molecular Point interactions (MP:MP or MP-MP) can be disrupted in vivo by a variety of means, including, but not limited to adding a peptide or other molecule that disrupts the interaction, co-expressing in (or delivering to) cells harboring the MDA complexes a polypeptide that promotes the disassembly of the MDA, co-expressing in (or delivering to) cells harboring MDA complexes a protease known to hydrolyze Molecular Points mediating the interaction, and the like. A combination of these methods can also be used. Some of the above methods for disassembling MDA complexes can be reversible. For example, if adding a small molecule disrupts the in vitro interaction, then removing it, for example by dialysis or gel filtration chromatography, can restore the MDA complex.

Regulating the assembly and/or disassembly of the complex or structure can be useful for creating temporal or spatial windows of analysis and/or regulation. For example, if the presence of one or more DART and/or non-DART molecules in a MDA complex prevent MDA targeting to a desired location, then disassociating those inhibitory molecules from the MDA complex can allow the remaining constituents to target to the desired location.

Disassembling MS:MS Interacting MDAs

MDAs comprising MS:MS interactions between DARTs can be disassembled in vitro and/or in vivo. The interaction between complementary Molecular Shafts, MS1 and MS2, can be disrupted by mechanisms that disrupt the heteroduplex. These mechanisms include, but are not limited to the addition of a restriction enzyme that recognizes one or more restriction sites present in the duplex DNA, raising the temperature of the reaction mixture above the melting temperature (Tm) of the duplex DNA, adding an exonuclease that hydrolyzes all of part of the complementary Molecular Shafts, and the like. Some of these steps can be reversible. For example, if a temperature change is used to disrupt the interaction, then lowering the temperature (e.g., below the Tₘ) can promote MS:MS duplex formation and MDA complex formation. Similarly, adding a ligase to mixtures harboring MDA complexes or structures that have been disassembled by restriction endonuclease cleavage can reform the MDA.

In some aspects, it can be desirable to inhibit the reversibility of the reaction thereby preventing MDA complexes from forming. For example, MS:MS duplex formation can be prevented by adding a large excess of free nucleic acids harboring sequences complementary to one or both of the Molecular Shafts. Similarly, ligation can be prevented by modifying the ends of target nucleic acids.

DART Gene Therapy

In another aspect, one or more nucleic acids encoding a DART or a component of a DART are administered as a form of gene therapy. Gene therapy refers to therapy performed by the administration to a subject of an expressed or expressible nucleic acid. In this embodiment of the invention, the nucleic acids produce a DART that mediates a therapeutic effect. Any of the methods for gene therapy available in the art can be used according to the present invention. Exemplary methods are described below.

For general reviews of the methods of gene therapy, see, Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; Morgan and Anderson, 1993, Ann. Rev. Biochem. 62:191-217; May, 1993, TIBTECH 1, 1(5):155-215. Methods commonly known in the art of recombinant DNA technology which can be used are described in Ausubel et al. (supra); and Kriegler, Gene Transfer and Expression, A Laboratory Manual, Stockton Press, NY (1990).

In a typical embodiment, the therapeutic comprises nucleic acid sequences encoding an RNase DART, the nucleic acid sequences being part of expression vectors that express the Linkage Polypeptide and preMS components. Such nucleic acid sequences have promoters operably linked to the Linkage Polypeptide- and preMS-coding region, the promoter being inducible or constitutive, and, optionally, tissue-specific (for examples of inducible, constitutive and tissue-specific promoters, see supra). In another embodiment, nucleic acid molecules can be used in which the DART coding sequences and any other desired sequences are flanked by regions that promote homologous recombination at a desired site in the genome, thus providing for intrachromosomal expression of the DART-encoding nucleic acids (see, e.g., Koller and Smithies, 1989, Proc. Natl. Acad. Sci. USA 86:8932-8935; Zijlstra et al., 1989, Nature 342:435-438).

In an exemplary embodiment, a nucleic acid encoding an RNase DART that can be delivered to a patient via a gene therapy vehicle can comprise, in a 5′ to 3′ direction, (i) a tissue specific promoter; (ii) a nucleic acid sequence encoding a Molecular Point that is an RNase, e.g. RNase H, that catalyses the degradation of double-stranded (but not single-stranded) RNA molecules, the Molecular Point coding sequence being in frame with (iii) a nucleic acid sequence encoding a Linkage Polypeptide; (iv) a Recognition Sequence Motif; (v) a nucleic acid sequence corresponding to the gene whose RNA is to be down-regulated (vi) and, optionally, a ribosomal pause sequence. Following transcription of this sequence, translation of the RNase H-LP pair, and formation of a covalent linkage between the LP and the RNA produced by the gene therapy vector (which is this instance is also a preMS), a DART is formed in which the Molecular Point comprises an RNase molecule and the Molecular Shaft comprises a nucleic acid that is complementary to the Molecular Target RNA whose destruction or reduced abundance is desired.

In another embodiment, a nucleic acid encoding an RNase DART that can be delivered to a patient via a gene therapy vehicle can comprise, in a 5′ to 3′ direction, (i) a tissue specific promoter; (ii) a nucleic acid sequence encoding a Linkage Polypeptide, the Linkage Polypeptide coding sequence being in frame with (iii) a nucleic acid sequence encoding a Molecular Point that is an RNase, e.g. RNase H, that catalyses the degradation of double-stranded (but not single-stranded) RNA molecules; (iv) a Recognition Sequence Motif; (v) a nucleic acid sequence corresponding to the gene whose RNA is to be down-regulated (vi) and, optionally, ribosomal pause sequence. Following transcription of this sequence, translation of the RNase H-LP pair, and formation of a covalent linkage between the LP and the RNA produced by the gene therapy vector (which is this instance is also a preMS), a DART is formed in which the Molecular Point comprises an RNase molecule and the Molecular Shaft comprises a nucleic acid that is complementary to the Molecular Target RNA whose destruction or reduced abundance is desired.

In another exemplary embodiment, two nucleic acids can be delivered simultaneously to the same cell. One nucleic acid can comprise a sequence encoding the MP-LP, or RNase-LP, pair under the control of a suitable promoter, and a second nucleic acid encoding the preMS under the control of a suitable promoter. The preMS pair comprises, in a 5′ to 3′ orientation, a Recognition Sequence Motif and a Molecular Shaft complementary to the Molecular Target RNA of choice. Following synthesis of the RNase-LP pair and production of the preMS molecule, a covalent linkage reaction between the RNase-LP and preMS results in a DART comprising an RNase, an LP, and a MS that is complementary to the Molecular Target RNA, and can lead to the destruction of all or part of the Molecular Target RNA population in a cell or in another setting.

RNase DARTS can optionally comprise NLS that target them to the nucleus, in which case the target RNA can be destroyed in the nucleus rather than the cytoplasm.

Delivery of the nucleic acids into a patient can be either direct, in which case a patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences can be directly administered in vivo, where the DART coding sequences can be expressed to produce the encoded product (see supra). This can be accomplished by any of numerous methods known in the art, for example by constructing them as part of an appropriate nucleic acid expression vector and administering the vector so that the nucleic acid sequences become intracellular. Gene therapy vectors can be administered by infection using defective or attenuated retrovirals or other viral vectors (see, e.g., U.S. Pat. No. 4,980,286); direct injection of naked DNA; use of microparticle bombardment (e.g., a gene gun; Biolistic, Dupont); coating with lipids or cell-surface receptors or transfecting agents; encapsulation in liposomes, microparticles, or microcapsules; administration following attachment to a peptide which is known to enter the nucleus; administration in linkage to a ligand subject to receptor-mediated endocytosis (see, e.g., Wu and Wu, 1987, J. Biol. Chem. 262:44294432) (which can be used to target cell types specifically expressing the receptors); and the like. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO 92/20316; WO 93/14188, and WO 93/20221). Alternatively, the nucleic acid can be introduced intracellularly and incorporated within host cell DNA for expression by homologous recombination (see, e.g., Koller and Smithies, 1989, Proc. Natl. Acad. Sci. USA 86:8932-8935; Zijlstra et al., 1989, Nature 342:435-438).

In a specific embodiment, viral vectors that contain nucleic acid sequences encoding a DART can be used. For example, a retroviral vector can be used (see, e.g., Miller et al., 1993, Meth. Enzymol. 217:581-599). These retroviral vectors contain the components necessary for the correct packaging of the viral genome and integration into the host cell DNA. The DART-encoding nucleic acid sequences to be used in gene therapy can be cloned into one or more vectors, thereby facilitating delivery of the gene into a patient. More detail about retroviral vectors can be found in Boesen et al., 1994, Biotherapy 6:29 1-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Another approach to gene therapy involves transferring a nucleic acid, e.g., a DART-encoding nucleic acid, to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred nucleic acid. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell mediated gene transfer, spheroplast fusion, and the like. Numerous techniques are known in the art for the introduction of foreign nucleic acids into cells (see, e.g., Loeffler and Behr, 1993, Meth. Enzymol. 217:599-618; Cohen et al., 1993, Meth. Enzymol. 217:618-644; Cline, 1985, Pharmac. Ther. 29:69-92), and can be used in accordance with the present invention, provided that the necessary developmental and physiological functions of the recipient cells are not disrupted.

The resulting recombinant cells can be delivered to a patient by various methods known in the art. Recombinant blood cells (e.g., hematopoietic stem or progenitor cells) are typically administered intravenously. The amount of cells envisioned for use depends on the desired effect, patient state, etc., and can be determined by one skilled in the art.

In an embodiment in which recombinant cells are used in gene therapy, DART-encoding nucleic acid sequences can be introduced into the cells such that they are expressible by the cells or their progeny, and the recombinant cells can be administered in vivo for therapeutic effect. In a specific embodiment, stem or progenitor cells can be used. Any stem and/or progenitor cells which can be isolated and maintained in vitro can potentially be used in accordance with this embodiment of the present invention (see, e.g. PCT Publication WO 94/08598; Stemple and Anderson, 1992, Cell 71:973-985; Rheinwald, 1980, Meth. Cell Bio. 21 A:229; and Pittelkow and Scott, 1986, Mayo Clinic Proc. 61:771).

Kits

The present invention further provides kits comprising in one or more containers the novel compositions and/or reagents for practicing the methods disclosed herein. Optionally associated with such container(s) can be a notice in the form prescribed by a governmental agency regulating the manufacture, use or sale of pharmaceuticals or biological products, which notice reflects approval by the agency of manufacture, use or sale for human administration.

In one embodiment, the invention provides a kit comprising in one or more containers one or more homogenous populations of DARTs (i.e., a population of a single DART species). In another embodiment, the population of DARTs is purified. The population of DARTs can comprise RNA, DNA or RNA-DNA Molecular Shafts.

In another embodiment, the invention provides a kit comprising in one or more containers DART-, MP-LP- and/or preMS-encoding nucleic acids. In one mode of the embodiment, a kit of the invention comprises one or more nucleic acids, each encoding a self-referential DART. In yet another mode of the embodiment, a kit of the invention comprises one or more nucleic acids which encodes a preMS and one or more nucleic acids which encodes a MP-LP pair. The nucleic acid can be suitable for in vivo or in vitro DART synthesis and/or assembly. For example, the nucleic acid encoding a DART or MP-LP can be operatively linked to a promoter suitable for in vivo transcription or for in vitro transcription. Optionally included in such kits can be reagents that facilitate DART formation reaction in vitro (for example, a magnesium-containing buffer and/or a VirD1 expression construct when the encoded LP is VirD2) or in vivo (for example a cell suitable for expression of the DART or DART component).

The MP-LP in a kit of the invention or encoded by the nucleic acid in a kit of the invention can further include, for example, (in the MP portion, the LP portion, or both) an epitope tag (e.g., for detection/affinity selection) or a protease cleavage site. A kit comprising such an MP-LP pair (or coding region) can further comprise reagents for detection or affinity purification of the epitope tag, or a protease, respectively.

In yet other aspect of the invention, kits comprising in one or more containers a DART library comprising a heterogeneous population of DARTs (i.e., a population of DARTs containing multiple DART species) or a heterogeneous population of DART-encoding nucleic acids (i.e., a population nucleic acids encoding multiple DART species) are provided. In one embodiment, the MPs of the heterogenous DART population can be variants or mutants of a single protein (e.g., for directed molecular evolution applications).

In yet other aspects of the invention, kits are provided which comprise in one or more containers a nucleic acid vector encoding an LP, in which adjacent to the LP coding sequence is one or more restriction enzyme recognition sites into which a library of nucleic acids encoding MPs can be inserted in frame with the LP coding sequence.

The present invention further provides a kit comprising in one or more containers a population of cells capable of expressing DARTs. The cells can express one or more species of DARTs and, in a typical embodiment, express a DART library. In a typical mode of the embodiment, most of the cells express only one DART species. The cells can express the DARTs constitutively or under the control of an inducible promoter. The cells can comprise one or more vectors encoding a DART or DART component, and/or can comprise chromosomally-integrated sequences encoding a DART or DART component.

The present invention provides a kit comprising in one or more containers a DART probe, the DART probe comprising a detectably-labeled MS and/or a detectably-labeled MP. In a typical embodiment, the detectable label is fluorescent.

The present invention further provides a kit comprising in one or more containers one or more nucleic acids encoding a DART probe, the DART probe comprising a detectably-labeled MS and/or a detectably-labeled MP. In one embodiment, the nucleic acid can be an expression vector. Such a kit can optionally further comprise detectably labeled nucleotides or reagents for the purification of the DART probe.

In yet other embodiments of the invention, kits comprising in one or more containers a DARTboard or reagents for the production of DARTboards are provided. In one embodiment, a kit of the invention comprises a pre-fabricated DARTboard, the prefabricated DARTboard comprising DARTs on a DNA microarray. In another embodiment, a kit of the invention comprises template DNA pre-fabricated microarrays.

In yet other embodiments of the invention, kits comprising in one or more containers a diagnostic DART or reagents for the production of a diagnostic DART (e.g., one or more nucleic acids encoding a diagnostic DART or a component thereof). In one embodiment, the kit comprises diagnostic DARTs in a pre-fabricated DARTboard.

Pharmaceutical DART Compositions

The present invention further provides pharmaceutical compositions comprising DARTs or DART-encoding nucleic acids for administration to a subject. The DART- or DART-encoding nucleic acid is typically at least 70%, more typically 80%, yet more typically 90%, yet more typically 95% and yet most typically at least 99% free from substances that limit its effect or produce undesired side-effects. The subject is typically an animal, including but not limited to animals such as cows, pigs, horses, chickens, cats, dogs, etc., and is typically a mammal, and most typically human.

Various delivery systems are known and can be used to administer DARTs or DART-encoding nucleic acids, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction include but are not limited to intradermal, intramuscular, intraperitoneal, intravenous, subcutaneous, intranasal, epidural, and oral routes. DARTs or DART-encoding nucleic acids can be administered by any convenient route, for example by infusion or bolus injection, by absorption through epithelial or mucocutaneous linings (e.g., oral mucosa, rectal and intestinal mucosa, etc.) and can be administered together with other biologically active agents. Administration can be systemic or local.

In a specific embodiment, it may be desirable to administer DARTs or DART-encoding nucleic acids by injection, by means of a catheter, by means of a suppository, or by means of an implant, the implant being of a porous, non-porous, or gelatinous material, including a membrane, such as a sialastic membrane, or a fiber. Typically, when administering a DART, care can be taken to use materials to which the DART does not absorb.

In another embodiment, the compound or composition can be delivered in a vesicle, in particular a liposome (see, e.g., Langer, 1990, Science 249:1527-1533; Treat et al., 1989, in Liposomes in the Therapy of infectious Disease and Cancer, Lopez-Berestein and Fidler (eds.), Liss, New York, pp 353-365; Lopez-Berestein, ibid., pp. 317-327; see generally, ibid.).

In yet another embodiment, the compound or composition can be delivered in a controlled release system. In one embodiment, a pump may be used (see, e.g., Langer, Sefton, 1989, CRC Crit. Ref. Biomed. Eng. 14:201; Buchwald et al., 1980, Surgery 88:507; Saudek et al., 1989, N. Engl. J Med. 321:574). In another embodiment, polymeric materials can be used (see, e.g., Medical Applications of Controlled Release, 1974, Langer and Wise (eds.), CRC Pres., Boca Raton, Fla.; Controlled Drug Bioavailability, Drug Product Design and Performance, 1984, Smolen and Ball (eds.), Wiley, New York; Ranger and Peppas, 1983, Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

Other controlled release systems are discussed in the review by Langer, 1990, Science 249:1527-1533.

In a specific embodiment where one or more DART-encoding nucleic acids are administered, the nucleic acid(s) can be administered via a gene therapy vector, as described supra.

As mentioned above, pharmaceutical compositions comprising a therapeutically effective amount of DART or DART-encoding nucleic acid(s), and a pharmaceutically acceptable carrier, are provided. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a typical carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions can contain a therapeutically effective amount of a DART or DART-encoding nucleic acid(s), typically in purified form, together with a suitable amount of carrier so as to provide a form for proper administration to the patient. The formulation should suit the mode of administration.

In a typical embodiment, the pharmaceutical of the invention is formulated in accordance with routine procedures as a pharmaceutical composition adapted for intravenous administration to human beings. Typically, compositions for intravenous administration can be solutions in sterile isotonic aqueous buffer. Where necessary, the pharmaceutical of the invention can also include a solubilizing agent and a local anesthetic such as lignocaine to ease pain at the site of the injection. Generally, the ingredients can be supplied either separately or mixed together in unit dosage form, for example, as a dry lyophilized powder or water free concentrate in a hermetically sealed container such as an ampoule or sachette indicating the quantity of active agent. Where the pharmaceutical of the invention is to be administered by infusion, it can be dispensed with an infusion bottle containing sterile pharmaceutical grade water or saline. Where the pharmaceutical of the invention is administered by injection, an ampoule of sterile water for injection or saline can be provided so that the ingredients may be mixed prior to administration.

This invention is further illustrated by the following examples which should not be construed as limiting. The contents of all references, patents and published patent applications cited throughout this application are hereby incorporated by reference in their entireties.

### EXAMPLE

### DART Synthesis In Vitro

DART Synthesis In Vitro

DARTs were synthesized and examined to verify their composition. A catalytically active MP-LP pair, His6D2, was synthesized and coupled to various preMS Molecule (e.g., oligonucleotides). The covalent linkage reaction of His6D2 was examined in simple solutions and in the presence of a complex mixture of biological molecules that modeled in vivo DART synthesis conditions. Quantitative studies confirmed the stoichiometry of the covalent linkage reaction: preMS+MP-LP=MP-LP-MS (DART)+postMS.

Coincident detection methods confirmed the MP-LP-MS composition of the DARTs and demonstrated that all three of these components were available to bind to other molecules. Specifically, DARTs could be detected using anti-His6 and anti-His6D2 antibodies as well as streptavidin—Horse Radish Peroxidase conjugate (SA-HRP). In addition, a large fraction of the His6D2 molecules that were purified were competent to form DARTs. Moreover, DART complexes were detected and different DART species exhibited different MP, LP, and MS binding availability profiles.

These studies also confirmed that an MP-LP pair was capable of catalyzing a covalent linkage reaction between a Molecular Shaft (MS₁) and a postMS₂ molecule. This covalent linkage reaction can be used for DART shuffling and DARTex. These studies also demonstrated that the Molecular Shaft of a DART could hybridize to complementary nucleic acid molecules, but did not hybridize to non-complementary nucleic acid molecules.

Materials And Methods

Plasmids

Plasmid pET-D2, encoding VirD2 with an amino-terminal His6 tag within the Stratagene pET28 vector, was used as a source of the linkage polypeptide His6D2. The pET28-D2 vector was sequenced to confirm the sequence of the amino-terminal tag and that it did not contain any mutations. The amino acids prior to the VirD2 starting methionine were: MGSSHHHHHHSSGLVPRGSHMASMTGGQQMGRGSEF (SEQ ID NO: 33). This sequence constitutes the Molecular Point (MP). A similar vector was used to express a VirE2—His6 polypeptide, denoted His6E2.

Oligonucleotides

Oligonucleotides ordered from Gibco BRL were resuspended in water and treated according to standard protocols. Some of these oligonucleotides served as preMS molecules in different studies. Other oligonucleotides were designed to hybridize to all or part of a Molecular Shaft and/or a preMS Molecule (see Table 3). As required, oligonucleotides were labeled with gamma-³²P-ATP at their 5′ termini using T4 polynucleotide kinase and standard protocols. As required, oligonucleotides were labeled with alpha-³²P-dCTP, or biotinylated dideoxy UTP (biotin-ddUTP) at their 3′ termini using terminal transferase and standard protocols.

Protein Purification

Plasmid pET28-D2 (Deng et al., 1998, Proc. Natl. Acad. Sci. USA. 95:7040-45) in E. coli strain BL21 was grown on medium containing kanamycin and subjected to the non-denaturing protein purification protocol outlined in the QiaExpress kit (Qiagen). Fractions of this purification were analyzed. Elution fraction #3 contained His6D2. A Slide-a-lyzer was used to dialyze the His6D2 sample into D2 buffer, which contained of 20 mM Tris HCl, 50 mM NaCl, 1 mM EDTA, 15% glycerol, pH 8.8. Purified, dialyzed His6D2 protein was frozen in small aliquots in liquid nitrogen and stored at −80° C.

HIS6D2 Covalent Linkage Reaction Activity Assays

For the covalent linkage reaction, buffer D2 was supplemented with 5 mM MgCl₂. Reactions were incubated for sixty minutes at 37° C. Reactions were stopped with 50 mM EDTA and/or the addition of loading dye containing SDS.

Analysis of Proteins

Fractions from the His6D2 purification scheme described above were subjected to acrylamide gel electrophoresis using BioRAD acrylamide gels run with Laemmli buffer and loading dyes used in standard protocols. The concentration of His6D2 was measured using BioRAD protein quantification reagents. Each 1 μl of dialyzed His6D2 stock contained 4.5 picomoles of His6D2, and most covalent linkage reactions contained 2.7 picomoles (0.6 μl) of His6D2.

Detection of Radionuclides

Radionuclides bound to or part of DARTs or other molecules were detected following exposure of a sample to a phosphorimager screen, which was then scanned using Molecular Dynamics hardware and analyzed with appropriate software.

Western Analysis

Standard methods were employed for the electrochemical transfer of proteins from gels to membranes. A monoclonal anti-His6 antibody was used in Western analysis to detect the His6 epitope in His6D2. A rabbit polyclonal antibody raised against His6D2 was used to detect the His6D2 protein (Deng et al., 1998, Proc. Natl. Acad. Sci. USA. 95:7040-45). Streptavidin-conjugated horse radish peroxidase (SA-HRP) was used in Western blots to detect molecules and/or complexes of molecules containing biotin.

Results

DART Components—PreMS Molecules

Oligonucleotides containing a VirD2 Recognition Sequence Motif (RS) were used as preMS molecules for DART synthesis. Other oligonucleotides served a variety of purposes (see Table 3).

DART Components—Purification of HIS6D2 (MP-LP)

To synthesize DARTs, an MP-LP polypeptide was purified. The expression vector pET28-D2 encodes the Agrobacterium tumefaciens VirD2 Linkage Polypeptide (LP) and a thirty-six amino acid tag that included a stretch of six histidines useful for protein purification and antibody binding (a Molecular Point). This protein was purified under non-denaturing conditions to preserve the activity of the LP component. This MP-LP pair is denoted His6D2.

The His6 epitope tag was exploited to purify His6D2 under non-denaturing conditions using Ni—NTA agarose. Coomassie staining revealed that purified fractions contained a single primary band of the expected size. Western analysis with anti-His6 and anti-D2 antibodies confirmed the identity of this band as His6D2. The His6D2 protein was dialyzed into D2 buffer. His6D2 retained its activity even after being frozen at −80° C. and thawed. Small aliquots of purified His6D2 were frozen and thawed, as required to perform each study.

Covalent Linkage Reaction Activity of HIS6D2 (DART Synthesis)

The covalent linkage reaction resulting in preMS cleavage and DART synthesis was as follows: preMS+MP-LP=MP-LP-MS (DART)+postMS. The progression of this reaction was assessed experimentally by several means.

In one assay, a Recognition Sequence Motif (RS)-containing oligonucleotide labeled at the 5′ terminus with ³²P-ATP served as a preMS Molecule. Because the postMS Molecules and DARTs were expected to be produced in equimolar amounts, the production of postMS Molecules was used to monitor DART production. In particular, the reduced abundance of the preMS molecules and the corresponding increase in the abundance of postMS molecules was observed.

In a second assay, the DART product was detected directly by Western analysis with antibodies directed against His6 and/or His6D2. Detection was also performed using a label, such as biotin or a radionuclide, linked to the Molecular Shaft or an oligonucleotide hybridized to the Molecular Shaft. All of these methods yielded results that were consistent with each another.

Covalent Linkage Activity In Purification Fractions

Different fractions of the His6D2 purification were tested for covalent linkage activity. The elution fractions containing His6D2 exhibited activity whereas other fractions did not. Elution fractions from the purification of His6E2, a fusion protein containing the His6 motif fused to VirE2, did not did not exhibit this covalent linkage activity. These data indicated that covalent linkage reaction activity could be attributed to His6D2 and not to another protein from host cells BL2 1 that bound the Ni-NTA purification matrix. An aliquot of His6D2 that was frozen in liquid nitrogen and thawed exhibited covalent linkage activity as well.

DART Synthesis In A Complex Solution Containing Proteins And Other Biomolecules

To confirm that DARTs could be synthesized in a complex solution containing proteins and other biomolecules, His6D2 covalent linkage activity was assayed under a variety of reaction conditions. Covalent linkage reaction activity was not detected when His6D2 and preMS molecules were co-incubated in the first wash fraction from the His6E2 purification, which should contain a myriad of bacterial-derived proteins and molecules that include His6E2 (His6-tagged VirE2), but not His6D2. The preMS molecules were partially degraded under these conditions. In a negative control, no His6D2 activity was observed in a control reaction containing EDTA. In contrast, His6D2 exhibited covalent linkage reaction activity when incubated with preMS molecules in the wash buffer alone. Thus, one or more molecules present in the His6E2 wash fraction degraded the preMS molecules and possibly inhibited the covalent linkage reaction. When this fraction was boiled prior to the addition of the preMS molecules and His6D2, cleavage activity was detected. Oligonucleotide 53 cleavage was an indicator of DART synthesis. These data demonstrated that DARTs were synthesized in a complex solution containing many proteins and other biological molecules derived from a living cell.

### EXAMPLE

### DART Synthesis In Live Cells

DART synthesis was performed inside E. coli cells. Specifically, plasmids comprising VirD2 recognition sequences and sequences encoding VirD2 and VirD1 were employed to synthesize DARTs inside E. coli cells. The resulting DARTs were affinity purified and detected by PCR amplification of their linked Molecular Shafts.

Plasmid pCB301, comprising VirD2 recognition sequences, served as the preMS (Xiang et al., 1999, Plant Mol Biol. 40:711-717). Plasmids encoding VirD2 and/or VirD1 under control of the arabinose-inducible AraC promoter provided the MP-LP and the linking protein VirD1. Various combinations of these plasmids were introduced into TOP10 E. coli cells (see Invitrogen pBAD TOPO TA Expression Kit manual version J 2001 and references therein).

The resulting bacterial cells were induced two hours with 0.4% arabinose and lysed without sonication in 150 mM NaCl, 10 mM EDTA, 25 mM Tris-HCl pH 8.0, and 1% triton X-100. Samples were vortexed, clarified by centrifugation, and subjected to Nickel-NTA agarose affinity purification as described (see Qiagen QlA expressionist handbook Third edition 1997 and references therein) with the following modifications: 10% glycerol and 1% triton X-100 were added to wash 2. Sodium chloride was added to wash 3 to a final concentration of 1.5 M. Finally, DARTs present in eluates were detected by PCR amplification of their Molecular Shafts using primers 601 and 602, which are complementary to the Molecular Shaft derived from the pCB301 preMS. (Table 2.) The following samples were analyzed for DART synthesis: 1. Plasmid pCB301 (preMS alone, no MP-LP); 2. Plasmid pCB301 and plasmid B6L1 encoding VirD1 and VirD2:His6 (MP-LP, linking cofactor VirD1, and preMS); 3. Plasmid B6L1 encoding VirD1 and VirD2:His6 (MP-LP alone, no preMS); 4. Plasmid CB2L2 encoding VirD2:His6, pCB301 (MP-LP, preMS, missing linking cofactor VirD1); and 5. Plasmid B6L1 encoding VirD1 and VirD2:His6 (MP-LP, linking cofactor VirD1, no preMS).

A PCR product of the expected size was obtained in sample 2 but not in any of the four negative control samples. Thus, E. coli cells containing preMS, VirD1, and VirD2 produced DARTs whereas samples lacking one or more of these components did not. These data indicate that DARTs were synthesized inside E. coli cells.

### EXAMPLE

### Varying DART Synthesis Conditions

The present example shows how the parameters of DART synthesis reactions can be varied to allow the optimization of the DART synthesis process. While exemplified for DARTs containing VirD2 as a Linkage Polypeptide, these manipulations can be carried out to optimize DART formation for any Linkage Polypeptide.

His6D2 Dilution Series Activity Profile

The covalent linkage reaction resulting in preMS molecule cleavage and DART synthesis was as follows: preMS+MP-LP=MP-LP-MS (DART)+postMS. The equilibrium concentrations of covalent linkage reaction products and reactants can be predicted from the initial reactant concentrations. Specifically, given starting conditions where [preMS]>[MP-LP], at equilibrium [MP-LP]=[DART]=[postMS]. If instead [preMS]<[MP-LP], then at equilibrium [preMS]=[DART]=[postMS]. If [preMS]=[MP-LP], then at equilibrium [MP-LP]=[DART]=[preMS]=[postMS]. Based on the reaction formula and the predicted equilibrium conditions, when one component was in excess (e.g., His6D2) then at equilibrium half of the starting amount of the other component (e.g., preMS molecule) would remain and the other half would be converted into the DARTs and postMS molecule.

To test whether the stoichiometry of the covalent linkage reaction matched the predictions, a dilution series of His6D2 protein was prepared and assayed for covalent linkage reaction activity. Ten serial two-fold dilutions were prepared. The amount of postMS reaction product was reduced in the ⅛ dilution of His6D2. This occurred when the concentration of His6D2 dropped below 0.34 picomoles when the probe concentration was 0.25 picomoles. This was consistent with the predicted covalent linkage reaction stoichiometry, whereby one MP-LP pair reacts with one preMS molecule to generate one DART (MP-LP-MS) and one postMS molecule. Moreover, it indicated that a large fraction of the purified His6D2 protein exhibited covalent linkage reaction activity.

The State of PreMS Molecules Affects the Covalent Linkage Reaction

The following study tested the ability of His6D2 to perform the covalent linkage reaction on single-stranded and double DNA templates (preMS Molecules) containing the T-DNA right border (RS).

Different combinations of oligonucleotides were mixed in the absence of His6D2 and incubated under conditions that permitted complementary oligonucleotides to pair with one another to form DNA duplexes. 0.25 picomoles of 5′ kinased oligonucleotide 53 was mixed with 2.5 picomoles (a ten fold excess) of various primers. The mixture was then heated to 65° C. and cooled slowly to room temperature to allow complementary primers to anneal to one another. His6D2 was then added, and the samples were incubated under conditions that permitted the covalent linkage reaction to proceed. To determine whether the preMS molecules were cleaved, the samples were run on a gel and the presence of the preMS and postMS molecules was detected using a phosphorimager.

This study revealed that an excess of unlabeled, single-stranded preMS2 molecules (oligo RRD1) effectively prevented the cleavage of the labeled, single-stranded preMS₁ (oligo 53), presumably by competing for a limited quantity of His6D2 molecules.

Double-stranded DNA was prepared by annealing complementary oligonucleotides (RRD7 and oligo 53 (the preMS molecule)). The RRD7:preMS, DNA duplex was resistant to cleavage by His6D2, consistent with the prediction that His6D2 cannot cleave double-stranded DNA without other auxiliary molecules (e.g., VirD 1). Thus, addition of nucleic acid molecules complementary to preMS molecules prevented preMS₁ cleavage, and DART synthesis.

His6D2 was also tested to determine whether it could perform the covalent linkage reaction on a preMS molecule bound to a nucleic acid complementary to the 3′ portion of preMS molecule, but not complementary to the 5′ portion of preMS molecule. Specifically, a portion of the preMS molecule that does not encompass the Recognition Sequence Motif was in a DNA duplex, and the Recognition Sequence Motif was single-stranded. His6D2 was found to cleave this species to form a DART.

When the reaction products were resolved on a gel, a molecule of the predicted size of a free postMS molecule was observed. This result can be explained if a fraction of the RRD3 melted off this portion of oligonucleotide 53 under the gel running conditions, which likely heated the samples above room temperature. Two oligonucleotides that were complementary to portions of the preMS molecule and exhibited melting temperatures close to the experimental temperature had no detectable effect on the covalent linkage reaction.

In summary, His6D2 was capable of performing the covalent linkage reaction on preMS molecules that were single-stranded around the Recognition Sequence Motif (RS). When the RS was encompassed within double-stranded DNA, His6D2 was unable to perform the covalent linkage reaction on this preMS molecule. This result confirmed that the double-stranded/single-stranded state of preMS at the RS can control whether a preMS molecule can participate in a covalent linkage reaction. Thus, the His6D2-mediated covalent linkage reaction performed in vitro was prevented by the addition of a molecule complementary to the Recognition Sequence Motif of the preMS molecule.

Temperature Dependence of His6D2 Activity

To assess the temperature sensitivity of His6D2 activity, the protein was exposed to different temperatures prior to performing the covalent linkage reaction. His6D2 was mixed with a 5′-radiolabeled preMS molecule on ice. Reactions were exposed to various temperatures for 20 minutes, returned to ice, and then assayed for covalent linkage activity.

His6D2 retained its activity after incubations at 0°, 37°, 42°, 45°, 60°, and 65° C., but its activity was eliminated after incubation at 70° C. or in the presence of EDTA. Further, when His6D2 was run through a G50 spin column, its activity was lost. This can be explained if His6D2 bound to the column. Thus, G50 column chromatography was not used to separate DARTs from preMS or postMS molecules. This study confirmed that His6D2 retained covalent linkage activity at a wide range of temperatures and that its activity was inhibited by the addition of EDTA.

Kinetics of The Covalent Linkage Reaction

A time course of DART synthesis was conducted. The reaction was started by contacting His6D2 and preMS molecules in the presence of magnesium. Aliquots of the reaction were withdrawn at different time points. This studyrevealed that equilibrium concentrations of postMS and DART molecules were observed at 30 minutes, but were not observed after five minutes. It further indicated that postMS molecules were not degraded by contaminants or other components in the reaction. The relative amounts of labeled postMS and preMS molecules did not change even when the reaction was incubated for 16 hours, indicating that the reaction had reached equilibrium. Thus, under the conditions studied, the covalent linkage reaction reached equilibrium between five and thirty minutes after its initiation.

Elimination of the Oligomerization Activity of VirD2 Full-Length VirD2 Forms Oligomers In Solution

Many DART applications require that DART LP components act as monomers in solution and do not self-associate. VirD2 epitope-tagged VirD2 fusions were used to test whether VirD2 and VirD2-derived DARTs oligomerize. A mixture of FLAG-tagged VirD2 DARTs and HA-tagged VirD2 DARTs was subjected to anti-FLAG antibody immunoprecipitation and Western blotting. The VirD2:HA DARTs co-immunoprecipitated with VirD2:FLAG DARTs, indicating that these proteins bound to one another. These data indicate that full-length VirD2 oligomerizes. Similar results were obtained when anti-HA antibodies were employed for the immunoprecipitation. These interactions were not fully disrupted by 250 mM NaCl or by the presence of the detergent triton X-100 (0.5%), suggesting that both VirD2 and VirD2-containing DARTs form oligomers in solution via specific binding interactions. The skilled artisan will appreciate that deletions, truncations, or mutations of VirD2 that do not form oligomers are especially useful as linkage polypeptides.

A Truncation of VirD2 Exhibits Linkage Activity and Does Not Oligomerize In Solution

Plasmids comprising the N-terminal 196 amino acids of VirD2 (designated VirD2-Nde) fused to the FLAG or VSV-G epitope tags were generated, designated D2-Nde:FLAG and D2-Nde:VSV-G. These plasmids were introduced into TOP10 cells. Protein expression was induced and the resulting proteins were purified and incubated with preMS oligonucleotides to synthesize DARTs as described. Western analyses revealed that D2-Nde:FLAG and D2-Nde:VSV-G exhibited linkage activity comparable to full-length VirD2. Thus, VirD2-Nde truncations of VirD2 retain the covalent linkage activity required to form DARTs.

Immunoprecipitation studies were performed to determine whether these VirD2-Nde truncations oligomerized. DARTs synthesized as described were incubated alone or together and the resulting solutions were subjected to immunoprecipitation with anti-VSV-G or anti-FLAG antibodies. Immunoprecipitated proteins and DARTs were subjected to gel electrophoresis and Western analysis. The experimental outline is indicated below:

Sample 1—VirD2-Nde:VSV-G immunoprecipitated with the anti-FLAG antibody; Sample 2—VirD2-Nde:FLAG immunoprecipitated with the anti-FLAG antibody; Sample 3—VirD2-Nde:VSV-G+VirD2-Nde:FLAG immunoprecipitated with the anti-FLAG antibody; Sample 4—VirD2-Nde:VSV-G immunoprecipitated with the anti-VSV-G antibody; Sample 5—VirD2-Nde:FLAG immunoprecipitated with the anti-VSV-G antibody; Sample 6—VirD2-Nde:VSV-G+VirD2-Nde:FLAG immunoprecipitated with the anti-VSV-G antibody.

Anti-FLAG immunoreactive bands corresponding to MP-LP and DARTs were detected in samples 2 and 3 only. Anti-VSV-G immunoreactive bands corresponding to MP-LP and DARTs were detected in samples 4 and 6 only. These data indicate that free VirD2-Nde:FLAG and VirD2-Nde:VSV-G proteins and DARTs derived from them can be immunoprecipitated and do not oligomerize. Thus, VirD2-Nde truncations are attractive Linkage Polypeptides because they retain covalent linkage activity and do not oligomerize, features useful for many DART applications.

### EXAMPLE

### DART Affinity Purification

Affinity purification of DARTs is typical for DarwinDART Directed Evolution, for preventing DART shuffling, and for removing undesirable DARTs, MP-LPs, proteins, nucleic acids, and the like. A DART comprising the HA epitope was affinity purified using a monoclonal anti-HA antibody.

Two epitope tagged DARTs comprising VirD2:HA:V5 and VirD2:V5 were synthesized as described above. DART-containing solutions were subjected to immunoprecipitation with the anti-HA antibody (12CA5) bound to a biotinylated secondary antibody bound to streptavidin beads. Following washes, samples were subjected to denaturing gel electrophoresis, and Western analysis using the anti-HA antibody.

Immunoreactive bands corresponding to free VirD2:HA:V5 protein and the VirD2:HA:V5 DART were detected in lanes derived from sample 1 (containing VirD2:HA:V5 and VirD2:HA:V5 DARTs) and sample 3 (containing VirD2:HA:V5, VirD2:HA:V5 DARTs, VirD2:V5 and VirD2:HA:V5 DARTs) but not sample 2 (containing VirD2:V5 and VirD2:V5 DART). These data demonstrate that a DART species (VirD2:HA:V5) was affinity purified from a mixture of proteins, nucleic acids, and other molecules. Further, the presence of a second DART did not interfere with the immunoprecipitation.

### EXAMPLE

### Detection of DARTs—Direct Detection of Radioactive DARTs

This study demonstrated that DARTs can be directly detected by radioactively labeling them. Three different preMS molecules (oligonucleotides 53, RRD1, and RRD1) were labeled at the 3′ end with ³²P-dCTP. DARTs were synthesized as described above by combining His6D2 and radioactively labeled preMS molecule under the appropriate reaction conditions. The reactions were incubated for one hour at 37° C. All samples were then subjected to denaturing gel electrophoresis followed by phosphorimager detection of radionuclides.

DARTs were detected as radioactively labeled bands of high molecular weight (estimated at 50-55 kD). Depending on the size of the preMS molecules, different sized DARTs were detected, corresponding to different predicted DART sizes. By contrast, when His6D2 was omitted from the reaction, no DARTs were detected. EDTA, which chelates divalent cations, also inhibited DART synthesis. An excess of cold oligonucleotide 53 competitor, which binds to RRD1, inhibited formation of radioactively labeled DARTs. DART formation was not inhibited by an excess of mutant oligos 58 or 59, which are not complementary to RRD1. The amount of DART formation (i.e., intensity of the band) was greatly reduced in a reaction containing RRD7, an oligo complementary to a preMS molecule that formed a double-stranded DNA complex with that molecule. The size of the DARTs increased when the reaction contained RRD3, an oligonucleotide that hybridized to the 3′ half of the preMS molecule and to the Molecular Shaft.

Several other observations were made in this study. First, the intensity of the high molecular weight DART band was the same for all samples, including DARTs with different Molecular Shafts. Because radiometric detection of DARTs did not depend on the ability of the Molecular Point, Linkage Polypeptide or Molecular Shaft to bind labeled detection molecules, radiometric detection is a method of direct DART detection. This study demonstrated that it is possible to directly detect DARTs by labeling their Molecular Shaft components. Moreover, it revealed that DART:Molecular Target (e.g., RRD3) complexes were stable and could be detected.

Direct Detection of Biotin-Labeled DARTs

Parallel studies to those described above were performed except that the preMS molecules were 3′-labeled with biotin-ddUTP instead of ³²P-dCTP. Covalent linkage reactions were performed for one hour at 37° C. split into three portions and run in different gels that were subjected to Western analysis with the anti-D2 antibody, the anti-His6 antibody, or SA-HRP.

Given starting conditions where [preMS)>[MP-LP], the predicted product and reactant concentrations at equilibrium were [MP-LP]=[DART]=[postMS]. His6D2 molecules that were inactive would not be incorporated into DARTs (i.e., shift to the DART mobility position on a gel). Thus, the maximum amount of His6D2 molecules expected to shift to the high molecular weight DART position=(Total His6D2 molecules−inactive His6D2 molecules)/2. It was observed that in the presence of an excess of preMS molecules, approximately half the His6D2 was free and half was in DARTs. This result confirmed that the majority of the His6D2 molecules were competent to react in the covalent linkage reaction.

Antibodies did not react equally with all DART species. Specifically, the anti-His6 and anti-His6D2 antibodies reacted poorly with the RRD1-derived DARTs but reacted well with the oligonucleotide 53-derived and oligonucleotide 63-derived DARTs. In contrast, the free His6D2 in these same reactions reacted equally well with both antibodies. The reduced Western signal for the RRD1-derived DART was believed to reflect reduced accessibility of the MP-LP pair in this molecule compared with the oligonucleotide 53-derived and oligonucleotide 63-derived DARTs. This conclusion was based in part on the findings that equal signals were obtained for oligonucleotide 53-derived, RRD1-derived, and oligonucleotide 63-derived DARTs in the radioactive DART study described above. The observed differences in DART : antibody binding indicated that for some applications, the Molecular Point, Linkage Polypeptide, and Molecular Shaft can be selected so that they will not interfere with the capacity of components in the DART (i.e. MP, LP, and MS) in which they reside to bind Molecular Targets.

The detection of DARTs with biotin-ddUTP at their 3′ termini demonstrated several features of DARTs. First, the DARTs have Molecular Shafts of a DNA-RNA fusion molecule (in this case the last nucleotide is a ribonucleotide). Second, the Molecular Point (His6 peptide) and MP-LP pair (His6D2) were able to bind non-DART Molecular Target molecules, antibodies in this case. Third, the Molecular Shafts of the DARTs were accessible for binding to Molecular Target molecules, in this case streptavidin-conjugated horse radish peroxidase (SA-HRP). Fourth, this accessibility was different for different DART species. Specifically, the RRD1-derived and oligonucleotide 63-derived DARTs reacted weakly with SA-HRP whereas the oligonucleotide 53-derived DART reacted strongly with SA-HRP. Differential binding was also observed between the oligonucleotide 53-, RRD1, and 63-derived DARTs and the anti-His6D2 and anti-His6 antibodies.

When His6D2 was co-incubated with a preMS molecule bound to a molecule complementary to the 3′ portion of preMS molecule, but not to the 5′ portion of preMS molecule (e.g., RRD3), a larger DART:RRD3 complex was observed. This directly demonstrated that the Molecular Shafts of DARTs were accessible for hybridization to a complementary nucleic acid and were simultaneously competent to bind SA-HRP. The anti-His6D2 antibody reacted with the DART:RRD3 complex whereas the anti-His6 antibody did not.

In conclusion, coincident detection methods confirmed the MP-LP-MS composition of DARTs and demonstrated that all three of these components were available to bind to Molecular Target molecules. Specifically, DARTs were detected using anti-His6 and anti-His6D2 antibodies as well as SA-HRP. In addition, a large fraction of the His6D2 molecules that were purified were competent to form DARTs. Moreover, DART:RRD3 complexes were detected and different DART species exhibited different MP, LP, and MS binding availability profiles. The observed differences in DART : Molecular Target binding indicated that for some applications, the Molecular Point, Linkage Polypeptide, and Molecular Shaft can be selected so that they will not interfere with the capacity of components in the DART (e.g., MP, LP, and MS) in which they reside to bind Molecular Targets.

### EXAMPLES

### Intermolecular DART Interactions—DART Hybridization to Nucleic Acids

Much of the information content of a DART lies in its Molecular Shaft. This information can be decoded by, for example, direct sequencing or by polymerase chain reaction amplification. Such methods can be useful in molecular evolution applications. The Molecular Shaft can also serve as a molecular address to target a DART to a nucleic acid complementary to the Molecular Shaft. This is illustrated by the DARTboard application.

For a variety of applications, the first step in determining the sequence of the Molecular Shaft of a DART is the contacting (e.g., hybridization) of the Molecular Shaft to a complementary nucleic acid molecule. This study demonstrated that the Molecular Shaft of a DART can hybridize to a complementary nucleic acid Molecular Target.

DARTs were labeled by reacting His6D2 with 3′-radiolabeled preMS molecule. DARTs were not observed when His6D2 was omitted from the reaction. In contrast, DARTs were observed when His6D2 and preMS molecules were co-incubated under suitable reaction conditions. DARTs were also observed when His6D2 was incubated with preMS molecules and a nucleic acid molecule, RRD3, complementary to the 3′ portion of the preMS molecule and to the Molecular Shaft. In this case, an upper molecular weight species corresponding to His6D2:RRD3 (MP-LP-MS:MT) was observed.

In a parallel study, DARTs were labeled by reacting His6D2 with preMS molecules in the presence of a 3′-radiolabeled nucleic acid molecule, RRD3, complementary to the 3′ portion of preMS and MS. DARTs were not formed when His6D2 was omitted from the reaction. In contrast, DARTs were observed when His6D2 and preMS (oligonucleotide 53) were co-incubated in the presence of labeled RRD3. In this case, an upper molecular weight species corresponding to His6D2:RRD3 (MP-LP-MS:MT) was observed. Furthermore, when His6D2 and a different preMS (RRD1), which was also complementary to RRD3, were co-incubated in the presence of labeled RRD3, a larger DART was produced. When His6D2 and a third preMS (RRD10), that was not complementary to RRD3, were co-incubated in the presence of labeled RRD3, no labeled DARTs were observed. This resulted demonstrated the sequence specificity of the DART : RRD3 hybridization events. This study demonstrated that the Molecular Shaft of a DART was available for hybridization to a complementary nucleic acid molecule (MT) and that this DART:MT complex was stable and could be detected.

This study demonstrated that the Molecular Shaft of a DART was available to hybridize to a nucleic acid molecule containing a complementary nucleic sequence. Previous studies did not distinguish between DART formation from a partially double-stranded preMS molecule and hybridization of the complementary nucleic acid sequence after DART formation. These studies were intended to directly test whether the Molecular Shaft of a DART was available for hybridization to complementary nucleic acid molecules.

His6D2 was allowed to react with biotinylated oligonucleotide 53 preMS molecule. EDTA was then added to stop His6D2 covalent linkage activity. Different oligonucleotides were then added to each reaction to assess the availability of the Molecular Shaft component of the resulting DARTs.

In one study, samples were run on a gel without heating them, transferred to membranes, and subjected to Western analysis with the anti-His6D2 antibody. In the control study, free His6D2 and the DART were detected, as expected. In the presence of oligonucleotide RRD3, and an excess of unlabeled oligonucleotide 53 preMS molecule (which is complementary to the Molecular Shaft), three different species (i.e., bands) were detected that corresponded to free His6D2, DARTs, and DART:RRD3 complexes. Because EDTA was added prior to the addition of the excess 53, His6D2 did not link itself to this population of preMS candidates.

In a parallel reaction containing RRD3 and an excess of unlabeled oligonucleotide 10 preMS molecules, which is not complementary to RRD3, two species were observed corresponding to the free His6D2 protein and the DART:RRD3 complex; no free DART was observed. Because His6D2 activity was inhibited in these reactions by EDTA, it was concluded that RRD3 hybridized to the Molecular Shaft of the intact DARTs and did not hybridize to preMS molecules prior to DART synthesis.

When an excess of unlabeled oligonucleotide 53 was added prior to the addition of RRD3, the DART: RRD3 complex was not observed. Thus, the RRD3: DART hybridization event was effectively prevented by the addition of an excess of unlabeled 53. In contrast, when an excess of oligonucleotide 10 was added prior to the addition of RRD3, the DART : RRD3 complex was observed. This result demonstrated the sequence specificity of the DART: RRD3 hybridization event.

In another study, a portion of each of the covalent linkage reactions described above were heated to 87° C., then run on a gel and transferred to membranes. The membranes were then subjected to Western blot analysis with the anti-His6D2 antibody. The heating and cooling first melted DNA duplexes and then allowed complementary nucleic acids to reanneal to one another. In the control, free His6D2 and DARTs were observed, as expected. In the presence of oligonucleotide RRD3 and an excess of unlabeled 53 preMS molecules, free His6D2 and a faint band corresponding to DARTs were observed; no DART: RRD3 complexes were observed. This result indicated that RRD3 hybridized to the excess 53 olignucleotide present and not to the Molecular Shafts of the DARTs.

In a parallel reaction containing RRD3 and an excess of unlabeled oligonucleotide 10 preMS molecules, which is not complementary to the Molecular Shafts, two species were observed that corresponded to free His6D2 and the DART : RRD3 complex; no free DARTs were observed. In this case, oligonucleotide 10 did not compete for hybridization to RRD3 and thus the DART : RRD3 complex was detected. As above, because His6D2 activity was inhibited in these reactions by EDTA and SDS loading dye present during the heating and cooling steps, it was concluded that RRD3 hybridized to the Molecular Shaft of the intact DARTs and did not hybridize to the preMS molecules prior to DART synthesis. When an excess of unlabeled oligo 53 was added prior to the addition of RRD3, the DART : RRD3 complex was not observed. Thus, the DART: RRD3 hybridization event was effectively stopped and prevented by the addition of an excess of unlabeled oligonucleotide 53. In contrast, when an excess of oligonucleotide 10 was added prior to the addition of oligonucleotide RRD3, the DART.: RRD3 complex was observed. This demonstrated the sequence specificity of the DART : RRD3 hybridization event.

These results demonstrated by several independent studies that the Molecular Shaft of a DART was capable of hybridizing to a complementary nucleic acid molecule in solution. Such binding (hybridization) can be used to decode and/or utilize the information in the Molecular Shafts of DARTs. Moreover, DART binding to nucleic acid Molecular Targets can be controlled by the order of addition of nucleic acid molecules.

DART Shuffling/MS Ligation

His6D2 was tested to determine whether it could catalyze ligation of MS₁ to postMS₂, a reaction involved in DART shuffling and DARTex. The covalent linkage reaction was performed in vitro with His6D2 and both labeled and unlabeled preMS₁ and preMS₂ molecules (oligonucleotides 53 and RRD1, respectively). Labeled preMS molecules, RRD1 and 53, alone did not show any ligation product (MS1-postMS2). Incubation of either labeled preMS molecules, RRD1 and 53, with His6D2 produced only the predicted small postMS products of the covalent cleavage reaction. When His6D2 was incubated in the presence of equimolar amounts of labeled RRD1 and oligo 53, a band corresponding to the ligation product of RRD1 MS (3′58 bp) with the 53 postMS molecule (5′ labeled 27 bp) for a net size of 85 bp was observed.

These data demonstrated that His6D2 could ligate the Molecular Shaft derived from oligo 53 to the postMS molecule derived from oligo RRD1. This reaction is involved in DARTex and DART shuffling. Because His6D2 activity can be inhibited by EDTA, the addition and removal of EDTA can be used to control the timing of the covalent linkage reaction associated with DART shuffling and/or DARTex.

Ligating DNA on the End of a DART a Prelude to DARTdance Ligation In Solution

Three reagents were prepared for this study: 1—DARTs were synthesized as described (supra) using VirD2:His6 and oligo RRD10; 2—The Matchmaker was prepared by mixing oligonucleotides 500 and 501 under conditions that allowed the complementary portions of these oligos to hybridize to one another. The resulting Matchmaker was partially double-stranded, with single-stranded termini capable of hybridizing to an Identity nucleic acid (which, as described below, is a nucleic acid molecule with a single stranded overhang referred to as a TAG, which TAG is complementary to the Matchmaker) and to the 3′ end of the Molecular Shaft of the DART; 3—The Identity nucleic acid consisted of a purified PCR product generated from the amplification of VirD2 gene sequences using the 206 and 210 primers. The PCR product was digested with PstI to provide a single-stranded terminus (TAG) capable of hybridizing to the Matchmaker. Different combinations of these reagents were mixed and then ligated with T4 DNA ligase. Finally, an aliquot of the ligation products was subjected to PCR analysis with the 207 and RRD10 primers. A 580 bp PCR product would result if the molecular shaft, Matchmaker, and Identity nucleic acid had all hybridized, ligated, and primed in the PCR reaction. 1—DART+Identity nucleic acid+Matchmaker; 2—DART+Identity nucleic acid+Matchmaker+ligase; 3—DART+Matchmaker; 4—DART+Matchmaker+ligase; 5—Identity nucleic acid +Matchmaker; 6—Identity nucleic acid+Matchmaker+ligase.

A PCR product of the expected size was observed in Sample 2 and not in any of the other controls. These data indicate that the Matchmaker hybridized to the Molecular Shaft of a DART and to the Identity nucleic acid molecule, and that these molecules were ligated to one another. The resulting DNA molecule was detected by PCR. These results demonstrate that DARTdance methods produce the expected results.

Ligation on a Solid Surface

In these studies, a DART was ligated to an Identity nucleic acid on a solid surface. Briefly, DARTs immobilized on agarose beads via an antibody were shown to become ligated to an Identity nucleic acid, also immobilized to a solid surface, via a Matchmaker as described below.

The following reagents were prepared for this study: 1—DARTs were synthesized in vitro as described using VirD2:His6 oligo RRD 10. 2—The Matchmaker was prepared by mixing oligonucleotides 500 and 501 under conditions that allowed the complementary portions of these oligos to hybridize to one another. The resulting Matchmaker was a partially double-stranded molecule with single-stranded termini capable of hybridizing to the Identity nucleic acid and to the 3′ end of the Molecular Shaft of the DART. 3—The Identity nucleic acid included a purified PCR product generated from 206 and 210 primers. The PCR product was biotinylated using biotin-ddUTP and terminal transferase and then it was digested with PstI and gel purified to provide a single-stranded terminus capable of hybridizing to the Matchmaker. 4—The anti-His6 antibody was biotinylated using the same methods for biotinylating DARTs (see above). A control Western indicated the biotinylated antibody could bind VirD2:His6 and bind to streptavidin HRP detection reagents. 5—Streptavidin agarose was incubated with 5 microliters of the biotinylated anti-His6 antibody and the biotinylated Identity nucleic acid DNA described above. Each reaction contained 250 picomoles of biotin binding capacity agarose and 50 picomoles of Identity nucleic acid PCR product.

DARTs and the agarose beads were then mixed at 4° Celsius for two hours, washed three times with 150 mM NaCl, 10 mM EDTA, and 20 mM TrisHCl pH 8.0 to remove non-bound DARTs, unreacted preMS molecules and other nucleic acid molecules. Beads were then washed in 1× ligase buffer. 50 picomoles of each Matchmaker oligo was then added to the beads. Finally, T4 DNA ligase and T4 ligase buffer were added to initiate the ligation reaction. Samples were then boiled, washed in the wash buffer indicated above, and boiled again. This should remove all material from the beads that is not bound via covalent interactions or a biotin-streptavidin interaction. Finally, PCR detection of the DARTdance progeny was performed with oligos 207 and RRD10.

Reactions containing DART, biotinylated anti-His6 antibody, beads, biotinylated Identity nucleic acid, and the Matchmaker yielded the expected PCR product whereas control reactions missing any of the various reaction components did not yield PCR products. These data constitute a simple proof-of-concept study for DARTdance. PCR analysis revealed that the expected DARTdance progeny were obtained only in reactions containing these components.

### EXAMPLE

### DARTboard Fabrication

Nucleic acid arrays are specific nucleic acid sequences immobilized on a support (e.g., glass side, silicon wafer, or nylon membrane) in an ordered fashion such that distinct species of nucleic molecules can be rapidly correlated with specific loci (e.g., spots) on that support. The utility of arrays emerges from the fact that tagged molecules (called ‘probes’) can be specifically targeted and attached (or ‘hybridized’), in a parallel fashion, to complementary nucleic acids immobilized on the support. In a complex mixture, each probe finds its complementary sequence pair and hybridizes to the spot where it resides. Detection of the probes assigns a signal to each nucleic acid species on the array, and ultimately provides information about the state (e.g., disease or otherwise) of the biological system from which the probes were extracted or from which they were generated. Arrays have proven to be extremely valuable tools for simultaneously measuring the expression levels of genes in cells, tissues, or organisms. In addition, they have been employed to sequence DNA, screen for the presence of viral or bacterial pathogens, or detect the presence of mutations associated with genetic diseases. DART technology provides method for generating protein arrays, or DARTboards. In a complex mixture, DART nucleic acid components can find and bind complementary nucleic acids present in a nucleic acid array, generating as a consequence, a self-assembling, and fully addressable protein array.

Materials and Methods

Materials

Oligonucleotides. The oligonucleotides used in these studies are described in Table 3.

Immunoreagents. Horse-radish peroxidase (HRP)-conjugated goat anti-mouse antibodies were obtained from Sigma Chemical Co. Anti-6×HIS monoclonal antibodies were obtained from Sigma Chemical Co.

Streptavidin-biotin reagents. HRP-Streptavidin and NHS-SS-biotin were obtained from Pierce Chemical Co, and employed as per the manufacturer's recommendations.

Methods

Biotinylating DARTs

DARTs were labeled with biotin as follows: First, DARTs were synthesized. Fractions enriched in affinity purified His-virD2 (3 μg) proteins were incubated with various oligonucleotides (400 pM) harboring T-border sequences for 60 min at 37° C. (30 μl reaction volume) in the presence of MgCl₂ (5 mM)-containing reaction buffer. Next, the DART-containing mixtures were dialyzed (10 kDa MWCO dialysis tubing, Sigma Chemical Co) overnight against one liter of dialysis buffer (50 mM carbonate buffer, pH 7.0) at 4° C. in an eight-well microdialyzer apparatus (Pierce Chemical Co.). The dialyzed material was recovered, and then incubated with one-tenth volume (i.e., 3 μl) of NHS-SS-biotin (Pierce Chemical Co.) (10 mg/ml in H₂O) for 2 hours on ice in the dark. During this incubation, biotin became covalently coupled to DART molecules. Next, the biotinylation reaction was stopped by adding 3 μl of EDTA (500 mM, pH 8.0) to the reaction mixture. The biotinylated material was dialyzed as before against one liter of 150 mM NaCl, 10 mM EDTA, 50 mM Tris buffer, pH 7.0, to remove unincorporated NHS-SS-biotin. Finally, the dialyzed material was recovered (30 μl) from the dialysis apparatus, and then employed in the various studiess described herein.

DNA Array FABRICATION

Simple DNA arrays were fabricated for DARTboard studies. To fabricate these arrays, a uniform grid was first drawn onto nylon supported pure nitrocellulose (Osmonics, Inc.) with a pencil. Assorted oligonucleotides (200 pM) were then spotted at regular intervals on this grid. Next, the oligonucleotide-containing membrane was air-dried and UV irradiated for 2 min to immobilize the nucleic acids. The membrane harboring the arrayed oligonucleotides was finally immersed briefly in ddH₂O before being placed in blocking buffer (3% bovine serum albumin (BSA), 150 mM NaCl, 10 mM EDTA, 50 mM Tris, pH 7.0) for 1 hour at room temperature before use. In some studies, 10 μg/ml salmon sperm DNA was also included in the blocking buffer.

DARTboard Fabrication

To fabricate DARTboards, DARTs were first synthesized. Next, DART probes (30 μl) were diluted in blocking buffer (5 ml) and incubated (at room temperature) in the presence of a pre-fabricated and pre-blocked DNA array. During this incubation (1 hour), the nucleic acid components of DART molecules (i.e., the Molecular Shafts) hybridized with their complementary nucleic acids immobilized on the membrane. The incubation solution was removed, and the membrane washed three times (10 min/wash) with blocking buffer. DARTs immobilized on membranes were then detected as described below.

Detecting DARTs on DARTboards

To detect DARTs on DARTboards, immunocytochemical or biotin-streptavidin chemistry methods were employed.

Immunocytochemical Detection of HIS-VirD2

DARTs hybridized to complementary nucleic acids immobilized on supported nitrocellulose membranes were incubated in the presence of primary monoclonal antibodies (1:15,000 dilution in blocking buffer) raised against 6×HIS epitopes. During this incubation, antibodies recognized and bound to the 6×HIS non-nucleic acid components of immobilized DART molecules. The membranes were washed three times (10 min/wash) in the presence of antibody-free blocking buffer to remove unbound primary antibodies, and then incubated in the presence of HRP-conjugated goat anti-mouse secondary antibodies (1: 15,000 dilution in blocking buffer) for I hour. The membranes were washed three more times in antibody-free blocking buffer as described above, and then rinsed briefly in rinse buffer (150 mM NaCl, 10 mM EDTA, 50 mM Tris, pH 7.0). Finally, the immobilized DARTs were detected using ECL chemiluminescent detection methods as per the manufacturer's recommendations (Pierce, Inc). All incubation steps were carried out at room temperature with gentle agitation.

Biotin-Streptavidin Detection of Biotinylated DARTs

Biotinylated DARTs hybridized to complementary nucleic acids immobilized on supported nitrocellulose membranes were incubated (1 hour) in the presence of HRP-conjugated streptavidin (1:15,000 in blocking buffer). During this incubation, non-covalent streptavidin-biotin complexes were formed. The membranes were washed three times (10 min/wash) in the presence of streptavidin-free blocking buffer, and the immobilized DARTs detected using ECL chemiluminescent detection chemistry as per the manufacturers instructions (Pierce, Inc). All incubation steps were carried out at room temperature with gentle agitation.

Results

To illustrate the ways in which DARTs can be employed to fabricate novel protein arrays, a variety of studies were performed.

The ability of DART Molecular Shafts (MS) to hybridize to complementary nucleic acid sequences immobilized on a substrate and to direct the corresponding DART Molecular Point (MP) to those same locations was examined. Affinity purified HIS-virD2 protein and oligonucleotides PD53, PD60, and RRD1, were used to synthesize three DARTs in vitro, HIS-virD2-PD53, HIS-virD2-PD60, and HIS-virD2-RRD1 (henceforth denoted as 53-DART, 60-DART, and RRD1-DART). Next, a simple DNA array was fabricated using oligonucleotides both complementary (RRD3 and RRD7) and non-complementary (PD62) to the PD53 and RRD1 Molecular Shaft sequences. This array also harbored specific loci at which fully-intact PD53 oligonucleotide sequences were located. These PD53 oligonucleotides contained A. tumefaciens virD2 cleavage and joining recognition sequences (the Recognition Sequence Motif). All three DART species, or HIS-virD2 proteins alone, were incubated in the presence of the DNA array to examine the ability of DARTs (and HIS-virD2 proteins) to target to appropriate immobilized nucleic acids.

When HIS-virD2 protein alone was used to probe the DNA array, a strong signal was observed at the locus harboring the fully-intact PD53 oligonucleotide, demonstrating that HIS-virD2 protein possesses the capacity to couple itself to immobilized oligonucleotides containing the appropriate Recognition Sequence Motif. When the DNA array was incubated in the presence of 53-DARTs, the 6×HIS-tag Molecular Points of these DART molecules were found, by immunochemical methods using monoclonal antibodies directed against 53-DART HIS-tag epitopes, to be localized at spots on the array harboring oligonucleotide sequences complementary to the Molecular Shaft of 53-DARTs. As expected, similar results were obtained when RRD1-DARTs were employed to probe the array. Importantly, no signal was detected at spots on the array harboring non-complementary nucleic acids or when PD53 oligonucleotides alone or PD60-DARTs were employed to probe the array. These observations confirmed that the Molecular Shaft of DARTs can specifically direct the DARTs to corresponding nucleic acids on DNA arrays.

A series of studies was also conducted in which DART Molecular Shafts were masked by pre-incubating them with complementary oligonucleotides. Masking the Molecular Shafts of the DARTs (by hybridizing them to complementary nucleic acids) should prevent DART targeting. When 53-DARTs were first incubated in the presence of a large excess of complementary PD57 oligonucleotides (but not non-complementary control PD62 oligonucleotides), 53-DART:PD57 molecular complexes (that is, molecular complexes harboring PD53:PD57 heteroduplexed DNA) were formed. When this complex was incubated in the presence of the DNA array, no 53-DARTs were found localized to loci (e.g., spots ) harboring the complementary nucleic acid sequences. Importantly, when control probes derived from reaction mixtures harboring 53-DART and PD62 molecules were employed, 53-DARTs were found specifically localized to the expected loci. These data confirmed that DART Molecular Shaft components can target DART Molecular Points to their appropriate locations on the array.

The ability of DARTs in a mixture of DARTs to bind to the corresponding nucleic acids on a nucleic acid array in the presence of other competing DARTs and nucleic acids was also confirmed. In one study, large amounts (10 μg/ml) of salmon sperm DNA was included in the blocking and incubation buffers. Under these conditions, DART targeting to appropriate loci on the array was observed. Next, the ability of DARTs to bind to the corresponding nucleic acid on the array in the presence of competing DARTs was confirmed. Two DART species were synthesized. The first DART species (53-DART) harbored DNA sequences complementary to those present in the DNA array (RRD3 and RRD7). The second DART species (60-DART) lacked these complementary sequences. When a reaction mixture containing 53-DART and 10-fold or 100-fold excess of 60-DART was contacted with the DNA array, 53-DART targeting to appropriate locations on the DNA array was not inhibited. Although these data demonstrated that no change in the amount of Molecular Point signal was observed in the presence of the competing DART, the data do not provide insight into the origins of the observed signal. The observed signal could arise either from the appropriate targeting of 53-DARTs, or from the mis-targeting of non-specific 60-DARTs to spots on the array harboring oligonucleotides complementary to PD53. To distinguish between these possibilities, a model system employing DARTs harboring two distinct MP components was developed and employed.

To examine whether two different DARTs, after being mixed together in a single reaction tube, could sort independently on a DNA array, two DARTs (53-DARTs and Biotin-60-DARTs) were synthesized that could be distinguished from one another using separate DART detection systems. Monoclonal antibodies directed against the 6×HIS-tag recognized (by Western analysis) appropriate epitopes present in HIS-virD2-PD53 DARTs (53-DARTs). These same antibodies did not cross-react with biotinylated DARTs (biotin-HIS-virD2-PD60, or biotin-60-DARTs). Similarly, HRP-conjugated streptavidin (HRP-streptavidin) recognized biotin conjugates in biotin-60-DARTs, but failed to recognize non-biotinylated 53-DARTs (data not shown). This lack of cross-reactivity provided a convenient system for studying DART sorting on DNA arrays.

When the biotin-60-DARTs and 53-DARTs were first mixed together and then incubated in the presence of a DNA array harboring sequences complementary to those present in these DARTs, the DARTs sorted (i.e., bound) to their expected locations on the array. No detectable mis-sorting was observed: 53-DARTs were found localized to spots harboring complementary RRD7 or RRD3 sequences, and biotin-60-DARTs were found localized to spots harboring complementary PD72 sequences. No 53-DART signal was found localized to spots harboring PD72 or other nucleic sequences. The observations confirmed that DART sorting on the nucleic acid array was specifically driven by the identities of DART Molecular Shaft sequences.

In another study, one DART species (53-DARTs) was contacted with oligonucleotides (PD57) harboring sequences complementary to those contained in the 53-DART Molecular Shaft. This ‘masked’ DART was added to a reaction mixture containing ‘unmasked’ biotin-60-DARTs. When this reaction mixture was incubated in the presence of the DNA array, the unmasked molecule appropriately sorted to its expected location, but the masked counterpart failed to sort appropriately, and was therefore not detected. These data confirmed that DART sorting requires unmasked and therefore hybridization-accessible DART MS DNA components.

DARTboard Variation: Generating DARTboards By Linking MP-LPs to PreMS Capture Reagents on a DNA Array

DART MP-LP protein components were contacted with a DNA array comprising preMS capture reagents to generate a DARTboard. Specifically, VirD2:His6 was incubated with a DNA array comprising preMS oligonucleotides on a nitrocellulose membrane. Twenty-four spots contained the PD53 preMS oligonucleotide and twenty four spots contained oligonucleotide PD62. PD53 includes VirD2 recognition sequences whereas PD62 does not. In another study, a DNA array containing spots harboring oligonucleotide RRD10 or PD62 was used. RRD10 contains VirD2 recognition sequences, and is non-identical to PD53 outside the VirD2 recognition sequence region. The resulting DARTs were detected immunochemically using monoclonal antibodies directed against HIS6 epitopes. In both studies, anti-His6 immunoreactivity was detected at array spots harboring PD53 or RRD10. No immunoreactivity was detected at spots harboring PD62. Thus, DARTboards were generated by linking MP-LPs to preMS capture reagents in a DNA array.

### EXAMPLE

### Synthesizing a Plurality of DARTs

Plasmid constructs with genes encoding VirD2 fused to the VSV-G epitope, FLAG epitope, HA epitope, V5 epitope, His6 epitope, or MYC epitope under control of the arabinose-inducible AraC promoter were introduced into TOP10 cells (Ogden et al., 1980, Proc Natl. Acad. Sci 77:3346-50, Schleif, 1992, Ann. Rev. Biochem. 61:199). Expression was induced according to established methods (See Invitrogen pBAD TOPO TA Expression Kit manual version J 2001 and references therein) and the fusion proteins were purified using Nickel-agarose affinity chromatography (See Qiagen QlAexpressionist handbook Third edition 1997 and references therein). These proteins were covalently coupled to preMS-containing oligonucleotides in vitro (as described supra) and detected by Western analysis using monoclonal antibodies directed against molecular point elements as described. By this means a plurality of DARTs was generated, each comprising a different molecular point (e.g., HA, VSV-G, MYC, V5, or His6 epitopes) and a distinct Molecular Shaft (derived from oligonucleotides, such as RRD10 and PD53 comprising VirD2 recognition sequences). These activity assays revealed that the epitope tags did not interfere with the efficiency of the in vitro covalent linkage activity of these different MP-LP pairs.

1. A DART comprising a Molecular Shaft covalently linked to a Linkage Polypeptide covalently linked to a Molecular Point.

2. The DART of claim 1, wherein the Linkage Polypeptide is an autocatalytic Linkage Polypeptide.

3-7. (Canceled)

8. The DART of claim 2, wherein the Linkage Polypeptide is a replication protein of a single-stranded DNA mycobacterial virus or bacteriophage or a nicking or relaxase enzyme of a bacterial plasmid.

9. The DART of claim 8, wherein the plasmid is a narrow host range plasmid or a broad host range plasmid.

10. The DART of claim 9, wherein the narrow host range plasmid is F, R1, R100, 61B4-K98, P307, or pED208.

11. The DART of claim 10, wherein the nicking or relaxase enzyme is TraI or a derivative of TraI lacking the ability to catalyze the formation of a covalent bond with a double stranded nucleic acid.

12. The DART of claim 9, wherein the broad host range plasmid is RP4, RP1, RK2, R18, R68, or R751.

13. The DART of claim 8, wherein the plasmid is a mobilizable plasmid.

14. The DART of claim 13, wherein the mobilizable plasmid is IncQ, RSF1010, R300B, or R1162.

15. The DART of claim 14, wherein the nicking or relaxase enzyme is MobA.

16. The DART of claim 8, wherein the plasmid is an R388-related plasmid.

17. The DART of claim 16, wherein the nicking or relaxase enzyme is TrwC.

18. The DART of claim 8, wherein the nicking or relaxase enzyme is a VirD2 enzyme of an A. tumefaciens Ti plasmid.

19-28. (Canceled)

29. The DART of claim 2, wherein the Linkage Polypeptide selectively catalyzes the formation of a covalent bond between itself and a single stranded nucleic acid or a double stranded nucleic acid.

30. The DART of claim 2, wherein the Linkage Polypeptide is covalently attached to the 5′-end or the 3′-end of a nucleic acid.

31. The DART of claim 2, wherein the Linkage Polypeptide is a fusion protein.

32. The DART of claim 31, wherein the fusion protein comprises a protein that catalyzes the formation of a covalent link between itself and a nucleic acid, and an accessory protein.

33. The DART of claim 9 32, wherein the protein that catalyzes the formation of a covalent link between itself and a nucleic acid is VirD2 and the accessory protein is VirD1.

34-50. (Canceled)

51. The DART of claim 1, wherein the Molecular Shaft encodes a fusion polypeptide comprising the Linkage Polypeptide and the Molecular Point.

52. (Canceled)

53. The DART of claim 1, wherein the Molecular Point comprises a polypeptide or an antibody.

54. The DART of claim 1, wherein the biological function of the Molecular Point is unknown.

55. The DART of claim 1, wherein the Molecular Point is a mutant or variant of a known protein.

56-75. (Canceled)

76. A method of making a DART, comprising contacting a preMS with Linkage Polypeptide covalently linked to a Molecular Point under conditions that allow the formation of a DART, wherein the Linkage Peptide is an autocatalytic Linkage Polypeptide, is a substrate for a transcatalytic linking protein, or is a substrate for a trans-complementary linking protein.

77. A DART library comprising a plurality of DART species, the DARTs comprising a Molecular Shaft covalently linked to a Linkage Polypeptide covalently linked to a Molecular Point.

78. The DART library of claim 77, wherein the DART species in the library comprise different Molecular Points or different Molecular Shafts.

79. The DART library of claim 77, wherein the Molecular Shafts of the DART species encode a fusion polypeptide comprising the Linkage Polypeptide and Molecular Point of the DART species.

80-84. (Canceled)

85. The DART library of claim 77, which is an in vitro library.

86-135. (Canceled)

136. A Modular DART Assembly comprising a first DART complexed with a second DART, wherein (i) the first DART comprises a first Molecular Point covalently linked to a first Linkage Polypeptide covalently linked to a first Molecular Shaft; (ii) the second DART comprises a second Molecular Point covalently linked to a second Linkage Polypeptide covalently linked to a second Molecular Shaft; and the first DART and the second DART are complexed through an interaction between the first Molecular Point and the second Molecular Point, through an interaction between the first Molecular Shaft and the second Molecular Shaft or through an interaction between the first Molecular Point and the second Molecular Shaft.

137. A composition comprising: (a) a first DART, the first DART comprising a first Molecular Shaft covalently linked to a first Linkage Polypeptide covalently linked to a first Molecular Point; and (b) a Molecular Target bound to the first DART.

138-142. (Canceled)

143. The composition of claim 137, wherein the Molecular Target is not a DART.

144. The composition of claim 137, wherein the Molecular Target is covalently or non-covalently attached to the first Molecular Shaft, the first Molecular Point, or the first Linkage Polypeptide.

145. The composition of claim 143, wherein the Molecular Target is a small molecule or a drug.

146-181. (Canceled)


SEQUENCE LISTING (46 sequences)

SEQ ID NO: 1 - protein, 14 aa, Escherichia coli
FAELVKYITDEQGK

SEQ ID NO: 2 - protein, 14 aa, Escherichia coli
  VARIANT (7): Any amino acid
TYHLLVXSFRAGEK

SEQ ID NO: 3 - protein, 22 aa, Escherichia coli
QRSVSVHHDTDNLHIHIAINKI

SEQ ID NO: 4 - protein, 14 aa, Escherichia coli
FAGLANYITDAQSK

SEQ ID NO: 5 - protein, 14 aa, Escherichia coli
  VARIANT (7): Any amino acid
TYHLIVXSFRAGEQ

SEQ ID NO: 6 - protein, 22 aa, Escherichia coli
QRISAVHNDTDNLHIHIALNKI

SEQ ID NO: 7 - protein, 14 aa, Escherichia coli
VSRLTGYIREPERE

SEQ ID NO: 8 - protein, 14 aa, Escherichia coli
  VARIANT (7): Any amino acid
INHYVLXSWREGEQ

SEQ ID NO: 9 - protein, 22 aa, Escherichia coli
QAIYGLHADTDNLHLHLAINRV

SEQ ID NO: 10 - protein, 14 aa, Escherichia coli
FSRLVDYATRLRNE

SEQ ID NO: 11 - protein, 14 aa, Escherichia coli
  VARIANT (7): Any amino acid
VFHYIVXSWQSHES

SEQ ID NO: 12 - protein, 22 aa, Escherichia coli
QYVSAVHTDTDNLHVHVAVNRV

SEQ ID NO: 13 - protein, 14 aa, Agrobacterium tumefaciens
IINQLEYLSRKGRL

SEQ ID NO: 14 - protein, 14 aa, Agrobacterium tumefaciens
  VARIANT (7): Any amino acid
TTHIIVXSFPAGTS

SEQ ID NO: 15 - protein, 22 aa, Agrobacterium tumefaciens
NYLTAFHIDRDHPHLHVVVNRR

SEQ ID NO: 16 - protein, 22 aa, Agrobacterium tumefaciens
NYLRAGHIDRDHPHLHVVVNRR

SEQ ID NO: 17 - protein, 14 aa, Agrobacterium tumefaciens
  VARIANT (7): Any amino acid
TTHIIVXSFPAGTD

SEQ ID NO: 18 - protein, 22 aa, Agrobacterium tumefaciens
NYLRAYHVDRDHPHLHVVVNRR

SEQ ID NO: 19 - protein, 14 aa, Escherichia coli
ASRAINYAEERAEE

SEQ ID NO: 20 - protein, 14 aa, Escherichia coli
  VARIANT (14): Any amino acid
QAHTVIQSFKPGEX

SEQ ID NO: 21 - protein, 22 aa, Escherichia coli
QVAVYTHTDKDHYHNHIIINSV

SEQ ID NO: 22 - protein, 22 aa, Escherichia coli
QVAVYTHTDKDHYHNHIVINSV

SEQ ID NO: 23 - protein, 14 aa, Escherichia coli
TSRAINYAEERAEE

SEQ ID NO: 24 - protein, 14 aa, Escherichia coli
  VARIANT (14): Any amino acid
EGHVVIQSFKPGEX

SEQ ID NO: 25 - protein, 22 aa, Escherichia coli
QVAVYTHNDTDHVHNHIVINSI

SEQ ID NO: 26 - protein, 7 aa, Simian virus 40
PKKKRKV

SEQ ID NO: 27 - DNA, 10 bp, Agrobacterium tumefaciens
tatatcctgt

SEQ ID NO: 28 - DNA, 10 bp, Escherichia coli
cctatcctgc

SEQ ID NO: 29 - DNA, 9 bp, Poliovirus
uuaaaacag

SEQ ID NO: 30 - DNA, 14 bp, Adenovirus
catcatcataatat

SEQ ID NO: 31 - DNA, 9 bp, Artificial Sequence
  Description of Artificial Sequence Recognition Sequence Motif
tatatcctg

SEQ ID NO: 32 - protein, 15 aa, Artificial Sequence
  Description of Artificial Sequence Linker peptide
GGGGSGGGGSGGGGS

SEQ ID NO: 33 - protein, 36 aa, Artificial Sequence
  Description of Artificial Sequence His6 tag
MGSSHHHHHHSSGLVPRGSHMASMTGGQQMGRGSEF

SEQ ID NO: 34 - DNA, 60 bp, Artificial Sequence
  Description of Artificial Sequence Primers
gctagctttacccgccaatatatcctgtcaaacacaatgcccgatcgcgctcaagtaatc

SEQ ID NO: 35 - DNA, 41 bp, Artificial Sequence
  Description of Artificial Sequence Primers
gctagcttcacctatcctgcccggtgattgccaagcacgtc

SEQ ID NO: 36 - DNA, 41 bp, Artificial Sequence
  Description of Artificial Sequence Primers
gctagcccttagctcctcctagcggttatcctaacgagctc

SEQ ID NO: 37 - DNA, 70 bp, Artificial Sequence
  Description of Artificial Sequence Primers
caatatatcctgtcaaacacaatgcccgatcgcgctcaagtaatcattcgcattgtgccaggaggtggaa

SEQ ID NO: 38 - DNA, 49 bp, Artificial Sequence
  Description of Artificial Sequence Primers
ttccacctcctggcacaatgcgaatgattacttgagcgcgatcgggcat

SEQ ID NO: 39 - DNA, 43 bp, Artificial Sequence
  Description of Artificial Sequence Primers
gattacttgagcgcgagatatattggcgggtaaagctagcaaa

SEQ ID NO: 40 - DNA, 60 bp, Artificial Sequence
  Description of Artificial Sequence Primers
gctagctttacccgccaatatatcctgtcaatgttgagtttcagcgttgttaaatcggca

SEQ ID NO: 41 - DNA, 36 bp, Artificial Sequence
  Description of Artificial Sequence Primers
gaattcctcgagggtccccccgcgcccatcgtcgcg

SEQ ID NO: 42 - DNA, 36 bp, Artificial Sequence
  Description of Artificial Sequence Primers
gaattcctcgagagcatatgtgattggtcgctctgc

SEQ ID NO: 43 - DNA, 41 bp, Artificial Sequence
  Description of Artificial Sequence Primers
ggcagtgccggaaactattacaccgacaaggataatctgca

SEQ ID NO: 44 - DNA, 67 bp, Artificial Sequence
  Description of Artificial Sequence Primers
gattatccttgtcggtgtaatagtttccggcactgcctgccgatttaacaacgctgaaactcaacat

SEQ ID NO: 45 - DNA, 25 bp, Artificial Sequence
  Description of Artificial Sequence Primers
gggaaacgacaatctgagctccacc

SEQ ID NO: 46 - DNA, 25 bp, Artificial Sequence
  Description of Artificial Sequence Primers
gacgcttagacaacttaataacaca